(19) United States
(12) Patent Application Publication
(10) Pub. No.: US 2011/0172107 A1
(43) Pub. Date: Jul. 14, 2011

(54) ASSAY FOR IDENTIFYING AGENTS THAT MODULATE EPIGENETIC SILENCING, AND AGENTS IDENTIFIED THEREBY

(75) Inventors: Richard A. Katz, Elkins Park, PA (US); Anna Marie Skalka, Princeton, NJ (US)

(73) Assignee: FOX CHASE CANCER CENTER, Jenkintown, PA (US)

(21) Appl. No.: 12/736,702

(22) PCT Filed: Apr. 30, 2009
(86) PCT No.: PCT/US09/02677

§ 371 Date: Feb. 22, 2011

Related U.S. Application Data
(60) Provisional application No. 61/125,932, filed on Apr. 30, 2008.

(51) Int. Cl. C12Q 1/68; C40B 30/00; C12N 5/071; C12N 9/78; C07K 14/47; C12N 9/10; C12N 9/00; C12N 9/02

(52) U.S. Cl. 506/7; 435/6.1; 435/366; 435/227; 530/350; 435/193; 435/183; 435/189

(57) ABSTRACT

A high throughput RNAi-based assay for identify factors involved in maintaining epigenetic silencing is disclosed. The assay measures reactivation of a silent reporter gene in cells, resulting from RNAi-based knockdown in target mRNA. RNAi-based screening of these silent reporter cells has identified known enzymes that place or remove epigenetic marks on histones, as well as non-enzymatic proteins that function in silencing or in transfer of marks during S-phase. In addition, the screen has been used to identify a number of novel gene products involved in epigenetic silencing, which are also disclosed.

### REFERENCE TO SEQUENCE LISTING

The Sequence Listing submitted on compact disk is hereby incorporated by reference. The file on the disk is named 046598-0005-WO-00.txt. The file is 6,591 kb and the date of creation is Apr. 30, 2009.

### BACKGROUND OF THE INVENTION

Each cell type in a multi-cellular organism may express only a characteristic subset of genes, yet largely retain the complete DNA blueprint. This programmed use of the genetic code is mediated by events that are defined as epigenetic: heritable changes in gene expression without changes in the nucleotide sequence. Epigenetic programming participates in the shaping of cellular identity by mediating heritable shutoff, or expression, of specific gene sets. Epigenetic processes thereby account for much of the selectivity and plasticity with respect to execution of gene expression. Epigenetic controls play a role in a variety of biological phenomena including cell differentiation, gene imprinting, X-chromosome inactivation and silencing of foreign DNA.

Rather than serving simply an organizational role for DNA packing, the histone components of nucleosomes play important roles in epigenetic control of gene expression. Epigenetic regulators have now been implicated in a variety of normal and disease processes including stem cell identity and cancer, respectively. While the molecular nature of genetic inheritance has been appreciated for the last half century, it has become obvious that epigenetic inheritance is more difficult to describe with simple paradigms.

Several basic features of epigenetic control are firmly established. Epigenetic regulation can be mediate by placement or removal of small chemical marks on chromatin (DNA and histones) and such modifications provide the “heritable” instructions for gene expression or gene silencing. Some of these marks had been well studied for decades, but their associations with gene function were simply correlative. Several breakthrough findings over the past decade launched the field as it is known it today. The first finding was that a histone modifying enzyme could control gene expression. It had been well established that transcribed genes were enriched for acetylated histones, but the identification of a histone acetyltransferase (HAT) as a transcriptional activator was the key finding that stimulated the current excitement surrounding epigenetic controls. It is now appreciated that histone tail acetylation provides binding sites for positive acting factors and that histone deacetylases (HDACs) remove acetyl groups and thereby antagonize the activity of HATs. These modifications provided a paradigm, leading to the “histone code” hypothesis, whereby a translatable set of histone modifications can mediate epigenetic processes. Similarly, DNA methylation had been associated with transcriptional repression, yet a causal relationship had not been established. The identification of methyl binding domain proteins (MBD) suggested one means by which repressive complexes could be recruited to methylated DNA. Evidence for a causal role for DNA methylation in epigenetic silencing is now generally accepted, but is still open to experimental investigation.

The current view of the histone code hypothesis is that enzymes can place or remove numerous epigenetic marks on histones, primarily on the N-terminal tails, and that these modifications are read in a specific manner to control gene expression. The numerous enzymes that place marks (e.g. acetylation or methylation on lysines residues) are described as “writers” of the histone code. The activities of these enzymes are antagonized by “erasers,” enzymes that remove these marks. “Readers” and “erasers” recognize the epigenetic marks and implement gene activation or repression. The readers typically contain modular domains that recognize specific histone modifications. Similarly, DNA methylation marks are recognized by proteins containing modular methyl binding domains (MBDs). Lastly, “chromatin remodeling complexes” also play critical roles in epigenetic control, as they can mediate accessibility to chromatin of both positive and negative epigenetic regulators and thus are viewed as participants in epigenetic regulation.

A key aspect of epigenetic regulation in development is the temporal and positional placement of epigenetic marks on histones and DNA. That is, the enzymes responsible for epigenetic marking activities must be targeted to specific genes at the appropriate time and be sustained through cell division. As such, the epigenetic marking activities can be generally classified as: initiation (de novo placement of marks) or maintenance during chromatin replication and cell division.

There is also a coordination between placement of epigenetic marks on DNA and histones. For example, DNA methylation and histone H3 lysine 9 (H3K9) methylation are generally regarded as marks associated with repressive heterochromatin. A seminal finding was that histone methylation can direct DNA methylation, a facet of what has been described as a “cooperative and self-reinforcing organization of the chromatin and DNA modifying machinery.”

As epigenetic instructions are by definition heritable, there is significant interest in the mechanisms by which histone code modifications and DNA methylation patterns are “inherited” through S-phase and mitosis. In the case of DNA methylation, it is understood that the hemi-methylated DNA produced during DNA replication provides a substrate for continued placement of methylation marks. In the case of histone marks, it is unclear as to whether this is accomplished by mechanisms whereby existing marks guide the placement of new marks during S-phase, or if the continued presence (or rebinding) of factors is required. It is likely that both enzymatic markings, as well as factor positioning, mediate “inheritance” of epigenetic marks during S-phase and mitosis. It has been noted that there is no direct proof for transfer of histone modifications to new chromatin during S-phase and an alternative view is that at least some types of epigenetic “inheritance” can be explained by classic models for transcriptional repressor binding.

Although many of the above concepts are well-supported, it has become apparent that the biochemical marks on chromatin may signify more complex and dynamic processes then had been previously thought. For example, H3K9 methylation was believed to be an exclusive mark for repressive heterochromatin, providing a recognition mark for heterochromatin protein 1 (HP1). More recent studies have indicated that this mark is also found in the body of active genes, perhaps signifying “transcriptional memory” or a mechanism to prevent inappropriate transcriptional initiation in the body of an active gene. It has also become appreciated that certain types of heterochromatin are transcribed, leading to RNAi-directed chromatin silencing of these transcribed regions. This counterintuitive mechanism highlights the potential complexities of epigenetic silencing systems. Also, numerous studies have indicated that histone modifications can be highly context-dependent and thus are not readily translatable as a simple code. Such intricacies can be explained by multivalent interactions on single histone tails. So-called “bivalent genes” have revealed further complexities. These genes contain both activating and repressive histone modifications; such relationships may signify genes that are poised for activation.

Thus, there is a need in the art to discover and characterize new epigenetic markers and regulators in cells, preferably through the use of a robust model system that is reproducible and amenable to use in a high throughput manner. The present invention satisfies that need.

### SUMMARY OF THE INVENTION

One aspect of the invention features a method of identifying gene products that are involved in epigenetic silencing, comprising: (1) providing a cell line comprising a genome into which is integrated an epigenetically silent reporter gene; (2) providing a mRNA inhibitor capable of inhibiting expression of a target mRNA, the product of which is suspected of being involved in the epigenetic silencing; (3) introducing the mRNA inhibitor into the cell line, thereby inhibiting expression of the target RNA; and (4) detecting an increase in expression of the reporter gene, the increase in expression being indicative that the product of expression of the target mRNA is involved in the epigenetic silencing.

The cell line can be of human origin or it can originate from another species. In one embodiment, the cell line is a HeLa cell line. In one embodiment, the silent reporter gene encodes a green fluorescent protein. The silent reporter gene can be disposed within a retroviral vector for introduction into the cell line and stable integration. The silent reporter gene can be operably linked to a promoter selected from a viral LTR promoter, a hCMV promoter, a EF1α promoter and a RNA Pol II promoter.

The mRNA inhibitor is an RNAi molecule such as an antisense molecule, an siRNA, a miRNA or a ribozyme. In certain embodiments, the target mRNA comprises one or more of a mRNA encoding HDAC1, daxx or HP1γ. In other embodiments, the target mRNA comprises one or more of a mRNA encoding the gene products of Tables 1 and 2. Inhibition (“knockdown” or “knockout” of a target mRNA), in certain embodiments, is accomplished using one or more mRNA inhibitors targeting the same target mRNA.

The aforementioned method can be adapted to comprise a high-throughput screening system, comprising a plurality of assay chambers in which each assay chamber comprises cells of the cell line into which different mRNA inhibitors are introduced.

Another aspect of the invention features a kit for practicing the above-recited methods. The kit may comprise a container and instructions for practicing the method, and further may comprise one or more of (1) a cell line comprising a genome into which is integrated an epigenetically silent reporter gene; and (2) a mRNA inhibitor. The kit can be adapted for practicing the methods in plurality; for example, it may comprise a plurality of assay containers and a plurality of mRNA inhibitors, and/or it may comprise a multi-well plate, wherein the reporter gene encodes a gene product that is directly or indirectly fluorescently detected.

Another aspect of the invention features a gene product that functions in maintaining epigenetic silencing, selected from HDAC1, Daxx, HP1γ, MBD1, MBD2, MBD3, SETDB1 (also known as ESET or KMT1E), DNMT3A, RING1, PHC2 (also known as HPH2), CHAF1A (also known CAF-1 p150), TRIM24 (also known as TIF1alpha), TRIM33 (also known as TIF1gamma), JMJD2A (also known as KDM4A), SUV420H2 (also known as KMT5C), RAD21, FBXL11 (also known as JHDM1a and KDM2A), PBRM1 (also known as BAF180) and ZMYND8. In one embodiment, the gene product is selected from MBD1, MBD2, MBD3, SETDB1 (also known as ESET or KMT1E) and DNMT3A. Alternatively, the gene product is selected from RING1, PHC2 (also known as HPH2) and CHAF1A (also known CAF-1 p150).

Specifically, the gene product is selected from TRIM24 (also known as TIF1alpha), TRIM33 (also known as TIF1gamma), JMJD2A (also known as KDM4A) and SUV420H2 (also known as KMT5C). Or, specifically, the gene product is selected from RAD21 and FBXL11 (also known as JHDM1a and KDM2A). Or, specifically, the gene product is selected from PBRM1 (also known as BAF180) and ZMYND8.

Another aspect of the invention features a method of relieving epigenetic silencing in a cell, comprising inhibiting production or expression of one or more mRNA molecule in the cell, selected from mRNA encoding HDAC1, Daxx, HP1γ, MBD1, MBD2, MBD3, SETDB1 (also known as ESET or KMT1E), DNMT3A, RING1, PHC2 (also known as HPH2), CHAF1A (also known CAF-1 p150), TRIM24 (also known as TIF1alpha), TRIM33 (also known as TIF1gamma), JMJD2A (also known as KDM4A), SUV420H2 (also known as KMT5C), RAD21, FBXL11 (also known as JHDM1a and KDM2A), PBRM1 (also known as BAF180) and ZMYND8, the inhibition resulting in relief of epigenetic silencing in the cell.

Other features and advantages of the present invention will be understood by reference to the detailed description and examples that follow.

### BRIEF DESCRIPTION OF THE DRAWINGS

FIG. 1 depicts the results of an example experiment demonstrating that the knockdown of HDAC1 or Daxx results in reactivation of the silent GFP reporter. (A) HeLa TI-C cells (Katz et al., 2007, J. Virol. 81:2592-2604) were transfected with the indicated siRNA SMARTpools (100 nM) (Dharmacon) and incubated for 96 hours. GFP expression was analyzed by FACS. NT, not transfected. (B) Histograms of GFP intensities (x axis) from the experiment shown in panel A. Percentages of GFP-positive cells are indicated by the numerical values. Autoscaling was used to portray the distribution of GFP intensities.

FIG. 2 depicts the results of an example experiment demonstrating the determination of specificity of siRNA knockdown. (A) qRT-PCR analysis of target mRNAs. For each target mRNA measurement, the values were normalized to a control which was treated with transfection reagent only (DharmaFECT 1 [DF1]). For HDAC1 and Daxx siRNA treatments, the levels of HDAC1, HDAC2, HDAC3, HDAC4, and Daxx mRNAs were measured. For HDAC2, HDAC3, and HDAC4 siRNAs, only the cognate target mRNA levels were measured (*). (B) Assessment of knockdown by Western blotting. TI-C cells were treated with 100 nM of siRNAs indicated above the panel and cells were processed for Western blotting after 72 hours. GAPDH antibody was used to monitor recovery. Mock siRNA treatments were performed in duplicate. (C) Transfection of a plasmid encoding an siRNA-resistant form of HDAC1 mRNA. Silent mutations that destroy the HDAC1 siRNA 01 annealing site were introduced into an HDAC1 expression plasmid, as described in Materials and Methods. Mutant plasmids prepared in duplicate (R1, R2) or a wt control plasmid was introduced into TI-C cells along with the HDAC1 siRNA 01. GFP expression was monitored by FACS. As shown, HDAC 01 siRNA was capable of stimulating GFP reactivation after transfection of the wt HDAC1 plasmid. In contrast, the siRNA-resistant plasmids were able to repress GFP expression in the presence of the siRNA. (D) Localization of Daxx at the GFP promoter. ChIP analysis was carried out as described in Materials and Methods. Two primer sets were used, targeting the silent viral GFP promoter region or the active cellular β-actin gene. Experiments shown are representative, and the Daxx results are averages of triplicate immunoprecipitations. IgG, immunoglobulin G.

FIG. 3 depicts the results of an example experiment demonstrating that the knockdown of several candidate proteins or treatment with various control siRNAs fails to reactivate the silent GFP reporter. (A and B) HeLa TI-C cells were treated with the indicated siRNAs and the percentages of GFP-positive cells were determined by FACS at 96 hours posttransfection. Single siRNAs were used for H3.3A, H3.3B, and HIRA. Two independent single siRNAs (designated a and b) were tested for HIRA. DF1, DharmaFECT 1 transfection reagent. (C) TI-C cells were treated with 100 nM siRNAs as indicated above the panels and cells were processed for Western blotting after 72 hours. (D) Treatment and analysis with the indicated siRNAs was as for panel A. Negative control siRNAs RISC−, RISC+, and GAPDH were analyzed. (E) The HDAC1 siRNA SMARTpool was titrated to determine the lowest effective concentration versus the negative control siRNA RISC⁺. Analysis was as for panel A.

FIG. 4 depicts the results of an example experiment evaluating TI-C silent cell clones. (A) Clones were treated with TSA or a dimethyl sulfoxide (DMSO) control, and GFP was monitored by FACS after 24 hours. (B) Cell clones were transfected with the indicated siRNAs, and GFP reactivation was monitored by FACS after 96 hours. Representative results are shown.

FIG. 5 depicts the results of an example experiment evaluating the role of HP1 isoforms in silencing maintenance. (A) HeLa TI-C cells were transfected with the indicated HP1 isoform siRNA SMARTpools, and GFP reactivation was monitored by FACS analysis after 96 hours. Abbreviations: NT, not transfected; DF1, DharmaFECT 1 transfection reagent. (B and C) Western blot analyses of siRNA knockdown of the HP1 family of proteins. Cells were transfected with the siRNAs indicated above the panels. The detection of HP1α required loading of 10-fold more protein.

FIG. 6 depicts the results of an example experiment demonstrating that the expression of a dnHP1 reactivates silent GFP. (A) Map of dnHP1. Chromo, chromodomain. (B) A retroviral vector encoding a dnHP1 was used to infect HeLa TI-C cells. Cells were placed under selection with puromycin and were monitored for reactivation of GFP expression by FACS. The FACS profile obtained at 5 days postinfection shows GFP expression in cells selected with the dnHP1 expression vector (no fill) versus the empty vector (filled). The expression of dnHP1 produced a population of GFP-positive cells (24%), some of which were very bright and appear off scale in the graph. A representative experiment is shown.

FIG. 7 depicts the results of an example experiment demonstrating the reactivation of silent GFP by viral proteins. (A and B) HeLa TI-C cells were transfected with expression plasmids encoding the indicated proteins and GFP reactivation was monitored after 48 hours by FACS analysis.

FIG. 8 depicts the results of an example experiment evaluating HeLa cells that harboring silent GFP under the control of the ASV LTR. (A and B) TI-L cells were transfected with the indicated siRNA SMARTpools and GFP expression was measured after 96 hours by FACS analysis. NT, not transfected; two independent single siRNAs tested for HIRA are designated HIRA a and b.

FIG. 9 depicts the results of an example experiment demonstrating the results of a screen of GFP-silent reporter cells with epigenetic pre-selected siRNA set (Table 1). A. Response to siRNAs is measured by percent GFP positive cells. Samples are ranked according to signal strength. Duplicate assays were carried out in 96-well plates and error bars are shown. The initial screen was carried out with two siRNAs for each target (results with one siRNA are shown and error bars are shown). Sample Group 1 signifies activation of GFP in >20% of cells (dashed red line). B. Two sample groups from Panel A are shown as an expanded view. Group 1 highlights the strongest signals and gene target names are indicated for several siRNAs. Other validated gene hits including MBD3, Ring1, HPH2, DNMT3A, and JHDM1b are not indicated in the figure for simplicity (see FIGS. 12-15). Group 2 highlights non-hits.

FIG. 10 depicts the interrelationship among factors identified using the pre-selected epigenetics siRNA set. Protein families are indicated, and specific family members identified in this functional screen are identified in parentheses. A role for an MBD protein (FIG. 12) was identified, but this adapter protein family is not depicted in this figure.

FIG. 11 depicts the results of an example experiment identifying histone methyltransferase activity that maintains epigenetic silencing. A. Results from interrogation with siRNAs targeting H3K9 methyltransferases. Data is extracted from the screen shown in FIG. 9. Shown are results with two independent siRNAs (red, blue), analyzed in duplicate (error bars). Indicated genes represent full coverage of the H3K9 HMT enzyme family, based on current knowledge. An exclusive role for SETDB1 was detected. B. Results in Panel A support roles for H3K9 methylation and possibly HP1 in silencing. ChIP analyses with carried out using standard methods. Gel is shown on left and independent quantitation is shown on the right. Both H3K9 methylation and HP1γ were detected at the silent GFP promoter, consistent with the screen results (FIG. 10).

FIG. 12 depicts the results of example experiments validating the hits and non-hits using four independent siRNAs (Qiagen) and secondary screens. Assays were carried out as described in FIG. 8. Hits (*) are defined as >20% reactivation by at least two independent siRNAs (2/4). Results show independent siRNAs producing >20% reactivation: SETDB1 (4/4), CHAF1A (4/4), MBD3 (3/4), DNMT3A (2/4) and SETDB2 (0/4, also see FIG. 11). Two secondary screens were carried out to measure false negative hits caused by nonspecific cytotoxicity (Alamar blue) or interference with GFP reporter by siRNAs. Arrow indicates the single siRNA in this set that interfered with GFP expression.

FIG. 13 depicts a summary of results with siRNAs targeting histone methyltransferases (above) and histone demethylases (below) that modify the N-terminus of histone H3 (sequence shown). Results were extracted from screen data depicted in FIG. 9A. Red circles indicate non-hits and green circles indicates hits. See FIGS. 11 and 12 for results with the SETDB1 siRNAs.

FIG. 14 depicts a summary of results with siRNAs targeting DNA methyltransferases. Results were collected from screen depicted in FIG. 9. Shown are results with two independent siRNAs (red, blue) (Qiagen) analyzed in duplicate (error bars). Indicated genes represent full coverage of the enzyme family, based on current knowledge. An exclusive role for DNMT3A was detected.

FIG. 15 depicts the results of an example experiment demonstrating the interrogation with siRNAs targeting HPH2 and Ring1, as compared with positive and negative controls (HDAC1 and GAPDH, respectively). Shown are results with four independent siRNAs for HPH2 and Ring1, analyzed in duplicate (error bars). Below are shown sample GFP intensity profiles from 96-well FACS analysis. Profiles shown correspond to samples indicated by arrows.

### DETAILED DESCRIPTION

### Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art. The techniques and procedures are generally performed according to conventional methods in the art and various general references (e.g., Sambrook and Russell, 2001, Molecular Cloning, A Laboratory Approach, Cold Spring Harbor Press, Cold Spring Harbor, N.Y., and Ausubel et al., 2008, Current Protocols in Molecular Biology, John Wiley & Sons, NY), which are provided throughout this document.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “complementary” (or “complementarity”) refers to the specific base pairing of nucleotide bases in nucleic acids. The term “perfect complementarity” as used herein refers to complete (100%) complementarity within a contiguous region of double stranded nucleic acid, such as between a hexamer or heptamer seed sequence in an miRNA and its complementary sequence in a target polynucleotide, as described in greater detail herein.

An “antisense nucleic acid” (or “antisense oligonucleotide”) is a nucleic acid molecule (RNA or DNA) which is complementary to an mRNA transcript or a selected portion thereof. Antisense nucleic acids are designed to hybridize with the transcript and, by a variety of different mechanisms, prevent if from being translated into a protein; e.g., by blocking translation or by recruiting nucleic acid-degrading enzymes to the target mRNA.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Effective amount” or “therapeutically effective amount” are used interchangeably herein, and refer to an amount of a compound, formulation, material, or composition, as described herein effective to achieve a particular biological result.

As used herein “endogenous” refers to any material from or produced inside an organism, cell, tissue or system. “Exogenous” refers to any material introduced from or produced outside an organism, cell, tissue or system.

The term “expression” as used herein is defined as the transcription and/or translation of a particular nucleotide sequence driven by its promoter.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

“Homologous, homology” or “identical, identity” as used herein, refer to comparisons among amino acid and nucleic acid sequences. When referring to nucleic acid molecules, “homology,” “identity,” or “percent identical” refers to the percent of the nucleotides of the subject nucleic acid sequence that have been matched to identical nucleotides by a sequence analysis program. Homology can be readily calculated by known methods. Nucleic acid sequences and amino acid sequences can be compared using computer programs that align the similar sequences of the nucleic or amino acids and thus define the differences. In preferred methodologies, the BLAST programs (NCBI) and parameters used therein are employed, and the DNAstar system (Madison, Wis.) is used to align sequence fragments of genomic DNA sequences. However, equivalent alignment assessments can be obtained through the use of any standard alignment software.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. Unless it is particularly specified otherwise herein, the proteins, virion complexes, antibodies and other biological molecules forming the subject matter of the present invention are isolated, or can be isolated.

The term, “miRNA” or “microRNA” is used herein in accordance with its ordinary meaning in the art. miRNAs are single-stranded RNA molecules of about 20-24 nucleotides, although shorter or longer miRNAs, e.g., between 18 and 26 nucleotides in length, have been reported. miRNAs are encoded by genes that are transcribed from DNA but not translated into protein (non-coding RNA), although some miRNAs are coded by sequences that overlap protein-coding genes. miRNAs are processed from primary transcripts known as pri-miRNA to short stem-loop structures called pre-miRNA and finally to functional miRNA. Mature miRNA molecules are partially complementary to one or more messenger RNA (mRNA) molecules, and they function to regulate gene expression.

The term “operably linked” or “operably inserted” means that the regulatory sequences necessary for expression of the coding sequence are placed in a nucleic acid molecule in the appropriate positions relative to the coding sequence so as to enable expression of the coding sequence. This same definition is sometimes applied to the arrangement other transcription control elements (e.g. enhancers) in an expression vector.

Transcriptional and translational control sequences are DNA regulatory sequences, such as promoters, enhancers, polyadenylation signals, terminators, and the like, that provide for the expression of a coding sequence in a host cell.

The terms “promoter,” “promoter region” or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning and amplification technology, and the like, and by synthetic means. An “oligonucleotide” as used herein refers to a short polynucleotide, typically less than 100 bases in length.

The term “reporter gene” refers to a gene that encodes a product which is easily detectable by standard methods, either directly or indirectly (referred to herein as a “detectable gene product”). The term “silent reporter gene” or “epigenetically silent reporter gene” refers to a reporter gene within a cell that is not expressed due to epigenetic silencing.

The term “RNAi” or “RNA interference” as used herein refers broadly to methods for inhibiting the production of proteins by blocking protein expression with complementary RNA sequences; in other words, a sequence-specific gene silencing technology. Thus, as used herein, the term “RNAi” would include the use of antisense nucleic acids, polynucleotides, siRNA, miRNAs and ribozymes. A molecule used in RNAi is referred to herein as an RNA inhibitor, and encompasses any sequence-specific inhibitory molecule, including antisense nucleic acids, polynucleotides, siRNA, miRNAs and ribozymes.

The term “siRNA” (also “short interfering RNA” or “small interfering RNA”) is given its ordinary meaning, and refers to small strands of RNA (21-23 nucleotides) that interfere with the translation of messenger RNA in a sequence-specific manner. SiRNA binds to the complementary portion of the target messenger RNA and is believed to tag it for degradation. This function is distinguished from that of miRNA, which is believed to repress translation of mRNA but not to specify its degradation.

A cell has been “transformed,” “transduced” or “transfected” by exogenous or heterologous DNA when such DNA has been introduced inside the cell. The introduced DNA may or may not be integrated (covalently linked) into the genome of the cell. In prokaryotes, yeast, and mammalian cells for example, the introduced DNA may be maintained on an episomal element such as a plasmid. With respect to eukaryotic cells, a stably transformed or transduced cell is one in which the introduced DNA has become integrated into a chromosome so that it is inherited by daughter cells through chromosome replication. This stability is demonstrated by the ability of the eukaryotic cell to establish cell lines or clones comprised of a population of daughter cells containing the introduced DNA. A “clone” is a population of cells derived from a single cell or common ancestor by mitosis. A “cell line” is a clone of a primary cell that is capable of stable growth in vitro for many generations.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

A “vector” is a replicon, such as plasmids, phagemids, cosmids, baculoviruses, bacmids, bacterial artificial chromosomes (BACs), yeast artificial chromosomes (YACs), as well as other bacterial, yeast and viral vectors, to which another nucleic acid segment may be operably inserted so as to bring about the replication or expression of the segment. “Expression vector” refers to a vector comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description:

In accordance with one aspect of the invention, a high throughput RNAi-based screen was developed, formulated on the validated principle that knockdown of factors that maintain epigenetic silencing will result in reactivation of a silent reporter gene in cells. RNAi-based screening of these silent reporter cells has identified known enzymes that place or remove epigenetic marks on histones, as well as non-enzymatic proteins that function in silencing or in transfer of marks during S-phase. In addition, in accordance with another aspect of the invention, the screen has been used to identify a number of novel gene products involved in epigenetic silencing, as described in greater detail herein.

The assay system of the invention utilizes a functional readout. Specifically, reporter cells were derived that harbor epigenetically silent reporter genes. Interrogation of these cells with target-specific RNA inhibitors identifies cellular proteins that are involved in, or responsible for, maintaining epigenetic silencing. Using a validated high throughput assay, selected genes or all known human genes can be assayed for their role in epigenetic silencing. The assay of the invention has several advantages: i) it is minimally biased, ii) it measures functions that fit the strict definition of “epigenetic,” iii) it can detect enzymatic or non-enzymatic regulators, iv) it can measure epigenetic regulation of a wide range of promoters at different chromosomal locations, and iv) the assay is validated.

The assay includes the following general steps: (a) providing a cell line comprising a genome into which is integrated an epigenetically silent reporter gene; (b) providing a mRNA inhibitor capable of inhibiting expression of a target mRNA; (c) introducing the mRNA inhibitor into the cell line, thereby inhibiting expression of the target RNA; and (d) detecting an increase in expression of the reporter gene. The increase in expression of the reporter gene is a measure of relief of epigenetic silencing, and indicates therefore that the target mRNA is involved in the epigenetic silencing.

Any transformable or transducible cell that can be maintained in culture can be used in the assay system. Indeed, in certain instances it may be advantageous to use different cell types, e.g., different types of cancer cells, for the purpose of identifying cell type-specific silencing regulators or mechanisms. In one embodiment, the cells lines are of human origin, while in another embodiment they originate with another species. In an exemplary embodiment, human HeLa cells are utilized.

A reporter gene is introduced into the selected cell line and clones containing epigenetically silent reporter genes are selected, as described in greater detail below. The reporter gene can be one that produces any detectable gene product, as is well understood in the art. In particular embodiments, reporter genes that encode spectrophotometrically or fluorescently detectable gene products (either directly colored or fluorescent, or able to produce a colored or fluorescent product) are utilized. Such detectable products include, but are not limited to, b-galactosidase, b-glucuronidase or green fluorescent protein (GFP), the latter of which is exemplified herein.

In accordance with known methods, the reporter gene is operably linked to appropriate expression elements, including promoters. Promoters may include viral promoters or cellular promoters. Nonlimiting examples include viral LTR promoters, hCMV promoters, EF1α promoters and RNA Pol II promoters.

The reporter gene can be introduced into the cell in accordance with any method known in the art. Preferably, the gene should be introduced into the cells in a manner enabling its stable integration into the genome. Certain embodiments of the invention utilize viral vectors for introduction of the reporter gene into the cells. Retroviral vectors are particularly suitable for this purpose. Among the retroviral vectors, alpharetroviruses are particularly suitable because they are known to be silenced at high frequency in mammalian cells. In an exemplary embodiment, avian sarcoma virus (ASV) vectors are utilized. The vectors utilized in the assay system should be able totransduce, but not spread in cells. As such, reporter-expressing cells represent only initial transductants and corresponding generations of daughter cells.

Methods of making cells that contain silent reporter genes have been described in the art, e.g., by Katz et al., 2007, J. Virol. 81:2592-2604. Briefly, cells are transduced with a vector comprising the reporter gene, then sorted for cells that are phenotypically silent for reporter gene expression, yet contain suitable levels of integrated reporter DNA. To assess whether the silent reporter genes can be reactivated, the silent reporter cell population can be treated with a known inhibitor of expression repressors, e.g., trichostatin A (TSA), which block the repressive histone deacetylases (HDACS), thereby relieving epigenetic silencing and activating gene expression. By carrying out repeated rounds of expression activation and silencing, a population of cells enriched in silent reporters can be obtained, and ultimately yield a pure population and clones of cells harboring silent reporter genes. Once established, this reporter-silent phenotype can be maintained during long term passage of the cells.

The central premise of the assay system is that knockdown of key factors that maintain silencing is predicted to cause reactivation of the silent reporter gene, and this can be monitored in a high throughput setting using the detectable gene product as a readout. By utilizing an RNAi-based approach, the assay can be used on a gene-by-gene basis to screen selected candidate genes (as described in detail in Example 1), or to explore various classes of genes (as described in detail in Example 3), or to canvas an entire genome.

RNAi molecules suitable for targeting selected mRNAs include any type of molecule capable of recognizing its target mRNA and interfering with the function of that mRNA. Such interference may comprise degradation of the mRNA (directly or indirectly), interference with translation, or any of a variety of other mechanisms. In one embodiment, the RNAi molecules are siRNA. In other embodiments, the RNAi molecules are miRNA or antisense nucleic acids. In some embodiments, a single mRNA inhibitor (e.g., a single siRNA) per target is employed in an assay. In other embodiments, multiple RNAi molecules (e.g., 2, 3, 4 or more) directed to a single mRNA target are employed.

In preferred embodiments, multiple targets are screened in parallel to generate a high throughput assay, in accordance with methods known in the art. For example, parallel assays may be carried out in a multi-well plate, such as a 96-well plate. An example of a 96-well plate high-throughput assay protocol is set forth in Example 2. Variations will be apparent to the skilled artisan.

As mentioned above, the effect of RNAi-mediated mRNA knockdown on epigenetic silencing is determined by measuring an increase in expression of the silent reporter gene in the cells. Any suitable detection means may be used for this purpose. In certain embodiments, the detectable gene product (or a product thereof) is spectrophotometrically or fluorescently detectable. In an exemplified embodiment, the detectable product is GFP and may be detected by way of its fluorescence. In the high throughput assay described in Example 2, GFP is detected using 96-well FACS (fluorescence activated cell sorting) instrument. A fluorescence plate reader may also be utilized.

Components of the assay systems described above may be conveniently packaged in kits. Such kits may contain, for example, various reagents for individual assays and instructions for their use. Typical kit components include, for example, (1) a cell line comprising a genome into which is integrated an epigenetically silent reporter gene; (2) one or more RNAi molecules targeted to selected mRNA targets; (3) reagents for introducing the RNAi molecules into the cells; and (4) positive and negative controls, which may be RNAi or which may be chemical inhibitors of known epigenetic silencing gene products.

The assays of the present invention have been utilized to identify a number of mRNA targets whose gene products are involved in epigenetic silencing. Example 1 describes the identification and validation of three gene products: knockdown of mRNA encoding HDAC1, the transcriptional repressor Daxx (a binding partner of HDAC1), or heterochromatin protein 1 gamma (HP1γ) resulted in robust and specific GFP reporter gene reactivation. Examples 3 and 4 describe the identification of sixteen hits from a pool of 189 target mRNAs (see Table I). These include (1) MBD1, (2) MBD2, (3) MBD3, (4) SETDB1 (also known as ESET or KMT1E), (5) DNMT3A, (6) RING1, (7) PHC2 (also known as HPH2), (8) CHAF1A (also known CAF-1 p150), (9) TRIM24 (also known as TIF1alpha), (10) TRIM33 (also known as TIF1 gamma), (11) JMJD2A (also known as KDM4A), (12) SUV420H2 (also known as KMT5C), (13) RAD21, (14) FBXL11 (also known as JHDM1a and KDM2A), (15) PBRM1 (also known as BAF180) and (16) ZMYND8.

Of the foregoing gene products, MBD1, MBD2, MBD3, SETDB1 (also known as ESET or KMT1E and DNMT3A) might have been predicted by way of their known functionality. The following two groups were less predictable: (1) RING1, PHC2 (also known as HPH2) and CHAF1A (also known CAF-1 p150); and (2) TRIM24 (also known as TIF1alpha), TRIM33 (also known as TIF1gamma), JMJD2A, KDM4A, SUV420H2 and KMT5C. The following additional two groups appear to represent novel and/or unexpected participants in epigenetic silencing: (1) RAD21, FBXL11 (also known as JHDM1a) and KDM2A; and (2) PBRM1 (also known as BAF180) and ZMYND8. Eight of the sixteen gene products identified by the 189-target screen of Example 3 are discussed in greater detail in Example 4.

Inhibitors of one or more of the gene products described above may be used to relieve epigenetic silencing in a cell. Such methods comprise introducing selected inhibitors, i.e., RNAi molecules into cells, whereupon inhibition of the target mRNA results in partial or full relief of epigenetic silencing and expression of some or all of the previously silenced genes in the cell. Such methods can be tailored to relieve certain types of silencing, for instance, by targeting certain classes of mRNAs whose gene products are involved in silencing of particular genes or particular classes of genes, or by targeting certain cell types.

The identification of gene products that participate in epigenetic silencing has significant relevance to disease, particularly cancer and other proliferative disease. Epigenetic alterations associated with cancer include histone hypoacetylation and DNA hypermethylation. Cancer cells display an imbalance in DNA methylation, characterized by global hypomethylation and hypermethylation of CpG islands. Frequently, expression of genes involved in cell cycle regulation and DNA repair are affected by these alterations in DNA methylation patterns. It is believed that inappropriate epigenetic silencing of growth control genes can be a key step in tumorogenesis. This theory is a logical extension of the tumor suppressor gene hypothesis whereby genetic mutations and deletions inactivate allelic copies of growth control genes. In terms of relevance to cancer, it is now appreciated that epigenetic effects contribute at a frequency comparable to genetic mutations. There is a significant distinction between genetic and epigenetic etiologies of cancer with respect to potential therapeutic approaches. Once genetic mutations (i.e. point mutations and rearrangements) become fixed in the genome, they are essentially irreparable. In contrast, the epigenetic marks that mediate gene silencing display significant plasticity. As such, there is great therapeutic potential in devising strategies that can relieve inappropriate epigenetic silencing. Currently, both DNMT and HDAC inhibitors are currently in use for cancer therapy. With respect to the therapeutic effects of these compounds, it is believed that their mechanism of action includes the reactivation of silent tumor suppressor genes leading to tumor cell differentiation, apoptosis, or cell cycle arrest. The efficacy of these compounds is clearly dependent on increased sensitivity of cancer cells versus normal cells. Such compounds have now been developed as clinical agents, although their mechanism of action is not well understood. Much of the uncertainty comes from the fact that HDACs and DNMTs comprise families of enzymes, and HDACs can recognize a variety of non-histone substrates. Furthermore most HDAC inhibitors have broad activity among HDAC family members. Recent studies indicate that inhibition of individual HDACs using siRNA may have potential therapeutic effects at the cellular level (Senese, et al., 2007, Mol. Cell. Biol. 27:4784-4795). Likewise, the several gene products listed above represent a wealth of additional targets for the development of cancer therapeutic agents.

### Nucleic Acid Inhibitors

Nucleic acid inhibitors according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical modifications thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex and hybrid states. By way of non-limiting examples, nucleic acids useful in the invention include sense nucleic acids, antisense nucleic acids, polynucleotides, siRNA, miRNAs and ribozymes.

In various embodiments of the invention, the nucleic acids sharing all or some portion of the sequences described herein, can be administered to a subject to diminish the level of epigenetic silencing. By way of non-limiting examples, nucleic acid reference sequences, upon which the sequences of the nucleic acid inhibitors of the invention can be based, include, but are not limited to those listed in Tables 1 and 2 and/or exemplified by SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 836, 837, 838, 839, 840, 841 and 842.

It will be readily understood by one skilled in the art that nucleic acid sequences useful in the methods of the invention, include not only the nucleic acid reference sequences provided herein as examples, but also include fragments, modifications and variants, as elsewhere defined herein, of the example nucleic acid reference sequences provided herein.

### Anti-Sense Nucleic Acids

In one embodiment of the invention, an antisense nucleic acid sequence, which may be expressed by a vector, is used to relieve epigenetic silencing. The antisense expression vector can be used to transfect or infect a cell or the mammal itself, thereby causing reduced epigenetic silencing.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule and thereby inhibiting expression of the mRNA.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 50, and more preferably about 20 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

In various embodiments of the invention, antisense nucleic acids with sequences corresponding to all or some portion of the sequences exemplified herein by SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 836, 837, 838, 839, 840, 841 and 842, can be administered to an individual to diminish the level of epigenetic silencing.

### Ribozymes

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

In various embodiments of the invention, ribozymes that specifically cleave a sequence exemplified at least one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 836, 837, 838, 839, 840, 841 and 842, can be administered to a subject to diminish the level of epigenetic silencing.
siRNA

In one embodiment, siRNA is used to decrease the level of epigenetic silencing. RNA interference (RNAi) is a phenomenon in which the introduction of double-stranded RNA (dsRNA) into a diverse range of organisms and cell types, causes degradation of the complementary mRNA. Generally, in the cell, long dsRNAs are cleaved into shorter 21-25 nucleotide small interfering RNAs, or siRNAs, by a ribonuclease (e.g., Dicer). The siRNAs subsequently assemble with protein components into an RNA-induced silencing complex (RISC), unwinding in the process. Activated RISC then binds to a complementary transcript by base pairing interactions between the siRNA antisense strand and the mRNA. The bound mRNA is cleaved and sequence specific degradation of mRNA results in the diminution of the gene products. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, Pa. (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of protein using RNAi technology.

### Modification of Nucleic Acids

Following the generation of the nucleic acid inhibitors of the present invention, a skilled artisan will understand that the nucleic acid will have certain characteristics that can be modified to improve the nucleic acid as a therapeutic compound. Therefore, the nucleic acid may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987 Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any nucleic acid of the invention may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl-methyl-, thio- and other modified forms of adenine, cytidine, guanine, thymine, and uridine.

### Vectors

In other related aspects, the invention includes an isolated nucleic acid encoding an nucleic acid inhibitor, such as, for example, an antisense nucleic acid, a polynucleotide, a ribozyme, an miRNA or an siRNA, wherein the isolated nucleic acid encoding the nucleic acid inhibitor is operably linked to a nucleic acid comprising a promoter/regulatory sequence. Thus, the invention encompasses expression vectors and methods for the introduction of exogenous DNA into cells with concomitant expression of the exogenous DNA in the cells such as those described, for example, in Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

In another aspect, the invention includes a vector comprising an siRNA polynucleotide. Preferably, the siRNA polynucleotide is capable of inhibiting the expression of a target mRNA, such as one listed in Tables 1 or 2, and/or exemplified by SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 836, 837, 838, 839, 840, 841 and 842. The incorporation of a desired nucleic acid into a vector and the choice of vectors is well-known in the art as described in, for example, Sambrook et al., supra, and Ausubel et al., supra.

The nucleic acid inhibitor can be cloned into a number of types of vectors. However, the present invention should not be construed to be limited to any particular vector. Instead, the present invention should be construed to encompass a wide plethora of vectors which are readily available and/or well-known in the art. For example, nucleic acid inhibitor of the invention can be cloned into a vector including, but not limited to a plasmid, a phagemid, a phage derivative, an animal virus and a cosmid. Vectors of particular interest include expression vectors, replication vectors, probe generation vectors and sequencing vectors.

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides. Many such systems are commercially and widely available.

Further, the expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

For expression of the nucleic acid inhibitor of the invention, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. No. 4,683,202, U.S. Pat. No. 5,928,906). Furthermore, it is contemplated that control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle and organism chosen for expression. Those of skill in the art of molecular biology generally know how to use promoters, enhancers and cell type combinations for protein expression, for example, see Sambrook et al. (2001). The promoters employed may be constitutive, tissue-specific, inducible and/or useful under the appropriate conditions to direct high level expression of the introduced DNA segment, such as is advantageous in the large-scale production of recombinant proteins and/or peptides. The promoter may be heterologous or endogenous.

A promoter sequence exemplified in the experimental examples presented herein is the cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue specific promoter, which promoter is active only in a desired tissue. Tissue specific promoters are well known in the art and include, but are not limited to, the HER-2 promoter and the PSA associated promoter sequences.

In order to assess the expression of the nucleic acid inhibitor of the invention, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

### Methods of Administration

The methods of the invention comprise administering a therapeutically effective amount of at least one nucleic acid, having a sequence exemplified by at least one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 836, 837, 838, 839, 840, 841 and 842, to a subject, where the nucleic acid reduces, diminishes or decreases the level of epigenetic silencing. In a preferred embodiment the subject is a mammal. In a more preferred embodiment the subject is a human.

Decreasing the level of expression of a gene production, such as a mRNA exemplified by at least one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 836, 837, 838, 839, 840, 841 and 842, includes decreasing the half-life or stability of the mRNA, decreasing the level of translation of the mRNA, or decreasing the level of the polypeptide exemplified by at least one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832 and 834. Methods of decreasing expression of mRNA include, but are not limited to, methods that use an siRNA, a miRNA, an antisense nucleic acid, a ribozyme, a polynucleotide or other specific inhibitors of mRNA, as well as combinations thereof.

The present invention should in no way be construed to be limited to the inhibitors described herein, but rather should be construed to encompass any inhibitor of any one of the mRNAs exemplified by SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 836, 837, 838, 839, 840, 841 and 842, both known and unknown, that diminishes and reduces targent RNA expression and/or that diminishes and reduces epigenetic silencing.

The methods of the invention comprise administering a therapeutically effective amount of at least one nucleic acid inhibitor to a mammal wherein a nucleic acid inhibitor, or combination thereof prevents, attenuates, reduces or diminishes targent mRNA expression and/or that prevents, attenuates, reduces or diminishes epigenetic silencing.

The method of the invention comprises administering a therapeutically effective amount of at least one nucleic acid inhibitor to a subject wherein a composition of the present invention comprising a nucleic acid inhibitor, or a combination thereof is used either alone or in combination with other therapeutic agents. The invention can be used in combination with other treatment modalities, such as chemotherapy, radiation therapy, and the like. Examples of chemotherapeutic agents that can be used in combination with the methods of the invention include, for example, carboplatin, paclitaxel, and docetaxel, cisplatin, doxorubicin, and topotecan, as well as others chemotherapeutic agents useful as a combination therapy that may discovered in the future.

Isolated nucleic acid-based inhibitors can be delivered to a cell in vitro or in vivo using vectors comprising one or more isolated nucleic acid inhibitor sequences. In some embodiments, the nucleic acid sequence has been incorporated into the genome of the vector. The vector comprising an isolated nucleic acid inhibitor described herein can be contacted with a cell in vitro or in vivo and infection or transfection can occur. The cell can then be used experimentally to study, for example, the effect of an isolated nucleic acid inhibitor in vitro. The cell can be migratory or non-migratory. The cell can be present in a biological sample obtained from the subject (e.g., blood, bone marrow, tissue, fluids, organs, etc.) and used in the treatment of disease, or can be obtained from cell culture.

Various vectors can be used to introduce an isolated nucleic acid inhibitor into mammalian cells. Examples of viral vectors have been discussed elsewhere herein and include retrovirus, adenovirus, parvovirus (e.g., adeno-associated viruses), coronavirus, negative-strand RNA viruses such as orthomyxovirus (e.g., influenza virus), rhabdovirus (e.g., rabies and vesicular stomatitis virus), paramyxovirus (e.g. measles and Sendai), positive-strand RNA viruses such as picornavirus and alphavirus, and double stranded DNA viruses including adenovirus, herpesvirus (e.g., herpes simplex virus types 1 and 2, Epstein-Barr virus, cytomegalovirus), and poxvirus (e.g. vaccinia, fowlpox and canarypox). Other viruses include Norwalk virus, togavirus, flavivirus, reoviruses, papovavirus, hepadnavirus and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus, lentiviruses and baculoviruses.

In addition, an engineered viral vector can be used to deliver an isolated nucleic acid inhibitor of the present invention. These vectors provide a means to introduce nucleic acids into cycling and quiescent cells, and have been modified to reduce cytotoxicity and to improve genetic stability. The preparation and use of engineered Herpes simplex virus type 1 (Krisky et al., 1997, Gene Therapy 4:1120-1125), adenoviral (Amalfitanl et al., 1998, Journal of Virology 72:926-933) attenuated lentiviral (Zufferey et al., 1997, Nature Biotechnology 15:871-875) and adenoviral/retroviral chimeric (Feng et al., 1997, Nature Biotechnology 15:866-870) vectors are known to the skilled artisan. In addition to delivery through the use of vectors, an isolated a nucleic acid inhibitor can be delivered to cells without vectors, e.g. as “naked” nucleic acid delivery using methods known to those of skill in the art. See, for example, U.S. Pat. Nos. 5,350,674 and 5,585,362.

Physical methods for introducing a nucleic acid into a host cell include transfection, calcium phosphate precipitation, lipofection, particle bombardment, microinjection, electroporation and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York), and in Ausubel et al. (2001, Current Protocols in Molecular Biology, John Wiley & Sons, New York).

Chemical means for introducing a nucleic acid into a host cell include colloidal dispersion systems, such as macromolecule complexes, nanocapsules, microspheres, beads, and lipid-based systems including oil-in-water emulsions, micelles, mixed micelles and liposomes. A preferred colloidal system for use as a delivery vehicle in vitro and in vivo is a liposome (i.e., an artificial membrane vesicle). The preparation and use of such systems is well known in the art.

Various forms of an isolated nucleic acid inhibitor, as described herein, can be administered or delivered to a mammalian cell (e.g., by virus, direct injection, or liposomes, or by any other suitable methods known in the art or later developed). The methods of delivery can be modified to target certain cells, and in particular, cell surface receptor molecules. As an example, the use of cationic lipids as a carrier for nucleic acid constructs provides an efficient means of delivering the isolated nucleic acid inhibitor of the present invention.

Various formulations of cationic lipids have been used to deliver nucleic acids to cells (WO 91/17424; WO 91/16024; U.S. Pat. Nos. 4,897,355; 4,946,787; 5,049,386; and 5,208,036). Cationic lipids have also been used to introduce foreign nucleic acids into frog and rat cells in vivo (Holt et al., Neuron 4:203-214 (1990); Hazinski et al., Am. J. Respr. Cell. Mol. Biol. 4:206-209 (1991)). Therefore, cationic lipids may be used, generally, as pharmaceutical carriers to provide biologically active substances (for example, see WO 91/17424; WO 91/16024; and WO 93/03709). Thus, cationic liposomes can provide an efficient carrier for the introduction of nucleic acids into a cell.

Further, liposomes can be used as carriers to deliver a nucleic acid to a cell, tissue or organ. Liposomes comprising neutral or anionic lipids do not generally fuse with the target cell surface, but are taken up phagocytically, and the nucleic acids are subsequently subjected to the degradative enzymes of the lysosomal compartment (Straubinger et al., 1983, Methods Enzymol. 101:512-527; Mannino et al., 1988, Biotechniques 6:682-690). However, as demonstrated by the data disclosed herein, an isolated siRNA of the present invention is a stable nucleic acid, and thus, may not be susceptible to degradative enzymes. Further, despite the fact that the aqueous space of typical liposomes may be too small to accommodate large macromolecules, the isolated nucleic acid inhibitor of the present invention is relatively small, and therefore, liposomes are a suitable delivery vehicle for the present invention. Methods of delivering a nucleic acid to a cell, tissue or organism, including liposome-mediated delivery, are known in the art and are described in, for example, Feigner (Gene Transfer and Expression Protocols Vol. 7, Murray, E. J. Ed., Humana Press, New Jersey, (1991)).

In other related aspects, the invention includes an isolated nucleic acid inhibitor operably linked to a nucleic acid comprising a promoter/regulatory sequence such that the nucleic acid is preferably capable of delivering an isolated nucleic acid inhibitor. Thus, the invention encompasses expression vectors and methods for the introduction of an isolated nucleic acid inhibitor into or to cells.

Such delivery can be accomplished by generating a plasmid, viral, or other type of vector comprising an isolated nucleic acid inhibitor operably linked to a promoter/regulatory sequence which serves to introduce the nucleic acid inhibitor into cells in which the vector is introduced. Many promoter/regulatory sequences useful for the methods of the present invention are available in the art and include, but are not limited to, for example, the cytomegalovirus immediate early promoter enhancer sequence, the SV40 early promoter, as well as the Rous sarcoma virus promoter, and the like. Moreover, inducible and tissue specific expression of an isolated nucleic acid inhibitor may be accomplished by placing an isolated nucleic acid inhibitor, with or without a tag, under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In addition, promoters which are well known in the art which are induced in response to inducing agents such as metals, glucocorticoids, and the like, are also contemplated in the invention. Thus, it will be appreciated that the invention includes the use of any promoter/regulatory sequence, which is either known or unknown, and which is capable of driving expression of the desired protein operably linked thereto.

Selection of any particular plasmid vector or other vector is not a limiting factor in the invention and a wide plethora of vectors are well-known in the art. Further, it is well within the skill of the artisan to choose particular promoter/regulatory sequences and operably link those promoter/regulatory sequences to a DNA sequence encoding a desired polypeptide. Such technology is well known in the art and is described, for example, in Sambrook et al. (2001, Molecular Cloning: A Laboratory Manual, Cold Spring Harbor Laboratory, New York), and in Ausubel et al. (2001, Current Protocols in Molecular Biology, John Wiley & Sons, New York) and elsewhere herein.

### Pharmaceutical Compositions and Therapies

Administration of a nucleic acid inhibitor comprising one or more nucleic acids, antisense nucleic acids, polynucleotides, ribozymes, miRNAs or siRNAs of the invention in a method of treatment can be achieved in a number of different ways, using methods known in the art. Such methods include, but are not limited to, providing exogenous nucleic acids, antisense nucleic acids, polynucleotides, ribozymes, miRNAs or siRNAs to a subject or expressing a recombinant nucleic acid, antisense nucleic acid, polynucleotide, ribozyme, miRNA or siRNA expression cassette.

The therapeutic and prophylactic methods of the invention thus encompass the use of pharmaceutical compositions comprising a nucleic acid inhibitor, antisense nucleic acid, polynucleotide, ribozyme, miRNA or siRNA of the invention. The pharmaceutical compositions useful for practicing the invention may be administered to deliver a dose of between 1 ng/kg/day and 100 mg/kg/day. In one embodiment, the invention envisions administration of a dose which results in a concentration of the compound of the present invention between 1 μM and 10 μM in a mammal. In another embodiment, the invention envisions administration of a dose which results in a concentration of the compound of the present invention between 1 μM and 10 μM in a cell of a mammal.

Typically, dosages which may be administered in a method of the invention to an animal, preferably a human, range in amount from 0.5 μg to about 50 mg per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 μg to about 10 mg per kilogram of body weight of the animal. More preferably, the dosage will vary from about 3 μg to about 1 mg per kilogram of body weight of the animal.

The compound may be administered to an animal as frequently as several times daily, or it may be administered less frequently, such as once a day, once a week, once every two weeks, once a month or even less frequently, such as once every several months or even once a year or less. The frequency of the dose will be readily apparent to the skilled artisan and will depend upon any number of factors, such as, but not limited to, the type and severity of the disease being treated, the type and age of the animal, etc. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. A unit dose is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

Parenteral administration of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, and intratumoral.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

### Kits

The invention also includes a kit comprising a nucleic acid inhibitor, or combinations thereof, of the invention and an instructional material which describes, for instance, administering the nucleic acid inhibitor, or a combinations thereof, to a subject as a therapeutic treatment or a non-treatment use as described elsewhere herein. In an embodiment, this kit further comprises a (preferably sterile) pharmaceutically acceptable carrier suitable for dissolving or suspending the therapeutic composition, comprising a nucleic acid inhibitor, or combinations thereof, of the invention, for instance, prior to administering the molecule to a subject. Optionally, the kit comprises an applicator for administering the inhibitor. A kit providing a nucleic acid, antisense nucleic acid, polynucleotide, ribozyme, miRNA or siRNA of the invention and an instructional material is also provided.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

This example sets forth a demonstration of the efficacy of a small interfering RNA (siRNA)—based method of identifying specific cellular factors that participate in the maintenance of retroviral epigenetic silencing.

The Materials and Methods are now described.

Cells. HeLa cell populations containing silent GFP genes, as described by Katz et al., 2007, J. Virol. 81:2592-2604, were utilized. TI-C cell clones were isolated by cell sorting as described by Katz et al., 2007, J. Virol. 81:2592-2604.

Analysis of GFP expression. GFP expression was quantitated by FACScan as described previously (Greger et al., 2005, J. Virol. 79:4610-4618; Katz et al., 2007, J. Virol. 81:2592-2604; Poleshko et al., 2008, J. Virol. 82:2313-2323).

Western blot analysis. Western blotting was performed by standard methods as described previously (Greger et al., 2005, J. Virol. 79:4610-4618). Anti-HP1α (MAB3446), anti-HP1β (MAB3448), anti-HP1γ (MAB3450), and anti-GAPDH (AP124P) were purchased from Chemicon, Temecula, Calif. Anti-NF-κB p50 (ab7971), anti-Dicer (ab13502), anti-HDAC1 (ab19845), and anti-HDAC2 (ab16032) were purchased from Abcam, Cambridge, Mass. Anti-Daxx (D7810) was purchased from Sigma-Aldrich, St. Louis, Mo. Goat anti-rabbit (31462; Pierce, Rockford, Ill.) and goat anti-mouse (AP124P; Chemicon) peroxidase-conjugated second antibodies and enhanced chemiluminescence reagents (Pierce) were used according to the manufacturers' instructions.

siRNAs. All siRNA SMARTpools and single siDuplexes were purchased from Dharmacon (Lafayette, Colo.). DharmaFECT 1 transfection reagent (T-2001-02) was used according to the manufacturer's protocols. The following siRNAs were used: siCONTROL GAPDH duplex (D-001140-01), siCONTROL RISC-free siRNA #1 (D-001220-01), and siCONTROL nontargeting siRNA #1 (D-001210-01). The following siRNA SMARTpools were used: HP1αα/CBX1 (M-009716-00), HP1β/CBX3 (M-010033-00), HP1γ/CBX5 (M-004296-01), Dicer1 (M-003483-00), HDAC1 (M-003493-02), HDAC2 (M-094936-00), HDAC3 (M-003496-00), HDAC4 (M-003497-02), and Daxx (M-004420-00).

The following single siRNAs were used: NF-κB1 (D-003520-01/02/03/05), Daxx (D-004420-01/02/03/04), HDAC1 (D-003493-01/02/04/09), H3.3A (D-011684-04), H3.3B (D-012051), and HIRA (D-013610-02/04).

Quantitative RT-PCR (qRT-PCR). RNA was quantified using the Agilent 2100 BioAnalyzer in combination with an RNA 6000 Nano LabChip. RNA was reverse transcribed using the Moloney murine leukemia virus reverse transcriptase (RT) (Ambion, Austin Tex.) and a mixture of anchored oligo(dT) and random decamers. Aliquots of cDNAs were used for PCR. Real-time PCR assays were run using an ABI 7900 HT instrument. The primers and probes were designed using Primer Express version 1.5 software from Applied Biosystems and synthesized by the Fox Chase Cancer Center Fannie Rippel Biotechnology Facility. The probes were 5′-6FAM and 3′-BHQ1 labeled. PCR master mix from Eurogentec was used for PCR. Cycling conditions were 95° C. for 15 min followed by 40 (two-step) cycles (95° C. for 15 s and 60° C. for 60 s). PolR2F was used as the reference gene. The 2^{-ΔΔ}_{CT} method (C_{T}, threshold cycle) was used to calculate relative changes in expression. For each sample, the values are averages and standard deviations of data from two PCRs performed with two amounts (100 and 20 ng) of total RNA in the RT reaction. The following primers and probe sequences were used: for Daxx, 5′-AGGGCCATTAGGAAACAGCTA (forward) (SEQ ID NO: 843), 5′-AGGGTACATATCTTTTTCCCATTCTT (reverse) (SEQ ID NO: 844), and 5′-TGGAAAGGCAAAGGTCAGTGCATGA (probe) (SEQ ID NO: 845); for HDAC1, 5′-TGAGGACGAAGACGACCCT (forward) (SEQ ID NO: 846), 5′-CTCACAGGCAATTCGTTTGTC (reverse) (SEQ ID NO: 847), and 5′-CAAGCGCATCTCGATCTGCTCCTC (probe) (SEQ ID NO: 848); for HDAC2, 5′-CTTTCCTGGCACAGGAGACTT (forward) (SEQ ID NO: 849), 5′-CACATTGGAAAATTGACAGCATAGT (reverse) (SEQ ID NO: 850), and 5′-AGGGATATTGGTGCTGGAAAAGGCAA (probe) (SEQ ID NO: 851); for HDAC3, 5′-GCTCCTCACTAATGGCCTCTTC (forward) (SEQ ID NO: 852), 5′-GGTGGTTATACTGTCCGAAATGTT (reverse) (SEQ ID NO: 853), and 5′-AGCAGCGATGTCTCATATGTCCAGCA (probe) (SEQ ID NO: 854); and for HDAC4, 5′-TTGAGCGTGAGCAAGATCCT (forward) (SEQ ID NO: 855), 5′-GACGCTAGGGTCGCTGTAGAA (reverse) (SEQ ID NO: 856), and 5′-CGTGGACTGGGACGTGCACCA (probe) (SEQ ID NO: 857).

dnHP1 expression vector. The construction and use of a retroviral vector encoding a dominant negative form of HP1 (dnHP1) was described previously (HP1βΔN) (Zhang et al., 2007, Mol. Cell. Biol. 27:949-962).

Plasmids and transfection. Immediate early 1 (IE1) and IE2 expression plasmids (Nevels, et al., 2004, Proc. Natl. Acad. Sci. USA 101:17234-17239), Gam1 wild-type (wt) and mutant expression plasmids (Chiocca, et al., 2002, Curr. Biol. 12:594-598), and pp71 wt and mutant expression plasmids were utilized. Transfections were carried out using Lipofectamine or Lipofectamine 2000 (Invitrogen, Carlsbad Calif.) as described by the supplier.

Construction and transfection of an HDAC1 siRNA-resistant expression plasmid. An HDAC1 expression plasmid was purchased from Origene (Rockville, Md.). The QuikChange mutagenesis kit (Stratagene, La Jolla, Calif.) was used to introduce silent changes in the HDAC1 codons to create an siRNA-resistant site for HDAC1 siRNA 01. The oligodeoxynucleotides used for mutagenesis were:

(SEQ ID NO: 858)
5′-GGATACGGAGATCCCTAATGAGCTCCCCTACAATGACTACTTTG-3′
and
(SEQ ID NO: 859)
5′-CAAAGTAGTCATTGTAGGGGAGCTCATTAGGGATCTCCGTATCC-3′.

The wt and resistant plasmids were used to transfect HeLa TI-C cells in the presence of HDAC1 siRNA 01 by use of the Dharmacon DharmaFECT Duo transfection reagent as described by the supplier.

ChIP. Chromatin immunoprecipitation (ChIP) reactions from TI-C cells were performed using the Upstate/Millipore EZ ChIP kit. Anti-Daxx antibody was obtained from Santa Cruz (sc-7152), and the immunoglobulin G negative control antibody was provided with the EZ ChIP kit. For PCRs, 2 μl of purified DNA from precipitated chromatin was amplified by PCR with the following primers: CMV-GFP (positions −306 to +20 surrounding the transcriptional start site) (5′-CTT ATG GGA CTT TCC TAC TTG-3′ [forward] (SEQ ID NO: 860) and 5′-TCC TCG CCC TTG CTC ACC ATG-3′ [reverse] (SEQ ID NO: 861)) and β-actin coding region (positions 68 to 327) (5′-CTC ACC ATG GAT GAT GAT ATC GC-3′ [forward] (SEQ ID NO: 862) and 5′-ATT TTC TCC ATG TCG TCC CAG TTG-3′ [reverse] (SEQ ID NO: 863)). The PCR products were analyzed on 2% agarose gels. For the quantitation of PCR products, gels were stained with Syto 60 (Invitrogen) and analyzed using the Odyssey imaging system (LI-COR).

The Results of the experiments are now described.

Silent retroviruses are reactivated by HDAC1 and Daxx siRNAs. The protocol described herein utilized a previously isolated subset of HeLa cells that contain silent retroviral GFP reporter genes that can be reactivated by treatment with a variety of HDIs, including TSA (Katz et al., 2007, J. Virol. 81:2592-2604). These cells are designated TSA inducible (TI) and additional versions have been derived, which harbor silent GFP retroviral reporter genes under the control of the human cytomegalovirus (hCMV) IE (TI-C) or ASV (TI-L) long terminal repeat (LTR) (TI-L) promoters (Katz et al., 2007, J. Virol. 81:2592-2604). Such cells represented a significant fraction of the infected culture, as the ratio of GFP+ cells to TI-C cells was approximately 2:1 (Katz et al., 2007, J. Virol. 81:2592-2604; Poleshko et al., 2008, J. Virol. 82:2313-2323)

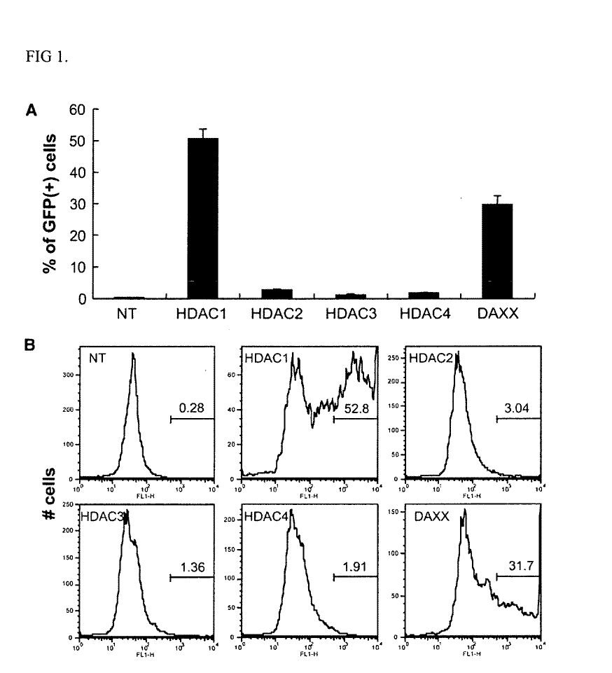

TSA has broad activity against class 1 (widely expressed) and class 2 (primarily tissue-specific) HDACs. HDAC1 and HDAC2 (class 1) were considered to be strong candidates for meditating silencing, as they were detected in complexes with ASV viral DNA in HeLa cells early after infection (Greger et al., 2005, J. Virol. 79:4610-4618). Expression of the class 2 HDAC4 is typically more restricted to specific tissues; however, HDAC4 was previously detected in HeLa cells (Greger et al., 2005, J. Virol. 79:4610-4618). Initial experiments were designed to use siRNAs to identify roles for one or more HDACs in silencing maintenance, as siRNA-mediated knockdown of HDACs might phenocopy HDI activity. TI-C cells were treated with siRNA “SMARTpools” (Dharmacon Corp.) (100 nM) comprising a mixture of four siRNAs that target single mRNAs. Initial tests included siRNAs specific for HDAC1, HDAC2, HDAC3, and HDAC4. As shown in FIG. 1A, treatment of the TI-C population with an siRNA pool that targets HDAC1 resulted in the appearance of a significant fraction of cells that expressed GFP, as measured by fluorescence-activated cell sorter (FACS) analysis. In contrast, siRNA pools that target HDAC2, HDAC3, and HDAC4 mRNA had no significant effect. As shown in the FACS profiles in FIG. 1B, treatment with the HDAC1 siRNA pool resulted in the appearance of cells with very high GFP fluorescence intensities. The HDAC2 siRNA pool consistently produced a small increase in the number of GFP-expressing cells, but the GFP intensity in this small fraction was just above the background level (FIG. 1B). Previous studies suggested a role for Daxx in the initiation of retroviral silencing in this system. Transfection of the TI-C cell population with the Daxx siRNA pool also resulted in robust reactivation of GFP (FIGS. 1A and B), although it was not as pronounced as observed with the HDAC1 siRNA (FIG. 1B).

The TI-C cells used here were previously found to oscillate between responsive and nonresponsive states in terms of reactivation by HDIs (Katz et al., 2007, J. Virol. 81:2592-2604). It is likely that this phenomenon also contributes to the incomplete reactivation observed with HDAC1 and Daxx siRNAs.

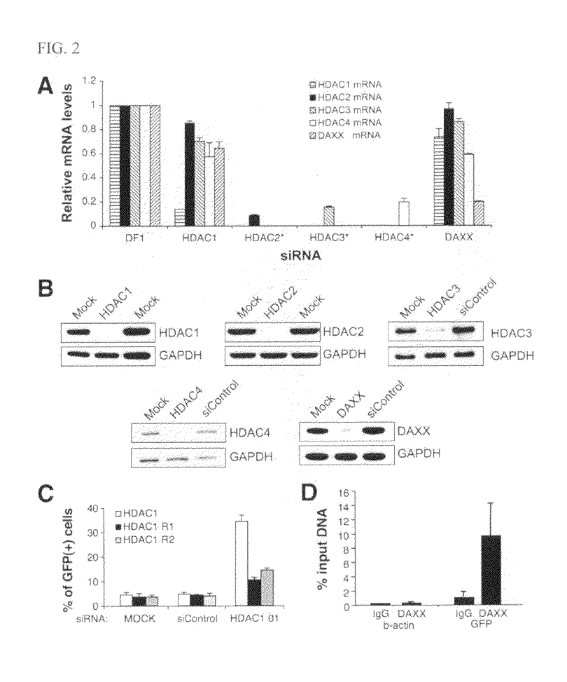

Validation and biological relevance of siRNA-mediated reactivation. To confirm the specificities of the reactivation shown in FIG. 1, target mRNA levels were measured by qRT-PCR (FIG. 2A). The results confirm that treatment with an siRNA pool that targets HDAC1 mRNA resulted in a substantial reduction in HDAC1 mRNA levels but not in Daxx mRNA levels. siRNAs directed against HDAC2, −3, and −4 mRNAs were effective at reducing the levels of their respective target mRNAs. Daxx siRNA treatment resulted in a significant reduction in Daxx mRNA, with only a negligible effect on the HDAC1 mRNA level. Western blotting confirmed that the siRNA pools produced significant reductions in the amounts of the corresponding proteins in all cases (FIG. 2B). Thus, the lack of reactivation by HDAC2, −3, and −4 siRNA pools could not be attributed to ineffectiveness of these siRNAs.

The possibility that “off-target” effects of siRNA pools might lead to GFP reactivation through the knockdown of an unintended mRNA target was also considered. As off-target effects are frequently sequence based, analysis of several single siRNAs was carried out. In these experiments, the TI-C population was treated with each of the four individual siRNAs (25 nM concentration) from the HDAC1 and Daxx pools. Three individual siRNAs from the HDAC1 pool (01, 02, and 09) produced both efficient knockdown of HDAC1 and GFP reactivation. As a final confirmation of specificity, an expression plasmid encoding an siRNA-resistant form of HDAC1 siRNA 01 was constructed, and it was demonstrated that the introduction of this plasmid could repress the GFP reporter in the presence of HDAC1 siRNA 01 (FIG. 2C). All four of the individual Daxx siRNAs produced a level of reactivation that was similar to what was seen for the pool, also eliminating the possibility of off-target effects.

To assess a direct role for Daxx in the maintenance of silencing at the retroviral loci in TI-C cells, chromatin immunoprecipitation (ChIP) was used. As shown in FIG. 2D, Daxx was detected in the hCMV IE promoter/enhanced GFP transcriptional start site region of the silent viral loci but was not detected at the active β-actin cellular locus. Taken together, the siRNA and ChIP results indicate that Daxx plays a direct role in the long-term maintenance of silencing.

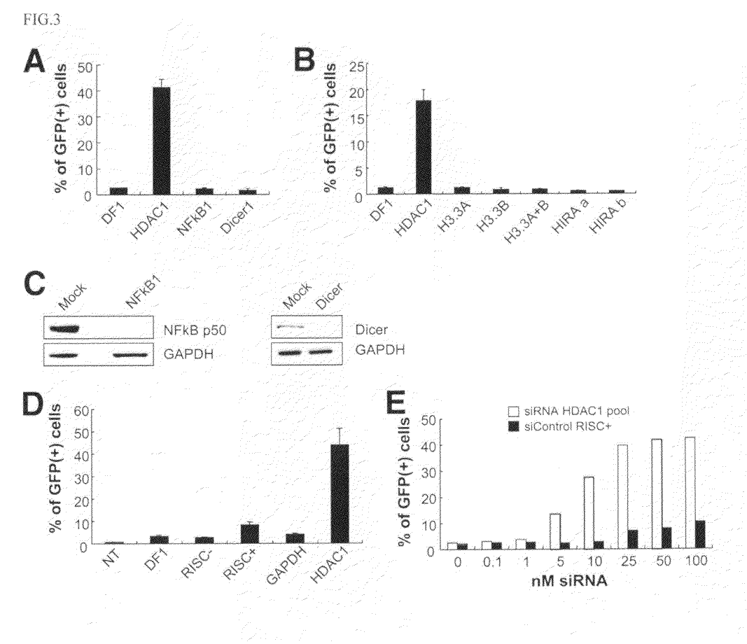

Specific candidate and control siRNAs do not reactivate silent GFP retroviral reporter genes. The effects of other siRNA pools which might have the potential to produce broad effects on HDAC1-mediated silencing (NF-κB), chromatin structure (histone chaperone HIRA and histone H3.3), and microRNA processing (Dicer) were tested. For example, human Dicer1 regulates many genes via its role in microRNA processing and also by production of siRNAs that can direct heterochromatin formation. It was found that the knockdown of NF-κB or Dicer did not result in retroviral reporter gene reactivation (FIGS. 3A and C). The introduction of siRNA pools directed against the histone variants H3.3A and H3.3B or the histone chaperone protein HIRA also failed to reactivate the silent GFP reporter under these conditions (FIG. 3B).

Specifically designed negative control siRNAs (Dharmacon Corp.) that do not target cellular mRNA sequences (nontargeting) but are either able or unable to assemble into the RISC complex that mediates target mRNA degradation were also tested. The nontargeting siRNA can reveal the potential for off-target effects that are mediated through the RISC complex (designated the RISC+ control). The “RISC-free” siRNA is modified to prevent assembly into the RISC complex and serves as a control for the treatment of cells with the transfection reagent in conjunction with a functionally inert RNA payload (designated RISC− control). At 100 nM concentrations, the RISC+ control siRNA showed some low level of reactivation compared to the RISC− or GAPDH siRNA controls (FIG. 3D). As RISC-dependent off-target effects are typically more prominent at high concentrations of individual siRNAs (e.g., 100 nM), this observation gave us an opportunity to more carefully address the effects of siRNA concentration with respect to specific versus off-target effects on GFP reactivation. The standard conditions utilize siRNA pools at a 100 nM final concentration, with each of four siRNAs being present at 25 nM. Parallel titrations of the RISC+ control and the HDAC1 siRNA pool revealed that significant reactivation by the HDAC1 siRNA pool could be observed at concentrations (≧10 nM) where the RISC+ had no effect above what was seen for the transfection reagent alone (FIG. 3E). This titration also revealed that the HDAC1 siRNA pool could reactivate GFP at a low concentration (25 nM), characteristic of specific siRNA targeting. From the experiments shown in FIG. 3, it was concluded that several nonspecific controls, or siRNAs that are predicted to have broad effects on gene expression, do not promote reactivation; furthermore, a role for human Dicer1 in the maintenance of silencing in this system was not identified

In summary, the results of the experiments shown in FIGS. 1 through 3 validated the experimental design, namely, to use siRNAs to interrogate GFP-silent cells to identify factors mediating silencing. These experiments suggest that HDAC1 and Daxx have specific roles in the maintenance of retroviral silencing in this system.

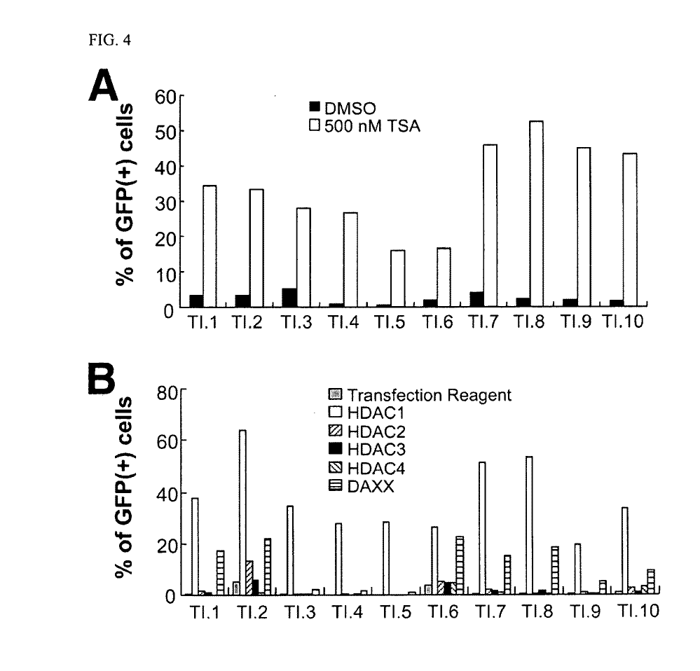

Evidence for position-independent roles for HDAC1 and Daxx in retroviral reporter gene silencing. To determine if the integration sites of the retroviral DNA are determinants for the participation of specific host factors that control epigenetic silencing, a series of TI-C cell clones was examined. These clones were derived from a pool of cells in which the average GFP copy number was ca. 1 as measured by quantitative real-time PCR (Katz et al., 2007, J. Virol. 81:2592-2604; Poleshko et al., 2008, J. Virol. 82:2313-2323). As shown in FIG. 4A, treatment of each of these cell clones with TSA induced GFP reactivation, but to various degrees. Challenge with the HDAC1 siRNA pool also resulted in reactivation in all clones, and the Daxx siRNA pool produced a measurable reactivation in 7 of the 10 clones tested (FIG. 4B, clones 1, 2, 6, 7, 8, 9, and 10). As with the TI-C cell population, transfection with the HDAC1 siRNA pool phenocopied the TSA treatment (compare FIGS. 4A and B). Based on analyses of these clones, it was concluded that HDAC1 and, in most cases, Daxx are required for the maintenance of silencing at independent loci, but the integration site may modulate the extent of reactivation.

Clones 2 and 6 also showed modest levels of reactivation after treatment with HDAC2 and HDAC3 siRNA pools; however, the mean fluorescence intensities of GFP positive cells were significantly lower than that produced by the HDAC1 siRNA pool (not shown). Furthermore, clones 2 and 6 also showed exaggerated responses to the transfection reagent alone, compared to other clones. Clone 2 was also prone to spontaneous or stress-induced GFP reactivation (data not shown). These results also indicate that the integration locus can affect the reactivation properties.

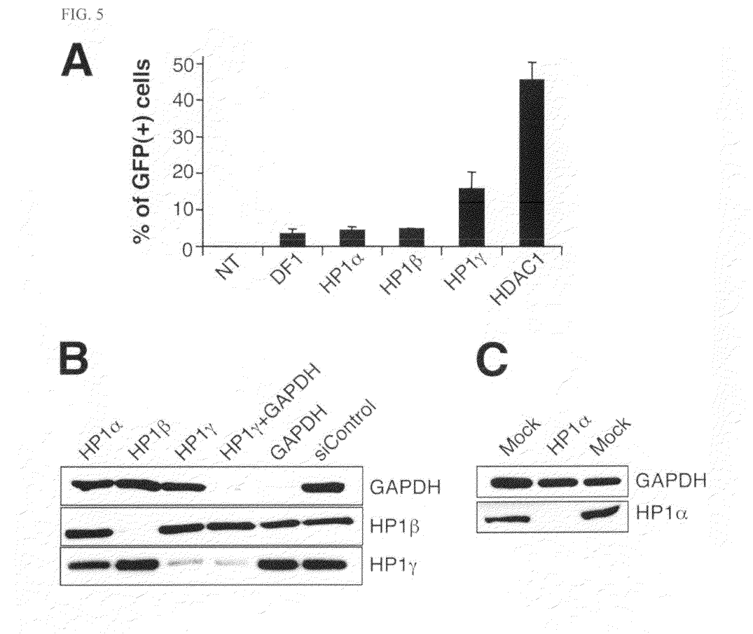

HP1 plays a role in retroviral gene silencing. The three isoforms of HP1, designated α, β, and γ, have been implicated in a variety of processes, including the maintenance of epigenetic gene silencing (Grewal & Jia, 2007, Nat. Rev. Genet. 8:35-46). To investigate the possible role of these proteins in retroviral reporter gene silencing, HeLa TI-C cells were transfected with HP1α, β, and γ siRNA pools. As shown in FIG. 5A, the HP1γ siRNA pool induced significant GFP reactivation, whereas the HP1α and HP1β siRNA pools had no effect compared to what was seen for transfection agent alone. Although transfection of the HP1γ siRNA pool resulted in a smaller percentage of GFP-expressing cells than transfection of the HDAC1 siRNA pool (FIG. 5A), the GFP intensity in the activated cells was high.

The HP1β and HP1γ isoforms were found to be highly abundant in HeLa cells, and siRNA-specific knockdown of both proteins was confirmed (FIG. 5B). The amount of HP1α was very low in untransfected HeLa cells, but knockdown was confirmed using more-sensitive Western blotting conditions (FIG. 5C).

To address the possibility that residual amounts of HP1γ might account for the only limited reactivation produced by the HP1γ siRNA pool, GAPDH and HP1γ siRNA pools, the siRNAs were cotransfected in order to internally monitor the transfection efficiency as measured by GAPDH knockdown. As expected, transfection of the GAPDH siRNA pool resulted in knockdown of GAPDH and had no effect on HP1β and HP1γ levels (FIG. 5B). In GAPDH-HP1γ-cotransfected cells, residual levels of HP1γ could be detected under conditions in which GAPDH was nearly undetectable. It was concluded that either the HP1γ siRNA is pool less potent or a long-lived form of HP1γ protein persists. Thus, the limited reactivation in response to HP1γ siRNAs could be due to the presence of residual HP1γ protein.

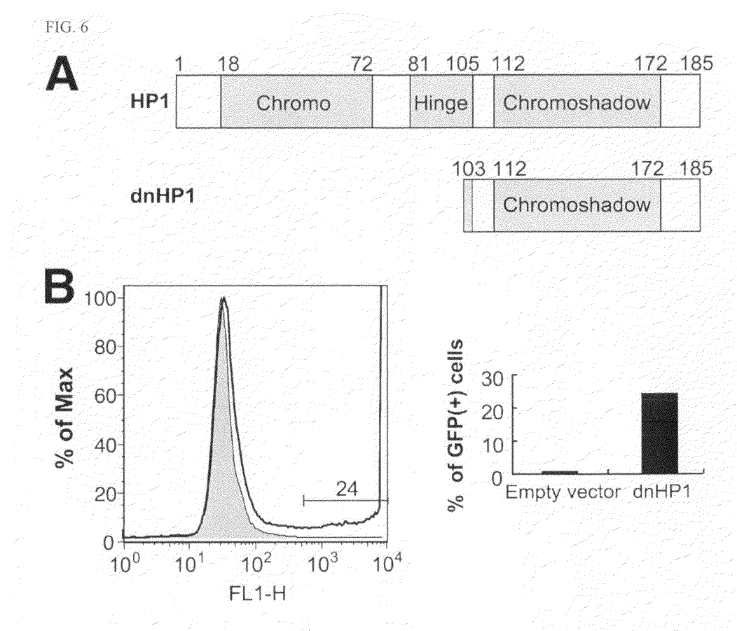

To independently assess a role for HP1 isoforms in silencing, a dominant negative form (dnHP1) was. The dnHP1 was constructed by deleting the chromodomain from HP1β, leaving only the chromoshadow multimerization domain (FIG. 6A). The dnHP1 form was introduced into TI-C cells with a retroviral vector, followed by the selection of transduced cells by use of puromycin. A dramatic reactivation of the GFP gene was observed in a large subset of these cells (6B, right), and the GFP intensity was very high (FIG. 6B, left). Parallel puromycin selection of cells transduced with an empty vector failed to induce GFP expression (FIG. 6B, left). Based on the siRNA results (FIG. 5A), it is likely that the relevant target of inhibition by dnHP1 is HP1γ. It was therefore concluded that HP1γ contributes to the maintenance of retroviral reporter gene silencing in this system.

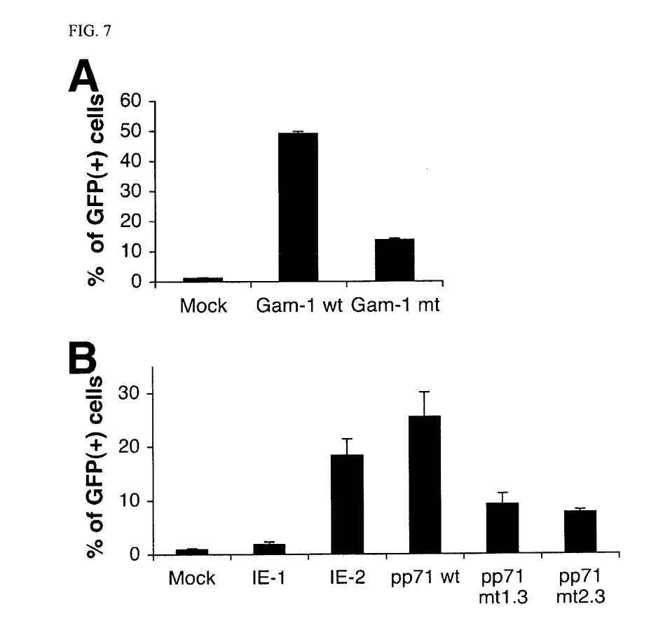

Virus-encoded inhibitors of HDACs and Daxx can reactivate the silent GFP retroviral reporter gene. Several viral proteins are known to bind to and inhibit HDACs. These proteins may act as countermeasures to protect viral genomes from repression by HDACs, consistent with a role for HDACs in an antiviral response. The avian adenovirus protein Gam1 has been demonstrated to inhibit human HDAC1 (Chiocca, et al., 2002, Curr. Biol. 12:594-598), while the hCMV proteins IE1 and IE2 inhibit HDAC3 (Nevels, et al., 2004, Proc. Natl. Acad. Sci. USA 101:17234-17239). As shown in FIG. 7A, transfection of an expression plasmid that encodes the Gam1 protein resulted in a dramatic reactivation of the silent GFP retroviral reporter in the TI-C cell population. A mutant plasmid that encodes a protein with diminished capacity to inhibit HDAC1 showed less of an effect. Expression of the wt and mutant Gam-1 proteins was confirmed by Western blotting. Gam1 was also expressed in TI-C cell clone 3, in which GFP was reactivated only by HDAC1 siRNA (FIG. 4B). Again, expression of Gam1, but not of the Gam1 mutant, resulted in strong retroviral reporter gene reactivation with this clone (data not shown). As Gam1 expression phenocopies the effect of HDAC1 siRNA, this experiment provides independent confirmation that inhibition of HDAC1 is sufficient for reactivation.

Expression of hCMV IE2 but not IE1 resulted in strong retroviral reporter gene reactivation in clone 3 (FIG. 7B). The expression of both proteins was confirmed by Western blotting. Both IE1 and IE2 are reported to form complexes with HDAC3 and inhibit its activity, although broader HDAC inhibitory specificities of these proteins have not been explored. Again, as the siRNA experiments identified a role for HDAC1 but not HDAC3 in silencing maintenance, these findings indicate that IE2 may inhibit HDAC1 as well as HDAC3. Further studies will be required to obtain support for this interpretation. Although Gam1, IE1, and IE2 may have diverse and complex functions beyond HDAC inhibition, the system described herein has apparently provided a means to detect their HDAC-inhibitory activity.

Several studies have identified a role for Daxx in the repression of hCMV gene expression. Furthermore, hCMV encodes a protein, pp71, that inhibits Daxx by targeting it for proteasome-mediated degradation. The fact that hCMV encodes a Daxx “countermeasure” supports a model whereby Daxx mediates an antiviral response that is overcome by pp71. Previous findings suggested a role for Daxx in the initiation of viral transcriptional repression (Greger et al., 2005, J. Virol. 79:4610-4618). The ability of Daxx siRNAs to reactivate silent retroviral DNA in long-term-passage cells implicates a role for Daxx in the maintenance of silencing. As a further test of this interpretation, TI-C cells were transfected with a plasmid encoding hCMV pp71. As shown in FIG. 7B, the transfection of plasmids encoding wt pp71, but not of two mutant forms of this protein, resulted in a robust reactivation of GFP expression, thus providing independent confirmation of a role for Daxx in the maintenance of silencing of integrated retroviral DNA in this system.

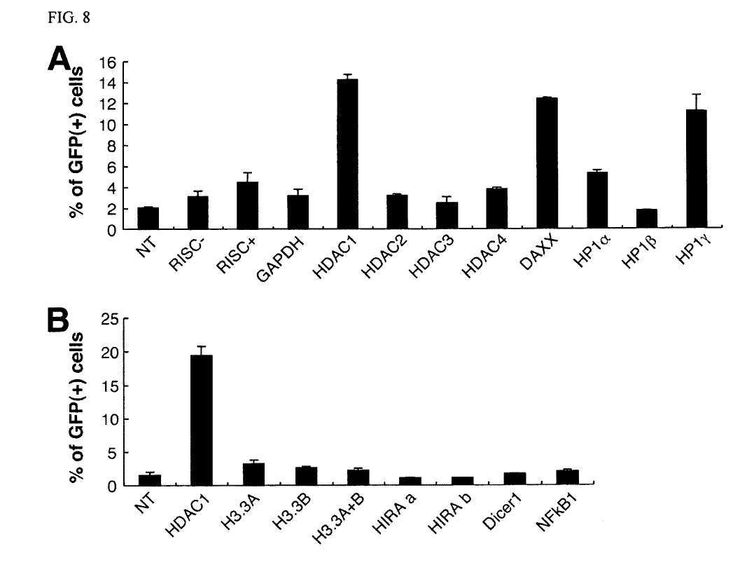

The retroviral reporter gene promoter is not the major determinant of silencing. In the experiments described above, the silent, TSA-sensitive GFP gene was under the control of the hCMV IE promoter (TI-C cells). To determine if the results that were obtained were dependent on this promoter, another cell population was tested in which the silent GFP reporter gene is driven by the native retroviral LTR promoter (TI-L cells) and for which rechallenge with HDIs also results in GFP reactivation (Katz et al., 2007, J. Virol. 81:2592-2604; Poleshko et al., 2008, J. Virol. 82:2313-2323). As shown in FIG. 8, when this cell population was treated with the collection of siRNA pools described above, only HDAC1, Daxx, and HP1γ siRNAs produced reactivation significantly above background levels. The overall detection of the GFP response is reduced compared to what was seen for the TI-C cells, owing to the weaker LTR promoter. Similar results were obtained with a third cell population, in which the silent reporter was under the control of the cellular EF1-α promoter (Katz et al., 2007, J. Virol. 81:2592-2604). From these experiments, it was concluded that the reporter gene promoter is not the major determinant in eliciting the activities of a signature constellation of factors that participate in maintenance of epigenetic silencing.

### Example 2

This example describes the development of a high throughput, siRNA-based screening assay for gene products involved in epigenetic silencing.

A multi-well assay was established in which GFP reactivation could be monitored using a 96-well FACS instrument (Guava). To assess the well-to-well reproducibility of the assay, a Z′-factor was calculated (Zhang, et al., 1999, J. Biomol. Screen 4:67-73), an indicator of the assay quality. In a 96-well plate, alternating rows of cells were treated with the negative control siRNA (GAPDH) and the positive control siRNA, HDAC1 (48 wells each). Analysis of triplicate plates produced a Z′-factor of about 0.8, indicative of a very good assay. A similar assay was also established using a fluorescence plate reader. In this embodiment, a Z′-factor of about 0.6 was obtained, which is well within the range required for high throughput screening (Zhang, et al., 1999, J. Biomol. Screen 4:67-73).

In any siRNA experiment, or siRNA screen, it is important to identify false positive or false negative results. As described in Example 1, these parameters were thoroughly considered and investigated. A false positive is defined as an off-target effect whereby an siRNA knocks down an unintended mRNA target. As off-target effects are siRNA-sequence and siRNA-concentration dependent, siRNA titrations were used and the use of independent siRNAs provide tests for specificity.

Knocking down an intended target may indirectly produce a phenotype by initiating a cascade of cellular events. Such indirect effects may, or may not, be relevant to the biological question being asked. Also, negative results with a particular siRNA could be due to ineffective knockdown of the target, or to induction of cell toxicity which precludes detection of the phenotype being measured. In this assay, it was considered that interference with GFP expression would preclude detection of GFP reactivation. As both false positive and false negative effects are siRNA sequence-dependent, the analysis of multiple siRNAs for each target is important. To address all of these issues, multiple negative control siRNAs were used, and tested two to four independent specific siRNAs for each target. Two assays to detect false negative results were also employed. One assay detects cell toxicity (Alamar blue) produced by specific siRNAs and the other assay detects interference with GFP by measuring a loss of GFP intensity in GFP-expressing cells. Lastly, unless specified, all hits in the screen could be reproduced with two independent cell populations in which the silent GFP was under control of different promoters.

An example protocol is set forth below. This protocol has been performed and tested.

siRNA Resuspension (Day 0)

1. Prepare a 2 μM siRNA masterplate by adding 10 μl of 10 μM siRNA from siRNA stockplate to 40 μl of 1×siRNA suspension buffer. Mix by pipetting carefully up and down.

### Transfection (Day 0)

This protocol has been optimized for use with HeLa cells in a 96-well plate format. The final concentration of siRNA is 50 nM; the final transfection volume is 100 μl; the cell number is 5000 cells/well; the volume of DharmaFECT 1 (Dharmacon, Inc., Boulder Colo.) is 0.15 μl/well.
1. Dilute 30 μl DharmaFECT 1 in 2.97 ml HBSS. Total volume of diluted DharmaFECT 1 is 3.0 ml.
2. Dispense 15.0 μl diluted DharmaFECT 1 into each well of the duplicated reaction plate.
3. Dispense 10.0 μl HBSS into each well of the mixing plate.
4. Add 5.0 μl siRNA from masterplate to the mixing plate. Final volume is 15.0 μl. Mix by pipetting carefully up and down.
5. Dispense 7.0 μl siRNA:HBSS mix to duplicated reaction plate containing DharmaFECT 1. Mix by pipetting carefully up and down. Final volume is 22 μl in each reaction plate. Incubate 20 minutes on RT.
6. While the siRNA and DharmaFECT 1 are complexing, prepare HeLa TI cells in suspension with concentration ˜6000 cells/ml (DMEM, 10% FBS, no drugs).
7. Dispense 80 μl of the HeLa cells suspension to reaction plates containing siRNA:DharmaFECT 1 complex. Final amount of cells is 5000 cells/well.
8. Incubate plates at 37° C. in 5% CO2 for 48 hours.

### End of Transfection (Day 2)

1. Change transfection media to a regular media (DMEM, 10% FBS, pen/strep).

### Measuring Amount of GFP(+) Cells (Day 4)

1. Wash cells with 1× trypsin.
2. Add 60 μl of 1× trypsin and incubate at 37° C. until the cells detach from plate.
3. Add 80 μl of Opti-MEM and mix by pipetting carefully to disrupt cell conglomerates.
4. Measure amount of GFP(+) cells on 96-well Easy-Cite Guava FACS instrument.

### Example 3

This example describes the identification of several modulators of epigenetic silencing using siRNAs directed to 189 mRNA targets, using the assay described in Example 2.

A pre-selected 189 epigenetics siRNA set was designed with targets that include a large collection of chromatin remodeling factors, histone modifying enzymes (HATs, HDACs, histone methyltransferses, histone demethylases) and other epigenetic regulators. The targets are set forth in Table 1. Additional targets are set forth in Table 2.

TABLE 1

| Entrez | | | | Exemplified | |
|---|---|---|---|---|---|
| Gene | | Refseq | | by SEQ ID | |
| ID | Symbol | Transcript | Description | NO(S): | |
| 86 | ACTL6A | NM_178042 | actin-like 6A | | |
| | | NM_177989 | | | |
| | | NM_004301 | | | |
| 546 | ATRX | NM_138270 | alpha | | |
| | | NM_000489 | thalassemia/mental | | |
| | | | retardation syndrome | | |
| | | | X-linked (RAD54 | | |
| | | | homolog, S. cerevisiae) | | |
| 648 | BMI1 | NM_005180 | BMI1 polycomb ring | | |
| | | | finger oncogene | | |
| 1105 | CHD1 | NM_001270 | chromodomain | | |
| | | | helicase DNA | | |
| | | | binding protein 1 | | |
| 1106 | CHD2 | NM_001042572 | chromodomain | | |
| | | NM_001271 | helicase DNA | | |
| | | | binding protein 2 | | |
| 1107 | CHD3 | NM_001005271 | chromodomain | | |
| | | NM_005852 | helicase DNA | | |
| | | NM_001005273 | binding protein 3 | | |
| 1108 | CHD4 | NM_001273 | chromodomain | | |
| | | | helicase DNA | | |
| | | | binding protein 4 | | |
| 1386 | ATF2 | NM_001880 | activating | | |
| | | | transcription factor 2 | | |
| 1387 | CREBBP | NM_001079846 | CREB binding | | |
| | | NM_004380 | protein (Rubinstein- | | |
| | | | Taybi syndrome) | | |
| 1786 | DNMT1 | NM_001379 | DNA (cytosine-5)- | | |
| | | | methyltransferase 1 | | |
| 1787 | TRDMT1 | NM_176083 | tRNA aspartic acid | | |
| | | NM_176081 | methyltransferase 1 | | |
| | | NM_004412 | | | |
| 1788 | DNMT3A | NM_022552 | DNA (cytosine-5-)- | 15 | 16 |
| | | NM_175630 | methyltransferase 3 | | |
| | | NM_153759 | alpha | | |
| | | NM_175629 | | | |
| 1789 | DNMT3B | NM_006892 | DNA (cytosine-5-)- | | |
| | | NM_175850 | methyltransferase 3 | | |
| | | NM_175848 | beta | | |
| | | NM_175849 | | | |
| 1911 | PHC1 | NM_004426 | polyhomeotic | | |
| | | | homolog 1 | | |
| | | | (Drosophila) | | |
| 1912 | PHC2 | NM_004427 | polyhomeotic | 19 | 20 |
| | | NM_198040 | homolog 2 | | |
| | | | (Drosophila) | | |
| 2033 | EP300 | NM_001429 | E1A binding protein | | |
| | | | p300 | | |
| 2074 | ERCC6 | NM_000124 | excision repair cross- | | |
| | | | complementing | | |
| | | | rodent repair | | |
| | | | deficiency, | | |
| | | | complementation | | |
| | | | group 6 | | |
| 2145 | EZH1 | NM_001991 | enhancer of zeste | | |
| | | | homolog 1 | | |
| | | | (Drosophila) | | |
| 2146 | EZH2 | NM_004456 | enhancer of zeste | | |
| | | NM_152998 | homolog 2 | | |
| | | | (Drosophila) | | |
| 2186 | BPTF | NM_004459 | bromodomain PHD | | |
| | | NM_182641 | finger transcription | | |
| | | | factor | | |
| 2648 | GCN5L2 | NM_021078 | GCN5 general control | | |
| | | | of amino-acid | | |
| | | | synthesis 5-like 2 | | |
| | | | (yeast) | | |
| 3065 | HDAC1 | NM_004964 | histone deacetylase 1 | 1 | 2 |
| 3066 | HDAC2 | NM_001527 | histone deacetylase 2 | | |
| 3070 | HELLS | NM_018063 | helicase, lymphoid- | | |
| | | | specific | | |
| 3146 | HMGB1 | NM_002128 | high-mobility group | | |
| | | | box 1 | | |
| 3148 | HMGB2 | NM_002129 | high-mobility group | | |
| | | | box 2 | | |
| 3149 | HMGB3 | NM_005342 | high-mobility group | | |
| | | | box 3 | | |
| 3150 | HMGN1 | NM_004965 | high-mobility group | | |
| | | | nucleosome binding | | |
| | | | domain 1 | | |
| 3151 | HMGN2 | NM_005517 | high-mobility group | | |
| | | | nucleosomal binding | | |
| | | | domain 2 | | |
| 3159 | HMGA1 | NM_145899 | high mobility group | | |
| | | NM_145904 | AT-hook 1 | | |
| | | NM_145901 | | | |
| | | NM_002131 | | | |
| | | NM_145903 | | | |
| | | NM_145902 | | | |
| | | NM_145905 | | | |
| 3276 | PRMT1 | NM_198318 | protein arginine | | |
| | | NM_198319 | methyltransferase 1 | | |
| | | NM_001536 | | | |
| 4152 | MBD1 | NM_015844 | methyl-CpG binding | 7 | 8 |
| | | NM_015847 | domain protein 1 | | |
| | | NM_002384 | | | |
| | | NM_015845 | | | |
| | | NM_015846 | | | |
| 4204 | MECP2 | NM_004992 | methyl CpG binding | | |
| | | | protein 2 (Rett | | |
| | | | syndrome) | | |
| 4261 | CIITA | NM_000246 | class II, major | | |
| | | | histocompatibility | | |
| | | | complex, | | |
| | | | transactivator | | |
| 4297 | MLL | NM_005933 | myeloid/lymphoid or | | |
| | | | mixed-lineage | | |
| | | | leukemia (trithorax | | |
| | | | homolog, Drosophila) | | |
| 4676 | NAP1L4 | NM_005969 | nucleosome assembly | | |
| | | | protein 1-like 4 | | |
| 5885 | RAD21 | NM_006265 | RAD21 homolog | 31 | 32 |
| 5928 | RBBP4 | NM_005610 | retinoblastoma | | |
| | | | binding protein 4 | | |
| 5931 | RBBP7 | NM_002893 | retinoblastoma | | |
| | | | binding protein 7 | | |
| 6015 | RING1 | NM_002931 | ring finger protein 1 | 17 | 18 |
| 6045 | RNF2 | NM_007212 | ring finger protein 2 | | |
| 6046 | BRD2 | NM_005104 | bromodomain | | |
| | | | containing 2 | | |
| 6594 | SMARCA1 | NM_003069 | SWI/SNF related, | | |
| | | | matrix associated, | | |
| | | | actin dependent | | |
| | | | regulator of | | |
| | | | chromatin, subfamily | | |
| | | | a, member 1 | | |
| 6595 | SMARCA2 | NM_139045 | SWI/SNF related, | | |
| | | NM_003070 | matrix associated, | | |
| | | | actin dependent | | |
| | | | regulator of | | |
| | | | chromatin, subfamily | | |
| | | | a, member 2 | | |
| 6596 | HLTF | NM_003071 | helicase-like | | |
| | | NM_139048 | transcription factor | | |
| 6597 | SMARCA4 | NM_003072 | SWI/SNF related, | | |
| | | | matrix associated, | | |
| | | | actin dependent | | |
| | | | regulator of | | |
| | | | chromatin, subfamily | | |
| | | | a, member 4 | | |
| 6598 | SMARCB1 | NM_003073 | SWI/SNF related, | | |
| | | NM_001007468 | matrix associated, | | |
| | | | actin dependent | | |
| | | | regulator of | | |
| | | | chromatin, subfamily | | |
| | | | b, member 1 | | |
| 6599 | SMARCC1 | NM_003074 | SWI/SNF related, | | |
| | | | matrix associated, | | |
| | | | actin dependent | | |
| | | | regulator of | | |
| | | | chromatin, subfamily | | |
| | | | c, member 1 | | |
| 6601 | SMARCC2 | NM_139067 | SWI/SNF related, | | |
| | | NM_003075 | matrix associated, | | |
| | | | actin dependent | | |
| | | | regulator of | | |
| | | | chromatin, subfamily | | |
| | | | c, member 2 | | |
| 6602 | SMARCD1 | NM_003076 | SWI/SNF related, | | |
| | | NM_139071 | matrix associated, | | |
| | | | actin dependent | | |
| | | | regulator of | | |
| | | | chromatin, subfamily | | |
| | | | d, member 1 | | |
| 6603 | SMARCD2 | NM_003077 | SWI/SNF related, | | |
| | | NM_001098426 | matrix associated, | | |
| | | | actin dependent | | |
| | | | regulator of | | |
| | | | chromatin, subfamily | | |
| | | | d, member 2 | | |
| 6749 | SSRP1 | NM_003146 | structure specific | | |
| | | | recognition protein 1 | | |
| 6839 | SUV39H1 | NM_003173 | suppressor of | | |
| | | | variegation 3-9 | | |
| | | | homolog 1 | | |
| | | | (Drosophila) | | |
| 6872 | TAF1 | NM_138923 | TAF1 RNA | | |
| | | NM_004606 | polymerase II, TATA | | |
| | | | box binding protein | | |
| | | | (TBP)-associated | | |
| | | | factor, 250 kDa | | |
| 7290 | HIRA | NM_003325 | HIR histone cell | | |
| | | | cycle regulation | | |
| | | | defective homolog A | | |
| | | | (S. cerevisiae) | | |
| 7703 | PCGF2 | NM_007144 | polycomb group ring | | |
| | | | finger 2 | | |
| 7862 | BRPF1 | NM_001003694 | bromodomain and | | |
| | | NM_004634 | PHD finger | | |
| | | | containing, 1 | | |
| 7994 | MYST3 | NM_006766 | MYST histone | | |
| | | | acetyltransferase | | |
| | | | (monocytic leukemia) 3 | | |
| 8019 | BRD3 | NM_007371 | bromodomain | | |
| | | | containing 3 | | |
| 8085 | MLL2 | NM_003482 | myeloid/lymphoid or | | |
| | | | mixed-lineage | | |
| | | | leukemia 2 | | |
| 8091 | HMGA2 | NM_003483 | high mobility group | | |
| | | NM_003484 | AT-hook 2 | | |
| 8202 | NCOA3 | NM_006534 | nuclear receptor | | |
| | | NM_181659 | coactivator 3 | | |
| 8208 | CHAF1B | NM_005441 | chromatin assembly | | |
| | | | factor 1, subunit B | | |
| | | | (p60) | | |
| 8243 | SMC1A | NM_006306 | structural | | |
| | | | maintenance of | | |
| | | | chromosomes 1A | | |
| 8289 | ARID1A | NM_006015 | AT rich interactive | | |
| | | NM_139135 | domain 1A (SWI- | | |
| | | | like) | | |
| 8438 | RAD54L | NM_003579 | RAD54-like (S. cerevisiae) | | |
| 8458 | TTF2 | NM_003594 | transcription | | |
| | | | termination factor, | | |
| | | | RNA polymerase II | | |
| 8467 | SMARCA5 | NM_003601 | SWI/SNF related, | | |
| | | | matrix associated, | | |
| | | | actin dependent | | |
| | | | regulator of | | |
| | | | chromatin, subfamily | | |
| | | | a, member 5 | | |
| 8520 | HAT1 | NM_003642 | histone | | |
| | | NM_001033085 | acetyltransferase 1 | | |
| 8535 | CBX4 | NM_003655 | chromobox homolog | | |
| | | | 4 (Pc class homolog, | | |
| | | | Drosophila) | | |
| 8648 | NCOA1 | NM_147223 | nuclear receptor | | |
| | | NM_003743 | coactivator 1 | | |
| | | NM_147233 | | | |
| 8726 | EED | NM_152991 | embryonic ectoderm | | |
| | | NM_003797 | development | | |
| 8805 | TRIM24 | NM_003852 | tripartite motif- | 23 | 24 |
| | | NM_015905 | containing 24 | | |
| 8841 | HDAC3 | NM_003883 | histone deacetylase 3 | | |
| 8850 | PCAF | NM_003884 | p300/CBP-associated | | |
| | | | factor | | |
| 8930 | MBD4 | NM_003925 | methyl-CpG binding | | |
| | | | domain protein 4 | | |
| 8932 | MBD2 | NM_015832 | methyl-CpG binding | 9 | 10 |
| | | NM_003927 | domain protein 2 | | |
| 9031 | BAZ1B | NM_032408 | bromodomain | | |
| | | | adjacent to zinc | | |
| | | | finger domain, 1B | | |
| 9044 | BTAF1 | NM_003972 | BTAF1 RNA | | |
| | | | polymerase II, B- | | |
| | | | TFIID transcription | | |
| | | | factor-associated, | | |
| | | | 170 kDa (Mot1 | | |
| | | | homolog, S. cerevisiae) | | |
| 9085 | CDY1 | NM_004680 | chromodomain | | |
| | | NM_170723 | protein, Y-linked, 1 | | |
| 9126 | SMC3 | NM_005445 | structural | | |
| | | | maintenance of | | |
| | | | chromosomes 3 | | |
| 9219 | MTA2 | NM_004739 | metastasis associated | | |
| | | | 1 family, member 2 | | |
| 9324 | HMGN3 | NM_004242 | high mobility group | | |
| | | NM_138730 | nucleosomal binding | | |
| | | | domain 3 | | |
| 9329 | GTF3C4 | NM_012204 | general transcription | | |
| | | | factor IIIC, | | |
| | | | polypeptide 4, 90 kDa | | |
| 9425 | CDYL | NM_004824 | chromodomain | | |
| | | NM_170752 | protein, Y-like | | |
| | | NM_170751 | | | |
| 9426 | CDY2A | NM_004825 | chromodomain | | |
| | | | protein, Y-linked, 2A | | |
| 9557 | CHD1L | NM_004284 | chromodomain | | |
| | | | helicase DNA | | |
| | | | binding protein 1-like | | |
| 9739 | SETD1A | NM_014712 | SET domain | | |
| | | | containing 1A | | |
| 9759 | HDAC4 | NM_006037 | histone deacetylase 4 | | |
| 9867 | PJA2 | NM_014819 | praja 2, RING-H2 | | |
| | | | motif containing | | |
| 9869 | SETDB1 | NM_012432 | SET domain, | 13 | 14 |
| | | NM_001145415 | bifurcated 1 | | |
| 10009 | ZBTB33 | NM_006777 | zinc finger and BTB | | |
| | | | domain containing 33 | | |
| 10013 | HDAC6 | NM_006044 | histone deacetylase 6 | | |
| 10014 | HDAC5 | NM_005474 | histone deacetylase 5 | | |
| | | NM_001015053 | | | |
| 10036 | CHAF1A | NM_005483 | chromatin assembly | 21 | 22 |
| | | | factor 1, subunit A | | |
| | | | (p150) | | |
| 10051 | SMC4 | NM_005496 | structural | | |
| | | NM_001002800 | maintenance of | | |
| | | | chromosomes 4 | | |
| 10155 | TRIM28 | NM_005762 | tripartite motif- | | |
| | | | containing 28 | | |
| 10336 | PCGF3 | NM_006315 | polycomb group ring | | |
| | | | finger 3 | | |
| 10419 | PRMT5 | NM_006109 | protein arginine | | |
| | | NM_001039619 | methyltransferase 5 | | |
| 10473 | HMGN4 | NM_006353 | high mobility group | | |
| | | | nucleosomal binding | | |
| | | | domain 4 | | |
| 10498 | CARM1 | NM_199141 | coactivator-associated | | |
| | | | arginine | | |
| | | | methyltransferase 1 | | |
| 10524 | HTATIP | NM_006388 | HIV-1 Tat interacting | | |
| | | NM_182710 | protein, 60 kDa | | |
| | | NM_182709 | | | |
| 10592 | SMC2 | NM_001042550 | structural | | |
| | | NM_006444 | maintenance of | | |
| | | NM_001042551 | chromosomes 2 | | |
| 10771 | ZMYND11 | NM_212479 | zinc finger, MYND | | |
| | | NM_006624 | domain containing 11 | | |
| 10847 | SRCAP | NM_006662 | Snf2-related CBP | | |
| | | | activator protein | | |
| 10902 | BRD8 | NM_006696 | bromodomain | | |
| | | NM_139199 | containing 8 | | |
| | | NM_183359 | | | |
| 10919 | EHMT2 | NM_025256 | euchromatic histone- | | |
| | | NM_006709 | lysine N- | | |
| | | | methyltransferase 2 | | |
| 10951 | CBX1 | NM_006807 | chromobox homolog | | |
| | | | 1 (HP1 beta homolog | | |
| | | | Drosophila) | | |
| 11143 | MYST2 | NM_007067 | MYST histone | | |
| | | | acetyltransferase 2 | | |
| 11176 | BAZ2A | NM_013449 | bromodomain | | |
| | | | adjacent to zinc | | |
| | | | finger domain, 2A | | |
| 11177 | BAZ1A | NM_182648 | bromodomain | | |
| | | NM_013448 | adjacent to zinc | | |
| | | | finger domain, 1A | | |
| 11335 | CBX3 | NM_007276 | chromobox homolog | | |
| | | NM_016587 | 3 (HP1 gamma | | |
| | | | homolog, Drosophila) | | |
| 22933 | SIRT2 | NM_030593 | sirtuin (silent mating | | |
| | | NM_012237 | type information | | |
| | | | regulation 2 | | |
| | | | homolog) 2 (S. cerevisiae) | | |
| 22955 | SCMH1 | NM_001031694 | sex comb on midleg | | |
| | | NM_012236 | homolog 1 | | |
| | | | (Drosophila) | | |
| 22992 | FBXL11 | NM_012308 | F-box and leucine- | 33 | 34 |
| | | | rich repeat protein 11 | | |
| 23028 | AOF2 | NM_015013 | amine oxidase (flavin | | |
| | | | containing) domain 2 | | |
| 23132 | RAD54L2 | NM_015106 | RAD54-like 2 (S. cerevisiae) | | |
| 23137 | SMC5 | NM_015110 | structural | | |
| | | | maintenance of | | |
| | | | chromosomes 5 | | |
| 23405 | DICER1 | NM_030621 | Dicer1, Dcr-1 | | |
| | | NM_177438 | homolog | | |
| | | | (Drosophila) | | |
| 23411 | SIRT1 | NM_012238 | sirtuin (silent mating | | |
| | | | type information | | |
| | | | regulation 2 | | |
| | | | homolog) 1 (S. cerevisiae) | | |
| 23466 | CBX6 | NM_014292 | chromobox homolog 6 | | |
| 23468 | CBX5 | NM_012117 | chromobox homolog | | |
| | | | 5 (HP1 alpha | | |
| | | | homolog, Drosophila) | | |
| 23476 | BRD4 | NM_058243 | bromodomain | | |
| | | NM_014299 | containing 4 | | |
| 23492 | CBX7 | NM_175709 | chromobox homolog 7 | | |
| 23512 | SUZ12 | NM_015355 | suppressor of zeste 12 | | |
| | | | homolog | | |
| | | | (Drosophila) | | |
| 23522 | MYST4 | NM_012330 | MYST histone | | |
| | | | acetyltransferase | | |
| | | | (monocytic leukemia) 4 | | |
| 23569 | PADI4 | NM_012387 | peptidyl arginine | | |
| | | | deiminase, type IV | | |
| 23613 | ZMYND8 | NM_012408 | zinc finger, MYND- | 37 | 38 |
| | | NM_183047 | type containing 8 | | |
| | | NM_183048 | | | |
| 23774 | BRD1 | NM_014577 | bromodomain | | |
| | | | containing 1 | | |
| 25788 | RAD54B | NM_012415 | RAD54 homolog B | | |
| | | | (S. cerevisiae) | | |
| 25842 | ASF1A | NM_014034 | ASF1 anti-silencing | | |
| | | | function 1 homolog A | | |
| | | | (S. cerevisiae) | | |
| 26038 | CHD5 | NM_015557 | chromodomain | | |
| | | | helicase DNA | | |
| | | | binding protein 5 | | |
| 27127 | SMC1B | NM_148674 | structural | | |
| | | | maintenance of | | |
| | | | chromosomes 1B | | |
| 27443 | CECR2 | NM_031413 | cat eye syndrome | | |
| | | | chromosome region, | | |
| | | | candidate 2 | | |
| 29028 | ATAD2 | NM_014109 | ATPase family, AAA | | |
| | | | domain containing 2 | | |
| 29117 | BRD7 | NM_013263 | bromodomain | | |
| | | | containing 7 | | |
| 29947 | DNMT3L | NM_175867 | DNA (cytosine-5-)- | | |
| | | NM_013369 | methyltransferase 3- | | |
| | | | like | | |
| 29994 | BAZ2B | NM_013450 | bromodomain | | |
| | | | adjacent to zinc | | |
| | | | finger domain, 2B | | |
| 50485 | SMARCAL1 | NM_014140 | SWI/SNF related, | | |
| | | | matrix associated, | | |
| | | | actin dependent | | |
| | | | regulator of | | |
| | | | chromatin, subfamily | | |
| | | | a-like 1 | | |
| 51111 | SUV420H1 | NM_017635 | suppressor of | | |
| | | NM_016028 | variegation 4-20 | | |
| | | | homolog 1 | | |
| | | | (Drosophila) | | |
| 51412 | ACTL6B | NM_016188 | actin-like 6B | | |
| 51564 | HDAC7A | NM_001098416 | histone deacetylase | | |
| | | NM_001098415 | 7A | | |
| | | NM_016596 | | | |
| | | NM_015401 | | | |
| 51592 | TRIM33 | NM_033020 | tripartite motif- | 25 | 26 |
| | | NM_015906 | containing 33 | | |
| 51773 | RSF1 | NM_016578 | remodeling and | | |
| | | | spacing factor 1 | | |
| 53615 | MBD3 | NM_003926 | methyl-CpG binding | 11 | 12 |
| | | | domain protein 3 | | |
| 54014 | BRWD1 | NM_001007246 | bromodomain and | | |
| | | NM_018963 | WD repeat domain | | |
| | | NM_033656 | containing 1 | | |
| 54107 | POLE3 | NM_017443 | polymerase (DNA | | |
| | | | directed), epsilon 3 | | |
| | | | (p17 subunit) | | |
| 54108 | CHRAC1 | NM_017444 | chromatin | | |
| | | | accessibility complex 1 | | |
| 54617 | INOC1 | NM_017553 | INO80 complex | | |
| | | | homolog 1 (S. cerevisiae) | | |
| 54821 | ERCC6L | NM_017669 | excision repair cross- | | |
| | | | complementing | | |
| | | | rodent repair | | |
| | | | deficiency | | |
| | | | complementation | | |
| | | | group 6-like | | |
| 55140 | ELP3 | NM_018091 | elongation protein 3 | | |
| | | | homolog (S. cerevisiae) | | |
| 55193 | PBRM1 | NM_018313 | polybromo 1 | 35 | 36 |
| | | NM_181042 | | | |
| | | NM_018165 | | | |
| 55294 | FBXW7 | NM_018315 | F-box and WD repeat | | |
| | | NM_001013415 | domain containing 7 | | |
| | | NM_033632 | | | |
| 55636 | CHD7 | NM_017780 | chromodomain | | |
| | | | helicase DNA | | |
| | | | binding protein 7 | | |
| 55723 | ASF1B | NM_018154 | ASF1 anti-silencing | | |
| | | | function 1 homolog B | | |
| | | | (S. cerevisiae) | | |
| 55777 | MBD5 | NM_018328 | methyl-CpG binding | | |
| | | | domain protein 5 | | |
| 55869 | HDAC8 | NM_018486 | histone deacetylase 8 | | |
| 55870 | ASH1L | NM_018489 | ash1 (absent, small, | | |
| | | | or homeotic)-like | | |
| | | | (Drosophila) | | |
| 56916 | SMARCAD1 | NM_020159 | SWI/SNF-related, | | |
| | | | matrix-associated | | |
| | | | actin-dependent | | |
| | | | regulator of | | |
| | | | chromatin, subfamily | | |
| | | | a, containing | | |
| | | | DEAD/H box 1 | | |
| 57332 | CBX8 | NM_020649 | chromobox homolog | | |
| | | | 8 (Pc class homolog, | | |
| | | | Drosophila) | | |
| 57634 | EP400 | NM_015409 | E1A binding protein | | |
| | | | p400 | | |
| 57659 | ZBTB4 | NM_020899 | zinc finger and BTB | | |
| | | | domain containing 4 | | |
| 57680 | CHD8 | NM_020920 | chromodomain | | |
| | | | helicase DNA | | |
| | | | binding protein 8 | | |
| 64754 | SMYD3 | NM_022743 | SET and MYND | | |
| | | | domain containing 3 | | |
| 79677 | SMC6 | NM_024624 | structural | | |
| | | | maintenance of | | |
| | | | chromosomes 6 | | |
| 79723 | SUV39H2 | NM_024670 | suppressor of | | |
| | | | variegation 3-9 | | |
| | | | homolog 2 | | |
| | | | (Drosophila) | | |
| 79813 | EHMT1 | NM_024757 | euchromatic histone- | | |
| | | | lysine N- | | |
| | | | methyltransferase 1 | | |
| 79885 | HDAC11 | NM_024827 | histone deacetylase | | |
| | | | 11 | | |
| 80012 | PHC3 | NM_024947 | polyhomeotic | | |
| | | | homolog 3 | | |
| | | | (Drosophila) | | |
| 80854 | SETD7 | NM_030648 | SET domain | | |
| | | | containing (lysine | | |
| | | | methyltransferase) 7 | | |
| 83933 | HDAC10 | NM_032019 | histone deacetylase | | |
| | | | 10 | | |
| 84108 | PCGF6 | NM_032154 | polycomb group ring | | |
| | | NM_001011663 | finger 6 | | |
| 84148 | MYST1 | NM_032188 | MYST histone | | |
| | | | acetyltransferase 1 | | |
| 84181 | CHD6 | NM_032221 | chromodomain | | |
| | | | helicase DNA | | |
| | | | binding protein 6 | | |
| 84333 | PCGF5 | NM_032373 | polycomb group ring | | |
| | | | finger 5 | | |
| 84444 | DOT1L | NM_032482 | DOT1-like, histone | | |
| | | | H3 methyltransferase | | |
| | | | (S. cerevisiae) | | |
| 84678 | FBXL10 | NM_001005366 | F-box and leucine- | | |
| | | NM_032590 | rich repeat protein 10 | | |
| 84733 | CBX2 | NM_005189 | chromobox homolog | | |
| | | NM_032647 | 2 (Pc class homolog, | | |
| | | | Drosophila) | | |
| 84759 | PCGF1 | NM_032673 | polycomb group ring | | |
| | | | finger 1 | | |
| 84787 | SUV420H2 | NM_032701 | suppressor of | 29 | 30 |
| | | | variegation 4-20 | | |
| | | | homolog 2 | | |
| | | | (Drosophila) | | |
| 85509 | MBD3L1 | NM_145208 | methyl-CpG binding | | |
| | | | domain protein 3-like 1 | | |
| 114785 | MBD6 | NM_052897 | methyl-CpG binding | | |
| | | | domain protein 6 | | |
| 124359 | CDYL2 | NM_152342 | chromodomain | | |
| | | | protein, Y-like 2 | | |
| 125997 | MBD3L2 | NM_144614 | methyl-CpG binding | | |
| | | | domain protein 3-like 2 | | |
| 127540 | HMGB4 | NM_145205 | high-mobility group | | |
| | | NM_001008728 | box 4 | | |
| 253175 | CDY1B | NM_001003894 | chromodomain | | |
| | | NM_001003895 | protein, Y-linked, 1B | | |
| 253461 | ZBTB38 | XM_001133510 | zinc finger and BTB | | |
| | | NM_001080412 | domain containing 38 | | |
| 375748 | LOC375748 | NM_001010895 | RAD26L | | |
| | | | hypothetical protein | | |
| 387893 | SETD8 | NM_020382 | SET domain | | |
| | | | containing (lysine | | |
| | | | methyltransferase) 8 | | |
| 1616 | DAXX | NM_001141969 | Homo sapiens death- | 3 | 4 |
| | | NM_001141970 | domain associated | | |
| | | NM_001350 | protein (DAXX) | | |
| 11335 | HP1.gamma. | NM_007276 | HP1 gamma homolog | 5 | 6 |
| | | NM_016587 | | | |
| 9682 | JMJD2 | NM_014663 | KDM4A lysine (K)- | 27 | 28 |
| | | | specific demethylase | | |
| | | | 4A | | |

TABLE 2

| Entrez | | | | Exemplified | |
|---|---|---|---|---|---|
| Gene | | | | by SEQ ID | |
| ID | Symbol | Refseq Transcript | Description | NO(S): | |
| 283208 | P4HA3 | NM_182904 | P4HA3 prolyl 4- | 39 | 40 |
| | | | hydroxylase, alpha | | |
| | | | polypeptide III | | |
| 5783 | PTPN13 | NM_006264 | PTPN13 protein | 41 | 42 |
| | | NM_080683 | tyrosine phosphatase, | | |
| | | NM_080684 | non-receptor type 13 | | |
| | | NM_080685 | (APO-1/CD95 (Fas)- | | |
| | | | associated | | |
| | | | phosphatase) | | |
| 2303 | FOXC2 | NM_005251 | FOXC2 forkhead box | 43 | 44 |
| | | | C2 (MFH-1, | | |
| | | | mesenchyme | | |
| | | | forkhead 1) | | |
| 56954 | NIT2 | NM_020202 | NIT2 nitrilase family, | 45 | 46 |
| | | | member 2 | | |
| 9828 | ARHGEF17 | NM_014786 | ARHGEF17 Rho | 47 | 48 |
| | | | guanine nucleotide | | |
| | | | exchange factor | | |
| | | | (GEF) 17 | | |
| 10861 | SLC26A1 | NM_022042 | SLC26A1 solute | 49 | 50 |
| | | NM_134425 | carrier family 26 | | |
| | | NM_213613 | (sulfate transporter), | | |
| | | | member 1 | | |
| 9313 | MMP20 | NM_004771 | MMP20 matrix | 51 | 52 |
| | | | metallopeptidase 20 | | |
| 1365 | CLDN3 | NM_001306 | CLDN3 claudin 3 | 53 | 54 |
| 10103 | TSPAN-1 | NM_005727 | TSPAN1 tetraspanin 1 | 55 | 56 |
| 3704 | ITPA | NM_033453 | ITPA inosine | 57 | 58 |
| | | NM_181493 | triphosphatase | | |
| | | | (nucleoside | | |
| | | | triphosphate | | |
| | | | pyrophosphatase) | | |
| | LOC400888 | XM_375955 | similar to | 59 | 60 |
| | | | immunoglobulin | | |
| | | | superfamily, member | | |
| | | | 3; immunoglobin | | |
| | | | superfamily, member 3 | | |
| 55196 | FLJ10652 | NM_018169 | C12orf35 | 61 | 62 |
| | | | chromosome 12 open | | |
| | | | reading frame 35 | | |
| 266743 | NXF | NM_178864 | NPAS4 neuronal | 63 | 64 |
| | | | PAS domain protein 4 | | |
| 4502 | MT2A | NM_005953 | MT2A | 65 | 66 |
| | | | metallothionein 2A | | |
| 3083 | HGFAC | NM_001528 | HGFAC HGF | 67 | 68 |
| | | | activator | | |
| 9970 | NR1I3 | NM_001077469 | NR1I3 nuclear | 69 | 70 |
| | | NM_001077470 | receptor subfamily 1, | | |
| | | NM_001077471 | group I, member 3 | | |
| | | NM_001077472 | | | |
| | | NM_001077473 | | | |
| | | NM_001077474 | | | |
| | | NM_001077475 | | | |
| | | NM_001077476 | | | |
| | | NM_001077477 | | | |
| | | NM_001077478 | | | |
| | | NM_001077479 | | | |
| | | NM_001077480 | | | |
| | | NM_001077481 | | | |
| | | NM_001077482 | | | |
| | | NM_005122 | | | |
| 10647 | SCGB1D2 | NM_006551 | SCGB1D2 | 71 | 72 |
| | | | secretoglobin, family | | |
| | | | 1D, member 2 | | |
| 6665 | SOX15 | NM_006942 | SOX15 SRY (sex | 73 | 74 |
| | | | determining region | | |
| | | | Y)-box 15 | | |
| 57408 | HT017 | NM_020678 | LRTM1 leucine-rich | 75 | 76 |
| | | | repeats and | | |
| | | | transmembrane | | |
| | | | domains 1 | | |
| 10982 | MAPRE2 | NM_001143826 | MAPRE2 | 77 | 78 |
| | | NM_001143827 | microtubule- | | |
| | | NM_014268 | associated protein, | | |
| | | | RP/EB family, | | |
| | | | member 2 | | |
| 7329 | UBE2I | NM_003345 | UBE2I ubiquitin- | 79 | 80 |
| | | NM_194259 | conjugating enzyme | | |
| | | NM_194260 | E2I | | |
| | | NM_194261 | | | |
| 55729 | ATF7IP | NM_018179 | ATF7IP activating | 81 | 82 |
| | | | transcription factor 7 | | |
| | | | interacting protein | | |
| 4353 | MPO | NM_000250 | MPO | 83 | 84 |
| | | | myeloperoxidase | | |
| 2618 | GART | NM_000819 | GART | 85 | 86 |
| | | NM_001136005 | phosphoribosylglycin | | |
| | | NM_001136006 | amide | | |
| | | NM_175085 | formyltransferase, | | |
| | | | phosphoribosylglycin | | |
| | | | amide synthetase, | | |
| | | | phosphoribosylamino | | |
| | | | imidazole synthetase | | |
| 9265 | PSCD3 | NM_004227 | CYTH3 cytohesin 3 | 87 | 88 |
| 5927 | JARID1A | NM_001042603 | KDM5A lysine (K)- | 89 | 90 |
| | | NM_005056 | specific demethylase | | |
| | | | 5A | | |
| 4331 | MNAT1 | NM_002431 | MNAT1 menage a | 91 | 92 |
| | | | trois homolog 1, | | |
| | | | cyclin H assembly | | |
| | | | factor | | |
| 5825 | ABCD3 | NM_001122674 | ABCD3 ATP-binding | 93 | 94 |
| | | NM_002858 | cassette, sub-family | | |
| | | | D (ALD), member 3 | | |
| 114825 | KIAA1935 | NM_001130864 | PWWP2A PWWP | 95 | 96 |
| | | NM_052927 | domain containing | | |
| | | | 2A | | |
| 8745 | ADAM23 | NM_003812 | ADAM23 ADAM | 97 | 98 |
| | | | metallopeptidase | | |
| | | | domain 23 | | |
| 283694 | OR4N4 | NM_001005241 | OR4N4 olfactory | 99 | 100 |
| | | | receptor, family 4, | | |
| | | | subfamily N, member 4 | | |
| 7539 | ZFP37 | NM_003408 | ZFP37 zinc finger | 101 | 102 |
| | | | protein 37 homolog | | |
| 2944 | GSTM1 | NM_000561 | GSTM1 glutathione | 103 | 104 |
| | | NM_146421 | S-transferase mu 1 | | |
| 10808 | HSPH1 | NM_006644 | HSPH1 heat shock | 105 | 106 |
| | | | 105 kDa/110 kDa | | |
| | | | protein 1 | | |
| 127670 | HE9 | NM_172000 | TEDDM1 | 107 | 108 |
| | | | transmembrane | | |
| | | | epididymal protein 1 | | |
| 4258 | MGST2 | NM_002413 | MGST2 microsomal | 109 | 110 |
| | | | glutathione S- | | |
| | | | transferase 2 | | |
| 6310 | SCA1 | NM_000332 | ATXN1 ataxin 1 | 111 | 112 |
| | | NM_001128164 | | | |
| 5810 | RAD1 | NM_002853 | RAD1 homolog | 113 | 114 |
| | | NR_026591 | | | |
| 126789 | FLJ90811 | NM_153339 | PUSL1 | 115 | 116 |
| | | | pseudouridylate | | |
| | | | synthase-like 1 | | |
| 23647 | ARFIP2 | NM_012402 | ARFIP2 ADP- | 117 | 118 |
| | | | ribosylation factor | | |
| | | | interacting protein 2 | | |
| 4222 | MEOX1 | NM_001040002 | MEOX1 | 119 | 120 |
| | | NM_004527 | mesenchyme | | |
| | | NM_013999 | homeobox 1 | | |
| 387742 | LOC387742 | NM_001014374 | FAM99A family with | 121 | 122 |
| | | | sequence similarity | | |
| | | | 99, member A | | |
| 3640 | INSL3 | NM_005543 | INSL3 insulin-like 3 | 123 | 124 |
| | | | (Leydig cell) | | |
| 308 | ANXA5 | NM_001154 | annexin A5 | 125 | 126 |
| 6121 | RPE65 | NM_000329 | RPE65 retinal | 127 | 128 |
| | | | pigment epithelium- | | |
| | | | specific protein | | |
| 182 | JAG1 | NM_000214 | Jagged 1 (Alagille | 129 | 130 |
| | | | syndrome) | | |
| 26993 | AKAP8L | NM_014371 | AKAP8L A kinase | 131 | 132 |
| | | | (PRKA) anchor | | |
| | | | protein 8-like | | |
| 10344 | CCL26 | NM_006072 | CCL26 chemokine | 133 | 134 |
| | | | (C-C motif) ligand 26 | | |
| 9651 | PLCL4 | NM_014638 | PLCH2 | 135 | 136 |
| | | | phospholipase C, eta 2 | | |
| 84258 | SYT3 | NM_032298 | SYT3 synaptotagmin | 137 | 138 |
| | | | III | | |
| 91147 | MGC26979 | NM_001142301 | TMEM67 | 139 | 140 |
| | | NM_153704 | transmembrane | | |
| | | | protein 67 | | |
| 727756 | LOC197049 | XM_001125679 | LOC727756 | 141 | 142 |
| 401860 | LOC401860 | XM_377445 | similar to double | 143 | 144 |
| | | | homeobox 4c | | |
| 29850 | TRPM5 | NM_014555 | TRPM5 transient | 145 | 146 |
| | | | receptor potential | | |
| | | | cation channel, | | |
| | | | subfamily M, | | |
| | | | member 5 | | |
| 83740 | H2AFB | NM_080720 | H2A histone family, | 147 | 148 |
| | | | member B3 | | |
| 6009 | RHEB | NM_005614 | Ras homolog | 149 | 150 |
| | | | enriched in brain | | |
| 439938 | FLJ37940 | NM_178534 | LOC439938 | 151 | 152 |
| 10320 | ZNFN1A1 | NM_006060 | IKZF1 IKAROS | 153 | 154 |
| | | | family zinc finger 1 | | |
| | | | (Ikaros) | | |
| 7399 | USH2A | NM_007123 | Usher syndrome 2A | 155 | 156 |
| | | NM_206933 | (autosomal recessive, | | |
| | | | mild) | | |
| 9915 | ARNT2 | NM_014862 | Aryl-hydrocarbon | 157 | 158 |
| | | | receptor nuclear | | |
| | | | translocator 2 | | |
| 55080 | TAPBPL | NM_018009 | TAP binding protein- | 159 | 160 |
| | | | like | | |
| 5774 | PTPN3 | NM_001145368 | Protein tyrosine | 161 | 162 |
| | | NM_001145369 | phosphatase, non- | | |
| | | NM_001145370 | receptor type 3 | | |
| | | NM_001145371 | | | |
| | | NM_001145372 | | | |
| | | NM_002829 | | | |
| 608 | TNFRSF17 | NM_001192 | Tumor necrosis factor | 163 | 164 |
| | | | receptor superfamily, | | |
| | | | member 17 | | |
| 10054 | UBA2 | NM_005499 | Ubiquitin-like | 165 | 166 |
| | | | modifier activating | | |
| | | | enzyme 2 | | |
| 83872 | FIBL-6 | NM_031935 | HMCN1 hemicentin 1 | 167 | 168 |
| 23469 | PHF3 | NM_015153 | PHD finger protein 3 | 169 | 170 |
| 66004 | LYNX1 | NM_023946 | Ly6/neurotoxin 1 | 171 | 172 |
| | | NM_177457 | | | |
| | | NM_177458 | | | |
| | | NM_177476 | | | |
| | | NM_177477 | | | |
| 79893 | ZNF403 | NM_024835 | GGNBP2 | 173 | 174 |
| | | | gametogenetin | | |
| | | | binding protein 2 | | |
| 5009 | OTC | NM_000531 | Ornithine | 175 | 176 |
| | | | carbamoyltransferase | | |
| | LOC56270 | CR456770 | Homo sapiens full | 177 | 178 |
| | | | open reading frame | | |
| | | | cDNA clone | | |
| | | | RZPDo834E014D for | | |
| | | | gene LOC56270, | | |
| | | | hypothetical protein | | |
| | | | 628 | | |
| 5051 | PAFAH2 | NM_000437 | Platelet-activating | 179 | 180 |
| | | | factor | | |
| | | | acetylhydrolase 2 | | |
| 282973 | C10ORF39 | NM_001105521 | JAKMIP3 janus | 181 | 182 |
| | | | kinase and | | |
| | | | microtubule | | |
| | | | interacting protein 3 | | |
| 159119 | HSFY2 | NM_001001877 | Heat shock | 183 | 184 |
| | | NM_153716 | transcription factor, | | |
| | | | Y linked 2 | | |
| 604 | BCL6 | NM_001130845 | B-cell | 185 | 186 |
| | | NM_001134738 | CLL/lymphoma 6 | | |
| | | NM_001706 | | | |
| 57026 | PDXP | NM_020315 | Pyridoxal | 187 | 188 |
| | | | (pyridoxine, vitamin | | |
| | | | B6) phosphatase | | |
| 7044 | EBAF | NM_003240 | LEFTY2 left-right | 189 | 190 |
| | | | determination factor 2 | | |
| 902 | CCNH | NM_001239 | Cyclin H | 191 | 192 |
| | LOC388726 | XM_371335 | Homo sapiens similar | 193 | 194 |
| | | | to osteotesticular | | |
| | | | protein tyrosine | | |
| | | | phosphatase | | |
| 5624 | PC | NM_000312 | PROC protein C | 195 | 196 |
| | | | (inactivator of | | |
| | | | coagulation factors | | |
| | | | Va and VIIIa) | | |
| 4857 | NOVA1 | NM_002515 | Neuro-oncological | 197 | 198 |
| | | NM_006489 | ventral antigen 1 | | |
| | | NM_006491 | | | |
| 4277 | MICB | NM_005931 | MHC class I | 199 | 200 |
| | | | polypeptide-related | | |
| | | | sequence B | | |
| 6923 | TCEB2 | NM_007108 | Transcription | 201 | 202 |
| | | NM_207013 | elongation factor B | | |
| | | | (SIII), polypeptide 2 | | |
| | | | (18 kDa, elongin B) | | |
| 91801 | LOC91801 | NM_138775 | ALKBH8 alkB, | 203 | 204 |
| | | | alkylation repair | | |
| | | | homolog 8 | | |
| 1047 | CLGN | NM_001130675 | Calmegin | 205 | 206 |
| | | NM_004362 | | | |
| 3638 | INSIG1 | NM_005542 | Insulin induced gene 1 | 207 | 208 |
| | | NM_198336 | | | |
| | | NM_198337 | | | |
| 3790 | KCNS3 | NM_002252 | Potassium voltage- | 209 | 210 |
| | | | gated channel, | | |
| | | | delayed-rectifier, | | |
| | | | subfamily S, member 3 | | |
| 1979 | EIF4EBP2 | NM_004096 | Eukaryotic | 211 | 212 |
| | | | translation initiation | | |
| | | | factor 4E binding | | |
| | | | protein 2 | | |
| 136259 | KLF14 | NM_138693 | Kruppel-like factor | 213 | 214 |
| | | | 14 | | |
| 284904 | SEC14L4 | NM_174977 | SEC14-like 4 | 215 | 216 |
| 388512 | FLJ45910 | NM_207390 | CLEC17A C-type | 217 | 218 |
| | | | lectin domain family | | |
| | | | 17, member A | | |
| 55867 | SLC22A11 | NM_018484 | Solute carrier family | 219 | 220 |
| | | | 22 (organic | | |
| | | | anion/urate | | |
| | | | transporter), member | | |
| | | | 11 | | |
| 3655 | ITGA6 | NM_000210 | Integrin, alpha 6 | 221 | 222 |
| | | NM_001079818 | | | |
| 388467 | LOC388467 | XM_373775.1 | LOC388467 | 223 | 224 |
| | | | hypothetical | | |
| 25855 | BRMS1 | NM_001024957 | Breast cancer | 225 | 226 |
| | | NM_015399 | metastasis suppressor 1 | | |
| 919 | CD3Z | NM_000734 | CD247 molecule | 227 | 228 |
| | | NM_198053 | | | |
| 2220 | FCN2 | NM_004108 | Ficolin | 229 | 230 |
| | | NM_015837 | (collagen/fibrinogen | | |
| | | | domain containing | | |
| | | | lectin) 2 (hucolin) | | |
| 7511 | XPNPEP1 | NM_020383 | X-prolyl | 231 | 232 |
| | | | aminopeptidase | | |
| | | | (aminopeptidase P) 1, | | |
| | | | soluble | | |
| 84103 | DKFZP434G072 | NM_032149 | Chromosome 4 open | 233 | 234 |
| | | | reading frame 17 | | |
| 27302 | BMP10 | NM_014482 | Bone morphogenetic | 235 | 236 |
| | | | protein 10 | | |
| 9256 | BZRAP1 | NM_004758 | Benzodiazapine | 237 | 238 |
| | | NM_024418 | receptor (peripheral) | | |
| | | | associated protein 1 | | |
| 84836 | MGC15429 | NM_001146314 | Abhydrolase domain | 239 | 240 |
| | | NM_032750 | containing 14B | | |
| 729 | C6 | NM_000065 | Complement | 241 | 242 |
| | | NM_001115131 | component 6 | | |
| 503694 | DEFB108 | XM_001720423 | Defensin, beta 108, | 243 | 244 |
| | | | pseudogene 1 | | |
| 400948 | LOC400948 | XM_376043 | Similar to CG33774- | 245 | 246 |
| | | | PA | | |
| 255027 | FLJ39599 | NM_001128423 | MPV17 | 247 | 248 |
| | | NM_173803 | mitochondrial | | |
| | | | membrane protein- | | |
| | | | like | | |
| 29058 | C20ORF30 | NM_001009923 | C20ORF30 | 249 | 250 |
| | | NM_001009924 | | | |
| 405 | ARNT | NM_001668 | Aryl hydrocarbon | 251 | 252 |
| | | NM_178426 | receptor nuclear | | |
| | | NM_178427 | translocator | | |
| 196051 | PPAPDC1 | NM_001030059 | Phosphatidic acid | 253 | 254 |
| | | | phosphatase type 2 | | |
| | | | domain containing | | |
| | | | 1A | | |
| 8742 | TNFSF12 | NM_003809 | Tumor necrosis factor | 255 | 256 |
| | | | (ligand) superfamily, | | |
| | | | member 12 | | |
| 6385 | SDC4 | NM_002999 | Syndecan 4 | 257 | 258 |
| 54766 | BTG4 | NM_017589 | B-cell translocation | 259 | 260 |
| | | | gene 4 | | |
| 23157 | SEPT6 | NM_015129 | Septin 6 | 261 | 262 |
| | | NM_145799 | | | |
| | | NM_145800 | | | |
| | | NM_145802 | | | |
| 27177 | IL1F8 | NM_014438 | interleukin 1 family, | 263 | 264 |
| | | | member 8 | | |
| 3586 | IL10 | NM_000572 | interleukin 10 | 265 | 266 |
| 9381 | OTOF | NM_194248 | otoferlin | 267 | 268 |
| 10121 | ACTR1A | NM_005736 | actin-related protein 1 | 269 | 270 |
| | | | homolog A | | |
| 7620 | ZNF69 | NM_021915 | zinc finger protein 69 | 271 | 272 |
| 55605 | KIF21A | NM_017641 | kinesin family | 273 | 274 |
| | | | member 21A | | |
| 3587 | IL10RA | NM_001558 | interleukin 10 | 275 | 276 |
| | | | receptor, alpha | | |
| 2108 | ETFA | NM_000126 | electron-transfer- | 277 | 278 |
| | | NM_001127716 | flavoprotein, alpha | | |
| | | | polypeptide | | |
| 27430 | MAT2B | NM_013283 | methionine | 279 | 280 |
| | | NM_1827 | adenosyltransferase II | | |
| 78994 | MGC3121 | NM_024031 | proline rich 14 (aka | 281 | 282 |
| | | | MGC3121) | | |
| 51719 | MO25 | NM_001130849 | Aka calcium binding | 283 | 284 |
| | | NM_001130850 | protein 39 | | |
| | | NM_016289 | | | |
| 8412 | BCAR3 | NM_003567 | breast cancer anti- | 285 | 286 |
| | | | estrogen resistance 3 | | |
| 402 | ARL2 | NM_001667 | ADP-ribosylation | 287 | 288 |
| | | | factor-like 2 | | |
| 79155 | TNIP2 | NM_024309 | TNFAIP3 interacting | 289 | 290 |
| | | | protein 2 | | |
| 904 | CCNT1 | NM_001240 | cyclin T1 | 291 | 292 |
| 3560 | IL2RB | NM_000878 | interleukin 2 | 293 | 294 |
| | | | receptor, beta | | |
| 8655 | DNCL1 | NM_003746 | Aka: dynein, light | 295 | 296 |
| | | NM_001037495 | chain, LC8-type 1 | | |
| | | NM_001037494 | | | |
| 3681 | ITGAD | NM_005353 | integrin, alpha D | 297 | 298 |
| 9564 | BCAR1 | NM_014567 | breast cancer anti- | 299 | 300 |
| | | | estrogen resistance 1 | | |
| 3675 | ITGA3 | NM_005501 | integrin, alpha 3 | 301 | 302 |
| | | NM_002204 | (antigen CD49C, | | |
| | | | alpha 3 subunit of | | |
| | | | VLA-3 receptor) | | |
| 53938 | PPIL3 | NM_131916 | peptidylprolyl | 303 | 304 |
| | | NM_032472 | isomerase | | |
| | | NM_130906 | (cyclophilin)-like 3 | | |
| 55632 | KIAA1333 | NM_017769 | AKA: G2/M-phase | 305 | 306 |
| | | | specific E3 ubiquitin | | |
| | | | ligase | | |
| 1175 | AP2S1 | NM_004069 | adaptor-related | 307 | 308 |
| | | NM_021575 | protein complex 2, | | |
| | | | sigma 1 subunit | | |
| 10252 | SPRY1 | NM_005841 | sprouty homolog 1, | 309 | 310 |
| | | NM_199327 | antagonist of FGF | | |
| | | | signaling | | |
| | | | (Drosophila) | | |
| 64344 | HIF3A | NM_022462 | hypoxia inducible | 311 | 312 |
| | | NM_152794 | factor 3, alpha | | |
| | | NM_152795 | subunit | | |
| 53371 | NUP54 | NM_017426 | nucleoporin 54 kDa | 313 | 314 |
| 23765 | IL17R | NM_014339 | AKA: interleukin 17 | 315 | 316 |
| | | | receptor A | | |
| 79843 | FLJ22746 | NM_001122779 | AKA: family with | 317 | 318 |
| | | NM_024785 | sequence similarity | | |
| | | | 124B | | |
| 2701 | GJA4 | NM_002060 | gap junction protein, | 319 | 320 |
| | | | alpha 4, 37 kDa | | |
| 6773 | STAT2 | NM_005419 | signal transducer and | 321 | 322 |
| | | | activator of | | |
| | | | transcription 2 | | |
| 7469 | WHSC2 | NM_005663 | Wolf-Hirschhorn | 323 | 324 |
| | | | syndrome candidate 2 | | |
| 23456 | ABCB10 | NM_012089 | ATP-binding | 325 | 326 |
| | | | cassette, sub-family | | |
| | | | B (MDR/TAP), | | |
| | | | member 10 | | |
| 54921 | DERPC | NM_001039690 | Aka: chromosome | 327 | 328 |
| | | NM_001040144 | transmission fidelity | | |
| | | NM_001040146 | factor 8 homolog | | |
| 9053 | MAP7 | NM_003980 | microtubule- | 329 | 330 |
| | | | associated protein 7 | | |
| 689 | BTF3 | NM_001037637 | basic transcription | 331 | 332 |
| | | NM_001207 | factor 3 | | |
| 25766 | HYPC | NM_001031698 | PRP40 pre-mRNA | 333 | 334 |
| | | NM_012272 | processing factor 40 | | |
| | | | homolog B | | |
| 4607 | MYBPC3 | NM_000256 | myosin binding | 335 | 336 |
| | | | protein C | | |
| 53373 | TPCN1 | NM_001143819 | two pore segment | 337 | 338 |
| | | NM_017901 | channel 1 | | |
| 27287 | VENTX2 | NM_014468 | Aka: VENT | 339 | 340 |
| | | | homeobox homolog | | |
| | | | (Xenopus laevis) | | |
| 114795 | KIAA1786 | NM_052907 | Aka: TMEM132B | 341 | 342 |
| | | | transmembrane | | |
| | | | protein 132B | | |
| 64077 | LHPP | NM_022126 | phospholysine | 343 | 344 |
| | | | phosphohistidine | | |
| | | | inorganic | | |
| | | | pyrophosphate | | |
| | | | phosphatase | | |
| 439935 | MGC24125 | XR_000543 | hypothetical protein | 345 | 346 |
| | | XR_000648 | MGC24125 | | |
| 6018 | RLF | NM_012421 | rearranged L-myc | 347 | 348 |
| | | | fusion | | |
| 4507 | MTAP | NM_002451 | methylthioadenosine | 349 | 350 |
| | | | phosphorylase | | |
| 81488 | GRINL1A | NM_001018102 | glutamate receptor, | 351 | 352 |
| | | NM_015532 | ionotropic, N-methyl | | |
| | | | D-aspartate-like 1A | | |
| 7298 | TYMS | NM_001071 | thymidylate | 353 | 354 |
| | | | synthetase | | |
| 132160 | FLJ32332 | NM_001122870 | Aka: protein | 355 | 356 |
| | | NM_144641 | phosphatase 1M | | |
| | | | (PP2C domain | | |
| | | | containing) | | |
| 125919 | ZNF543 | NM_213598 | zinc finger protein | 357 | 358 |
| | | | 543 | | |
| 3664 | IRF6 | NM_006147 | interferon regulatory | 359 | 360 |
| | | | factor 6 | | |
| 400475 | LOC400475 | XM_378553.2 | hypothetical | 361 | 362 |
| | | | LOC400475 | | |
| 285676 | ZNF454 | NM_182594 | zinc finger protein | 363 | 364 |
| | | | 454 | | |
| 338872 | MGC48915 | NM_178540 | AKA: C1q and tumor | 365 | 366 |
| | | | necrosis factor related | | |
| | | | protein 9 | | |
| 9708 | PCDHGA8 | NM_014004 | protocadherin gamma | 367 | 368 |
| | | NM_032088 | subfamily A, 8 | | |
| 146325 | LOC146325 | NM_145270 | C16orf11 | 369 | 370 |
| | | | chromosome 16 open | | |
| | | | reading frame 11 | | |
| 10806 | SDCCAG8 | NM_006642 | serologically defined | 371 | 372 |
| | | | colon cancer antigen 8 | | |
| 140767 | VMP | NM_080723 | AKA: neurensin 1 | 373 | 374 |
| 10801 | MSF | NM_001113491 | Akia: SEPT9 septin 9 | 375 | 376 |
| | | NM_001113492 | | | |
| | | NM_001113493 | | | |
| | | NM_001113494 | | | |
| | | NM_001113495 | | | |
| | | NM_001113496 | | | |
| | | NM_006640 | | | |
| 83729 | INHBE | NM_031479 | inhibin, beta E | 377 | 378 |
| 11278 | KLF12 | NM_007249 | Kruppel-like factor | 379 | 380 |
| | | | 12 | | |
| 403314 | MGC26594 | NM_203454 | AKA: apolipoprotein | 381 | 382 |
| | | | B mRNA editing | | |
| | | | enzyme, catalytic | | |
| | | | polypeptide-like 4 | | |
| | | | (putative) | | |
| 285367 | MGC29784 | NM_001142547 | AKA: RNA | 383 | 384 |
| | | NM_173659 | pseudouridylate | | |
| | | | synthase domain | | |
| | | | containing 3 | | |
| 83463 | MXD3 | NM_001142935 | MAX dimerization | 385 | 386 |
| | | NM_031300 | protein 3 | | |
| 53981 | CPSF2 | NM_017437 | cleavage and | 387 | 388 |
| | | | polyadenylation | | |
| | | | specific factor 2, | | |
| | | | 100 kDa | | |
| 5981 | RFC1 | NM_002913 | replication factor C | 389 | 390 |
| | | | (activator 1) 1, | | |
| | | | 145 kDa | | |
| 322 | APBB1 | NM_001164 | amyloid beta (A4) | 391 | 392 |
| | | NM_145689 | precursor protein- | | |
| | | | binding, family B, | | |
| | | | member 1 (Fe65) | | |
| 126353 | C19ORF21 | NM_173481 | chromosome 19 open | 393 | 394 |
| | | | reading frame 21 | | |
| 9771 | RAPGEF5 | NM_012294 | Rap guanine | 395 | 396 |
| | | | nucleotide exchange | | |
| | | | factor (GEF) 5 | | |
| 10398 | MYL9 | NM_006097 | myosin, light chain 9, | 397 | 398 |
| | | NM_181526 | regulatory | | |
| 10846 | PDE10A | NM_001130690 | phosphodiesterase | 399 | 400 |
| | | NM_006661 | 10A | | |
| 26509 | FER1L3 | NM_013451 | AKA: myoferlin | 401 | 402 |
| | | NM_133337 | | | |
| 4301 | MLLT4 | NM_001040000 | myeloid/lymphoid or | 403 | 404 |
| | | NM_001040001 | mixed-lineage | | |
| | | NM_005936 | leukemia (trithorax | | |
| | | | homolog, | | |
| | | | Drosophila); | | |
| | | | translocated to, 4 | | |
| 10055 | SAE1 | NM_001145713 | SUMO1 activating | 405 | 406 |
| | | NM_001145714 | enzyme subunit 1 | | |
| | | NM_005500 | | | |
| 5008 | OSM | NM_020530 | oncostatin M | 407 | 408 |
| 166647 | GPR125 | NM_145290 | G protein-coupled | 409 | 410 |
| | | | receptor 125 | | |
| 84720 | PIGO | NM_032634 | phosphatidylinositol | 411 | 412 |
| | | NM_152850 | glycan anchor | | |
| | | | biosynthesis, class O | | |
| 11318 | ADMR | NM_007264 | Aka: G protein- | 413 | 414 |
| | | | coupled receptor 182 | | |
| 203197 | C9ORF91 | NM_153045 | chromosome 9 open | 415 | 416 |
| | | | reading frame 91 | | |
| 390079 | LOC390079 | NM_001005168 | OR52E8 olfactory | 417 | 418 |
| | | | receptor, family 52, | | |
| | | | subfamily E, member | | |
| 11272 | PRR4 | NM_001098538 | proline rich 4 | 419 | 420 |
| | | NM_007244 | (lacrimal) | | |
| 390031 | LOC390031 | XM_372343 | SSU72 RNA | 421 | 422 |
| | | XM_939626 | polymerase II CTD | | |
| | | XM_001718915 | phosphatase homolog | | |
| | | | pseudogene | | |
| 4504 | MT3 | NM_005954 | metallothionein 3 | 423 | 424 |
| 26747 | NUFIP1 | NM_012345 | nuclear fragile X | 425 | 426 |
| | | | mental retardation | | |
| | | | protein interacting | | |
| | | | protein 1 | | |
| 79088 | ZNF426 | NM_024106 | zinc finger protein | 427 | 428 |
| | | | 426 | | |
| 6329 | SCN4A | NM_000334 | sodium channel, | 429 | 430 |
| | | | voltage-gated, type | | |
| | | | IV, alpha subunit | | |
| 200197 | FLJ23703 | NM_182534 | chromosome 1 open | 431 | 432 |
| | | | reading frame 126 | | |
| 2001 | ELF5 | NM_001422 | E74-like factor 5 (ets | 433 | 434 |
| | | NM_198381 | domain transcription | | |
| | | | factor) | | |
| 540 | ATP7B | NM_000053 | ATPase, Cu++ | 435 | 436 |
| | | NM_001005918 | transporting, beta | | |
| | | | polypeptide | | |
| 93081 | LOC93081 | NM_138779 | Chromosome 13 open | 437 | 438 |
| | | | reading frame 27 | | |
| 114803 | KIAA1915 | NM_001085487 | Myb-like, SWIRM | 439 | 440 |
| | | | and MPN domains 1 | | |
| 5407 | PNLIPRP1 | NM_006229 | Pancreatic lipase- | 441 | 442 |
| | | | related protein 1 | | |
| 26063 | DECR2 | NM_020664 | 2,4-dienoyl CoA | 443 | 444 |
| | | | reductase 2, | | |
| | | | peroxisomal | | |
| 26261 | FBXO24 | NM_012172 | F-box protein 24 | 445 | 446 |
| | | NM_033506 | | | |
| 84450 | ZNF512 | NM_032434 | Zinc finger protein | 447 | 448 |
| | | | 512 | | |
| 93624 | MGC21874 | NM_152293 | Transcriptional | 449 | 450 |
| | | | adaptor 2 (ADA2 | | |
| | | | homolog, yeast)-beta | | |
| 5817 | PVR | NM_001135768 | Poliovirus receptor | 451 | 452 |
| | | NM_001135769 | | | |
| | | NM_001135770 | | | |
| | | NM_006505 | | | |
| 84268 | MGC4189 | NM_001033002 | RPA interacting | 453 | 454 |
| | | | protein | | |
| 120227 | CYP2R1 | NM_024514 | Cytochrome P450, | 455 | 456 |
| | | | family 2, subfamily | | |
| | | | R, polypeptide 1 | | |
| 7453 | WARS | NM_004184 | Tryptophanyl-tRNA | 457 | 458 |
| | | NM_173701 | synthetase | | |
| | | NM_213645 | | | |
| | | NM_213646 | | | |
| 83990 | BRIP1 | NM_032043 | BRCA1 interacting | 459 | 460 |
| | | | protein C-terminal | | |
| | | | helicase 1 | | |
| 146310 | RNF151 | NM_174903 | Ring finger protein | 461 | 462 |
| | | | 151 | | |
| 124152 | MGC35048 | NM_153208 | IQ motif containing K | 463 | 464 |
| 135114 | HINT3 | NM_138571 | Histidine triad | 465 | 466 |
| | | | nucleotide binding | | |
| | | | protein 3 | | |
| 143686 | SESN3 | NM_144665 | Sestrin 3 | 467 | 468 |
| 157310 | MGC22776 | NM_144962 | Phosphatidylethanolamine- | 469 | 470 |
| | | | binding protein 4 | | |
| 7187 | TRAF3 | NM_003300 | TNF receptor- | 471 | 472 |
| | | NM_145725 | associated factor 3 | | |
| | | NM_145726 | | | |
| 26548 | ITGB1BP2 | NM_012278 | Integrin beta 1 | 473 | 474 |
| | | | binding protein | | |
| | | | (melusin) 2 | | |
| 1271 | CNTFR | NM_001842 | Ciliary neurotrophic | 475 | 476 |
| | | NM_147164 | factor receptor | | |
| 84081 | DKFZP434K1421 | NM_032141 | Coiled-coil domain | 477 | 478 |
| | | | containing 55 | | |
| 150684 | COMMD1 | NM_152516 | Copper metabolism | 479 | 480 |
| | | | (Murr1) domain | | |
| | | | containing 1 | | |
| 151325 | MYADML | NM_207329 | myeloid-associated | 481 | 482 |
| | | | differentiation | | |
| | | | marker-like | | |
| 2893 | GRIA4 | NM_000829 | glutamate receptor, | 483 | 484 |
| | | NM_001077243 | ionotrophic, AMPA 4 | | |
| | | NM_001077244 | | | |
| | | NM_001112812 | | | |
| 412 | STS | NM_000351 | steroid sulfatase | 485 | 486 |
| | | | (microsomal), | | |
| | | | isozyme S | | |
| 83787 | SVH | NM_031905 | Aka: armadillo repeat | 487 | 488 |
| | | | containing 10 | | |
| 3381 | IBSP | NM_004967 | integrin-binding | 489 | 490 |
| | | | sialoprotein | | |
| 91408 | MGC23908 | NM_001136497 | basic transcription | 491 | 492 |
| | | NM_152265 | factor 3-like 4 | | |
| 386680 | KRTAP18-5 | NM_198694 | KRTAP10-5 keratin | 493 | 494 |
| | | | associated protein 10-5 | | |
| 84687 | PPP1R9B | NM_032595 | protein phosphatase | 495 | 496 |
| | | | 1, regulatory | | |
| | | | (inhibitor) subunit 9B | | |
| 150244 | FLJ31568 | NM_152509 | zinc finger, DHHC- | 497 | 498 |
| | | | type containing 8 | | |
| | | | pseudogene | | |
| 10137 | RBM12 | NM_006047 | RNA binding motif | 499 | 500 |
| | | NM_152838 | protein 12 | | |
| 83932 | DKFZP547N043 | NM_001010984 | Aka: chromosome 1 | 501 | 502 |
| | | NM_032018 | open reading frame | | |
| | | | 124 | | |
| 56135 | PCDHAC1 | NM_018898 | protocadherin alpha | 503 | 504 |
| | | NM_031882 | subfamily C, 1 | | |
| 10725 | NFAT5 | NM_001113178 | nuclear factor of | 505 | 506 |
| | | NM_006599 | activated T-cells 5, | | |
| | | NM_138713 | tonicity-responsive | | |
| | | NM_138714 | | | |
| | | NM_173214 | | | |
| 3273 | HRG | NM_000412 | histidine-rich | 507 | 508 |
| | | | glycoprotein | | |
| 65217 | PCDH15 | NM_001142763 | protocadherin 15 | 509 | 510 |
| | | NM_001142764. | | | |
| | | NM_001142765 | | | |
| | | NM_001142766 | | | |
| | | NM_001142767 | | | |
| | | NM_001142768 | | | |
| | | NM_001142769 | | | |
| | | NM_001142770 | | | |
| | | NM_001142771 | | | |
| | | NM_001142772 | | | |
| | | NM_001142773 | | | |
| | | NM_033056 | | | |
| 8336 | HIST1H2AM | NM_003514 | Histone cluster 1, | 511 | 512 |
| | | | H2am | | |
| 440515 | ZNF506 | NM_001099269 | Zinc finger protein | 513 | 514 |
| | | NM_001145404 | 506 | | |
| 23547 | ILT7 | NM_012276 | Aka: leukocyte | 515 | 516 |
| | | | immunoglobulin-like | | |
| | | | receptor, subfamily A | | |
| | | | (with TM domain), | | |
| | | | member 4 | | |
| 10436 | C2F | NM_006331 | Aka: EMG1 | 517 | 518 |
| | | | nucleolar protein | | |
| | | | homolog | | |
| 10571 | SMA3 | NM_006780 | SMA3/SMA4 | 519 | 520 |
| | | NM_021652 | glucuronidase | | |
| 688 | KLF5 | NM_001730 | Kruppel-like factor 5 | 521 | 522 |
| | | | (intestinal) | | |
| 373863 | DND1 | NM_194249 | Dead end homolog 1 | 523 | 524 |
| | | | (zebrafish) | | |
| 162979 | ZNF342 | NM_145288 | Aka: zinc finger | 525 | 526 |
| | | | protein 296 | | |
| 8826 | IQGAP1 | NM_003870 | IQ motif containing | 527 | 528 |
| | | | GTPase activating | | |
| | | | protein 1 | | |
| 27006 | FGF22 | NM_020637 | Fibroblast growth | 529 | 530 |
| | | | factor 22 | | |
| 4303 | MLLT7 | NM_005938 | Aka: forkhead box | 531 | 532 |
| | | | O4 | | |
| 9798 | KIAA0174 | NM_014761 | KIAA0174 | 533 | 534 |
| 388170 | LOC388170 | XM_373646 | hypothetical | 535 | 536 |
| | | | LOC388170 | | |
| 2956 | MSH6 | NM_000179 | MutS homolog 6 (E. coli) | 537 | 538 |
| 3897 | L1CAM | NM_000425 | L1 cell adhesion | 539 | 540 |
| | | NM_001143963 | molecule | | |
| | | NM_024003 | | | |
| 2160 | F11 | NM_000128 | Coagulation factor XI | 541 | 542 |
| 23435 | TARDBP | NM_007375 | TAR DNA binding | 543 | 544 |
| | | | protein | | |
| 3162 | HMOX1 | NM_002133 | Heme oxygenase | 545 | 546 |
| | | | (decycling) 1 | | |
| 8694 | DGAT1 | NM_012079 | Diacylglycerol O- | 547 | 548 |
| | | | acyltransferase | | |
| | | | homolog 1 (mouse) | | |
| 5289 | PIK3C3 | NM_002647 | Phosphoinositide-3- | 549 | 550 |
| | | | kinase, class 3 | | |
| 84460 | KIAA1789 | NM_001011657 | Aka: zinc finger, | 551 | 552 |
| | | NM_032441 | matrin type 1 | | |
| 3276 | HRMT1L2 | NM_001536 | Aka: protein arginine | 553 | 554 |
| | | NM_198318 | methyltransferase 1 | | |
| | | NM_198319 | | | |
| 7512 | XPNPEP2 | NM_003399 | X-prolyl | 555 | 556 |
| | | | aminopeptidase | | |
| | | | (aminopeptidase P) 2, | | |
| | | | membrane-bound | | |
| 3632 | INPP5A | NM_005539 | Inositol | 557 | 558 |
| | | | polyphosphate-5- | | |
| | | | phosphatase, 40 kDa | | |
| 10616 | C20ORF18 | NM_006462 | Aka: RanBP-type and | 559 | 560 |
| | | NM_031229 | C3HC4-type zinc | | |
| | | | finger containing 1 | | |
| 51366 | DD5 | NM_015902 | Aka: ubiquitin | 561 | 562 |
| | | | protein ligase E3 | | |
| | | | component n- | | |
| | | | recognin 5 | | |
| 4125 | MAN2B1 | NM_000528 | Mannosidase, alpha, | 563 | 564 |
| | | | class 2B, member 1 | | |
| 10633 | RRP22 | NM_001007279 | Aka: RAS-like, | 565 | 566 |
| | | NM_006477 | family 10, member A | | |
| 254225 | KIAA1991 | NM_001098638 | Aka: ring finger | 567 | 568 |
| | | | protein 169 | | |
| 4671 | BIRC1 | NM_004536 | Aka: NLR family, | 569 | 570 |
| | | NM_022892 | apoptosis inhibitory | | |
| | | | protein | | |
| 339318 | ZNF181 | NM_001029997 | Zinc finger protein | 571 | 572 |
| | | NM_001145665 | 181 | | |
| 135932 | FLJ90586 | NM_153345 | Aka: transmembrane | 573 | 574 |
| | | | protein 139 | | |
| 348180 | LOC348180 | NM_001012759 | chromosome 16 open | 575 | 576 |
| | | NM_001012762 | reading frame 84 | | |
| 2002 | ELK1 | NM_001114123 | ELK1, member of | 577 | 578 |
| | | NM_005229 | ETS oncogene family | | |
| 3034 | HAL | NM_002108 | Histidine ammonialyase | 579 | 580 |
| 11108 | PRDM4 | NM_012406 | PR domain | 581 | 582 |
| | | | containing 4 | | |
| 84639 | IL1F10 | NM_032556 | Interleukin 1 family, | 583 | 584 |
| | | NM_173161 | member 10 (theta) | | |
| 5087 | PBX1 | NM_002585 | Pre-B-cell leukemia | 585 | 586 |
| | | | homeobox 1 | | |
| 90317 | ZNF616 | NM_178523 | Zinc finger protein | 587 | 588 |
| | | | 616 | | |
| 10288 | LILRB2 | NM_001080978 | Leukocyte | 589 | 590 |
| | | NM_005874 | immunoglobulin-like | | |
| | | | receptor, subfamily B | | |
| | | | (with TM and ITIM | | |
| | | | domains), member 2 | | |
| 64582 | GPR135 | NM_022571 | G protein-coupled | 591 | 592 |
| | | | receptor 135 | | |
| 7082 | TJP1 | NM_003257 | tight junction protein | 593 | 594 |
| | | NM_175610 | 1 (zona occludens 1) | | |
| 400890 | LOC400890 | XM_379036 | LOC400890 | 595 | 596 |
| 6432 | SFRS7 | NM_001031684 | Splicing factor, | 597 | 598 |
| | | | arginine/serine-rich 7, | | |
| | | | 35 kDa | | |
| 8910 | SGCE | NM_001099400 | sarcoglycan, epsilon | 599 | 600 |
| | | NM_001099401 | | | |
| | | NM_003919 | | | |
| 408263 | MGC27121 | NM_001001343 | chromosome 5 open | 601 | 602 |
| | | | reading frame 40 | | |
| 83607 | MGC4268 | NM_031445 | AMME chromosomal | 603 | 604 |
| | | | region gene 1-like | | |
| 9825 | SPATA2 | NM_001135773 | spermatogenesis | 605 | 606 |
| | | NM_006038 | associated 2 | | |
| 5175 | PECAM1 | NM_000442 | platelet/endothelial | 607 | 608 |
| | | | cell adhesion | | |
| | | | molecule | | |
| 81575 | DKFZP434F0318 | NM_001130415 | apolipoprotein L | 609 | 610 |
| | | NM_030817 | domain containing 1 | | |
| 390033 | LOC390033 | XM_372345 | SSU72 RNA | 611 | 612 |
| | | | polymerase II CTD | | |
| | | | phosphatase homolog | | |
| 23607 | CD2AP | NM_012120 | CD2-associated | 613 | 614 |
| | | | protein | | |
| 401137 | LOC401137 | NM_214711 | chromosome 4 open | 615 | 616 |
| | | | reading frame 40 | | |
| 441734 | DKFZP434I1020 | XM_001715597 | LOC441734 similar | 617 | 618 |
| | | | to hypothetical | | |
| | | | protein | | |
| | | | DKFZp434I1020 | | |
| 7850 | IL1R2 | NM_004633 | interleukin 1 | 619 | 620 |
| | | | receptor, type II | | |
| 83593 | RASSF5 | NM_182663 | Ras association | 621 | 622 |
| | | NM_182664 | (RalGDS/AF-6) | | |
| | | NM_182665 | domain family | | |
| | | | member 5 | | |
| 387912 | LOC387912 | XM_370716 | hypothetical | 623 | 624 |
| | | | LOC387912 | | |
| 79095 | C9ORF16 | NM_024112 | chromosome 9 open | 625 | 626 |
| | | | reading frame 16 | | |
| 81698 | C15ORF5 | NM_030944 | chromosome 15 open | 627 | 628 |
| | | | reading frame 5 | | |
| 319101 | K6IRS3 | NM_175068 | Aka: keratin 73 | 629 | 630 |
| 3563 | IL3RA | NM_002183 | interleukin 3 | 631 | 632 |
| | | | receptor, alpha (low | | |
| | | | affinity) | | |
| 442191 | OR5U1 | NM_030946 | olfactory receptor, | 633 | 634 |
| | | | family 5, subfamily U | | |
| | | | member 1; replaced | | |
| | | | with olfactory | | |
| | | | receptor, family 14, | | |
| | | | subfamily J, member 1 | | |
| 89884 | LHX4 | NM_033343 | LIM homeobox 4 | 635 | 636 |
| 84189 | SLITRK6 | NM_032229 | SLIT and NTRK-like | 637 | 638 |
| | | | family, member 6 | | |
| 255626 | HIST1H2BA | NM_170610 | histone cluster 1, | 639 | 640 |
| | | | H2ba | | |
| 8139 | GAN | NM_022041 | gigaxonin | 641 | 642 |
| 399 | RHOH | NM_004310 | ras homolog gene | 643 | 644 |
| | | | family, member H | | |
| 200909 | HTR3D | NM_001145143 | 5-hydroxytryptamine | 645 | 646 |
| | | NM_182537 | (serotonin) receptor 3 | | |
| | | | family member D | | |
| 338785 | KRT6L | NM_175834 | Aka: keratin 79 | 647 | 648 |
| 284323 | LOC284323 | NM_001010880 | zinc finger protein | 649 | 650 |
| | | NM_001142577 | 780A | | |
| 669 | BPGM | NM_001724 | 2,3- | 651 | 652 |
| | | NM_199186 | bisphosphoglycerate | | |
| | | | mutase | | |
| 171568 | POLR3H | NM_001018050 | polymerase (RNA) | 653 | 654 |
| | | NM_001018052 | III (DNA directed) | | |
| | | NM_138338 | polypeptide H | | |
| | | | (22.9 kD) | | |
| 449520 | GGNBP1 | XM_001721178 | gametogenetin | 655 | 656 |
| | | XM_001721177 | binding protein 1 | | |
| 10180 | RBM6 | NM_005777 | RNA binding motif | 657 | 658 |
| | | | protein 6 | | |
| 4818 | NKG7 | NM_005601 | natural killer cell | 659 | 660 |
| | | | group 7 sequence | | |
| 27351 | D15WSU75E | NM_015704 | Aka: PPPDE | 661 | 662 |
| | | | peptidase domain | | |
| | | | conaining 2 | | |
| 26748 | GAGE7B | NM_001477 | Aka: G antigen 12I | 663 | 664 |
| 1388 | CREBL1 | NM_001136153 | Aka: activating | 665 | 666 |
| | | NM_004381 | transcription factor 6 | | |
| | | | beta | | |
| 1837 | DTNA | NM_001128175 | dystrobrevin, alpha | 667 | 668 |
| | | NM_001390 | | | |
| | | NM_001391 | | | |
| | | NM_001392 | | | |
| | | NM_032975 | | | |
| | | NM_032978 | | | |
| | | NM_032979 | | | |
| | | NM_032980 | | | |
| | | NM_032981 | | | |
| 1438 | CSF2RA | NM_006140 | colony stimulating | 669 | 670 |
| | | NM_172245 | factor 2 receptor, | | |
| | | NM_172246 | alpha, low-affinity | | |
| | | NM_172247 | (granulocyte- | | |
| | | NM_172249 | macrophage) | | |
| 23348 | DOCK9 | NM_001130048 | dedicator of | 671 | 672 |
| | | NM_001130049 | cytokinesis 9 | | |
| | | NM_001130050 | | | |
| | | NM_015296 | | | |
| 388337 | LOC388337 | XM_371018 | similar to CDRT15 | 673 | 674 |
| | | | protein | | |
| 10219 | KLRG1 | NM_005810 | killer cell lectin-like | 675 | 676 |
| | | | receptor subfamily G, | | |
| | | | member 1 | | |
| 26608 | WBSCR14 | NM_012453 | Aka: transducin | 677 | 678 |
| | | | (beta)-like 2 | | |
| 874 | CBR3 | NM_001236 | carbonyl reductase 3 | 679 | 680 |
| 10880 | ACTL7B | NM_006686 | actin-like 7B | 681 | 682 |
| 4609 | MYC | NM_002467 | v-myc | 683 | 684 |
| | | | myelocytomatosis | | |
| | | | viral oncogene | | |
| | | | homolog (avian) | | |
| 8936 | WASF1 | NM_001024934 | WAS protein family, | 685 | 686 |
| | | NM_001024935 | member 1 | | |
| | | NM_001024936 | | | |
| | | NM_003931 | | | |
| 4069 | LYZ | NM_000239 | lysozyme (renal | 687 | 688 |
| | | | amyloidosis) | | |
| 6448 | SGSH | NM_000199 | N-sulfoglucosamine | 689 | 690 |
| | | | sulfohydrolase | | |
| 7276 | TTR | NM_000371 | transthyretin | 691 | 692 |
| 30820 | KCNIP1 | NM_001034837 | Kv channel | 693 | 694 |
| | | NM_001034838 | interacting protein | | |
| | | NM_014592 | | | |
| 84264 | HAGHL | NM_032304 | hydroxyacylglutathione | 695 | 696 |
| | | NM_207112 | hydrolase-like | | |
| 3178 | HNRPA1 | NM_002136 | heterogeneous | 697 | 698 |
| | | NM_031157 | nuclear | | |
| | | | ribonucleoprotein A1 | | |
| 1462 | CSPG2 | NM_001126336 | versican | 699 | 700 |
| | | NM_004385 | | | |
| 7409 | VAV1 | NM_005428 | guanine nucleotide | 701 | 702 |
| | | | exchange factor | | |
| 10813 | UTP14A | NM_006649 | U3 small nucleolar | 703 | 704 |
| | | | ribonucleoprotein, | | |
| | | | homolog A (yeas | | |
| 388235 | LOC388235 | XM_373672 | hypothetical | 705 | 706 |
| | | | LOC643015 | | |
| 80833 | APOL3 | NM_014349 | apolipoprotein L, 3 | 707 | 708 |
| | | NM_030644 | | | |
| | | NM_145639 | | | |
| | | NM_145640 | | | |
| | | NM_145641 | | | |
| | | NM_145642 | | | |
| 144132 | FLJ32752 | NM_144666 | Aka: dynein heavy | 709 | 710 |
| | | NM_173589 | chain domain 1 | | |
| 5524 | PPP2R4 | NM_021131 | protein phosphatase | 711 | 712 |
| | | NM_178000 | 2A activator, | | |
| | | NM_178001 | regulatory subunit | | |
| | | NM_178003 | | | |
| 5024 | P2RX3 | NM_002559 | purinergic receptor | 713 | 714 |
| | | | P2X, ligand-gated ion | | |
| | | | channel | | |
| 79844 | ZDHHC11 | NM_024786 | zinc finger, DHHC- | 715 | 716 |
| | | | type containing 11 | | |
| 4858 | NOVA2 | NM_002516 | NOVA2 neuro- | 717 | 718 |
| | | | oncological ventral | | |
| | | | antigen | | |
| 8727 | CTNNAL1 | NM_003798 | Catenin (cadherin- | 719 | 720 |
| | | | associated protein), | | |
| | | | alpha-like 1 | | |
| 4254 | KITLG | NM_000899 | Kit ligand | 721 | 722 |
| | | NM_003994 | | | |
| 55781 | RIOK2 | NM_018343 | RIO kinase 2 | 723 | 724 |
| 79792 | GSDMDC1 | NM_024736 | Gasdermin D | 725 | 726 |
| 23649 | POLA2 | NM_002689 | Polymerase (DNA | 727 | 728 |
| | | | directed), alpha 2 | | |
| | | | (70 kD subunit) | | |
| 9948 | WDR1 | NM_005112 | WD repeat domain 1 | 729 | 730 |
| | | NM_017491 | | | |
| 29940 | SART2 | NM_001080976 | Dermatan sulfate | 731 | 732 |
| | | NM_013352 | epimerase | | |
| 6900 | CNTN2 | NM_005076 | Contactin 2 (axonal) | 733 | 734 |
| 886 | CCKAR | NM_000730 | Cholecystokinin A | 735 | 736 |
| | | | receptor | | |
| 87 | ACTN1 | NM_001102 | Actinin, alpha 1 | 737 | 738 |
| | | NM_001130004 | | | |
| | | NM_001130005 | | | |
| 2266 | FGG | NM_000509 | Fibrinogen gamma | 739 | 740 |
| | | NM_021870 | chain | | |
| 2936 | GSR | NM_000637 | Glutathione reductase | 741 | 742 |
| 1489 | CTF1 | NM_001142544 | Cardiotrophin 1 | 743 | 744 |
| | | NM_001330 | | | |
| 29982 | NRBF2 | NM_030759 | Nuclear receptor | 745 | 746 |
| | | | binding factor 2 | | |
| 3479 | IGF1 | NM_000618 | Insulin-like growth | 747 | 748 |
| | | NM_001111283 | factor 1 | | |
| | | NM_001111284 | (somatomedin C) | | |
| | | NM_001111285 | | | |
| 65082 | VPS33A | NM_022916 | Vacuolar protein | 749 | 750 |
| | | | sorting 33 homolog A | | |
| 51465 | UBE2J1 | NM_016021 | Ubiquitin- | 751 | 752 |
| | | | conjugating enzyme | | |
| | | | E2, J1 | | |
| 10205 | EVA1 | NM_005797 | Myelin protein zero- | 753 | 754 |
| | | NM_144765 | like 2 | | |
| 6363 | CCL19 | NM_006274 | Chemokine (C-C | 755 | 756 |
| | | | motif) ligand 19 | | |
| 5095 | PCCA | NM_000282 | Propionyl Coenzyme | 757 | 758 |
| | | NM_001127692 | A carboxylase, alpha | | |
| | | | polypeptide | | |
| 150681 | OR6B3 | NM_173351 | Olfactory receptor, | 759 | 760 |
| | | | family 6, subfamily | | |
| | | | B, member 3 | | |
| 29075 | HSPC072 | NM_014162 | LOC29075 | 761 | 762 |
| 57475 | PLEKHH1 | NM_020715 | Pleckstrin homology | 763 | 764 |
| | | | domain containing, | | |
| | | | family H (with | | |
| | | | MyTH4 domain) | | |
| | | | member 1 | | |
| 1183 | CLCN4 | NM_001830 | Chloride channel 4 | 765 | 766 |
| 341152 | OR2AT4 | NM_001005285 | Olfactory receptor, | 767 | 768 |
| | | | family 2, subfamily | | |
| | | | AT, member 4 | | |
| 84226 | C2ORF16 | NM_032266 | Chromosome 2 open | 769 | 770 |
| | | | reading frame 16 | | |
| 400550 | LOC400550 | XM_001128652 | FLJ34515 | 771 | 772 |
| | | | hypothetical gene | | |
| | | | supported by | | |
| | | | AK091834 | | |
| 2905 | GRIN2C | NM_000835 | Glutamate receptor, | 773 | 774 |
| | | | ionotropic, N-methyl | | |
| | | | D-aspartate 2C | | |
| 79841 | FLJ23598 | NM_024783 | ATP/GTP binding | 775 | 776 |
| | | | protein-like 2 | | |
| 7248 | TSC | NM_000368 | Tuberous sclerosis 1 | 777 | 778 |
| | | NM_001008567 | | | |
| 2652 | OPN1MW | NM_000513 | Opsin 1 (cone | 779 | 780 |
| | | | pigments), medium- | | |
| | | | wave-sensitive | | |
| 9955 | HS3ST3A1 | NM_006042 | Heparan sulfate | 781 | 782 |
| | | | (glucosamine) 3-O- | | |
| | | | sulfotransferase 3A1 | | |
| 10395 | DLC1 | NM_006094 | Deleted in liver | 783 | 784 |
| | | NM_024767 | cancer 1 | | |
| | | NM_182643 | | | |
| 10360 | NPM3 | NM_006993 | Nucleophosmin/nucle | 785 | 786 |
| | | | oplasmin, 3 | | |
| 3630 | INS | NM_000207 | Insulin | 787 | 788 |
| 54471 | FLJ20232 | NM_019008 | Smith-Magenis | 789 | 790 |
| | | | syndrome | | |
| | | | chromosome region, | | |
| | | | candidate 7-like | | |
| 24 | ABCA4 | NM_000350 | ATP-binding | 791 | 792 |
| | | | cassette, sub-family | | |
| | | | A (ABC1), member 4 | | |
| 6713 | SQLE | NM_003129 | Squalene epoxidase | 793 | 794 |
| 57494 | KIAA1238 | NM_020734 | Ribosomal | 795 | 796 |
| | | | modification protein | | |
| | | | rimK-like family | | |
| | | | member B | | |
| 81850 | KRTAP1-3 | NM_030966 | Keratin associated | 797 | 798 |
| | | | protein 1-3 | | |
| 23151 | KIAA0767 | NM_015124 | GRAM domain | 799 | 800 |
| | | | containing 4 | | |
| 2689 | GH2 | NM_002059 | Growth hormone 2 | 801 | 802 |
| | | NM_022556 | | | |
| | | NM_022557 | | | |
| | | NM_022558 | | | |
| 100101629 | GAGE8 | NM_012196 | G antigen 8 | 803 | 804 |
| 140691 | LOC400368 | NM_080745 | TRIM69 tripartite | 805 | 806 |
| | | NM_182985 | motif-containing 69 | | |
| 56300 | IL1F9 | NM_019618 | Interleukin 1 family, | 807 | 808 |
| | | | member 9 | | |
| 55777 | MBD5 | NM_018328 | Methyl-CpG binding | 809 | 810 |
| | | | domain protein 5 | | |
| 10380 | BPNT1 | NM_006085 | 3′(2′), 5′-bisphosphate | 811 | 812 |
| | | | nucleotidase 1 | | |
| 167127 | LOC167127 | NM_174914 | UGT3A2 UDP | 813 | 814 |
| | | | glycosyltransferase 3 | | |
| | | | family, polypeptide | | |
| | | | A2 | | |
| 124220 | LOC124220 | NM_145252 | zymogen granule | 815 | 816 |
| | | | protein 16 homolog B | | |
| | | | (rat) | | |
| 11335 | CBX3 | NM_007276 | Chromobox homolog | 817 | 818 |
| | | NM_016587 | 3 (HP1 gamma | | |
| | | | homolog) | | |
| 55752 | SEPT11 | NM_018243 | Septin 11 | 819 | 820 |
| 1277 | COL1A1 | NM_000088 | Collagen, type I, | 821 | 822 |
| | | | alpha 1 | | |
| 2566 | GABRG2 | NM_000816 | gamma-aminobutyric | 823 | 824 |
| | | NM_198903 | acid (GABA) A | | |
| | | NM_198904 | receptor, gamma 2 | | |
| 338339 | CLECSF8 | NM_080387 | Aka: C-type lectin | 825 | 826 |
| | | | domain family 4, | | |
| | | | member | | |
| 54829 | ASPN | NM_017680 | asporin | 827 | 828 |
| 50615 | IL21R | NM_021798 | interleukin 21 | 829 | 830 |
| | | NM_181078 | receptor | | |
| | | NM_181079 | | | |
| 57495 | KIAA1239 | NM_001144990 | KIAA1239 | 831 | 832 |
| 84885 | ZDHHC12 | NM_032799 | zinc finger, DHHC- | 833 | 834 |
| | | | type containing 12 | | |
| 150135 | C21ORF129 | NR_027272 | C21orf129 | 835 | |
| | | | chromosome 21 open | | |
| | | | reading frame 129 | | |
| 158228 | C9ORF122 | NR_027294 | C9orf122 | 836 | |
| | | | chromosome 9 open | | |
| | | | reading frame 122 | | |
| 347918 | FLJ33915 | NR_003290 | EP400NL EP400 N- | 837 | |
| | | | terminal like | | |
| 11039 | SMA4 | | Glucuronidase, beta | 838 | |
| | | | pseudogene | | |
| 400590 | LOC400590 | XR_042032 | hypothetical | 839 | |
| | | XR_042034 | LOC400590 | | |
| | | XR_042033 | | | |
| 643015 | LOC389900 | XR_016273 | hypothetical | 840 | |
| | | XR_018290 | LOC643015 | | |
| | | XR_037152 | | | |
| 284912 | LOC284912 | XR_1041187 | hypothetical | 841 | |
| | | | LOC284912 | | |
| 400433 | LOC400433 | XM_378538 | DNM1P40 DNM1 | 842 | |
| | | | pseudogene 40 | | |

The initial screen used two independent siRNAs for each target gene and the assays were performed in duplicate. Subsequent follow up validation assays for positive hits were performed with a total of four siRNAs against each target in triplicate (discussed in Example 4). HDAC1 siRNA served a positive control and GAPDH siRNA served as a negative control.

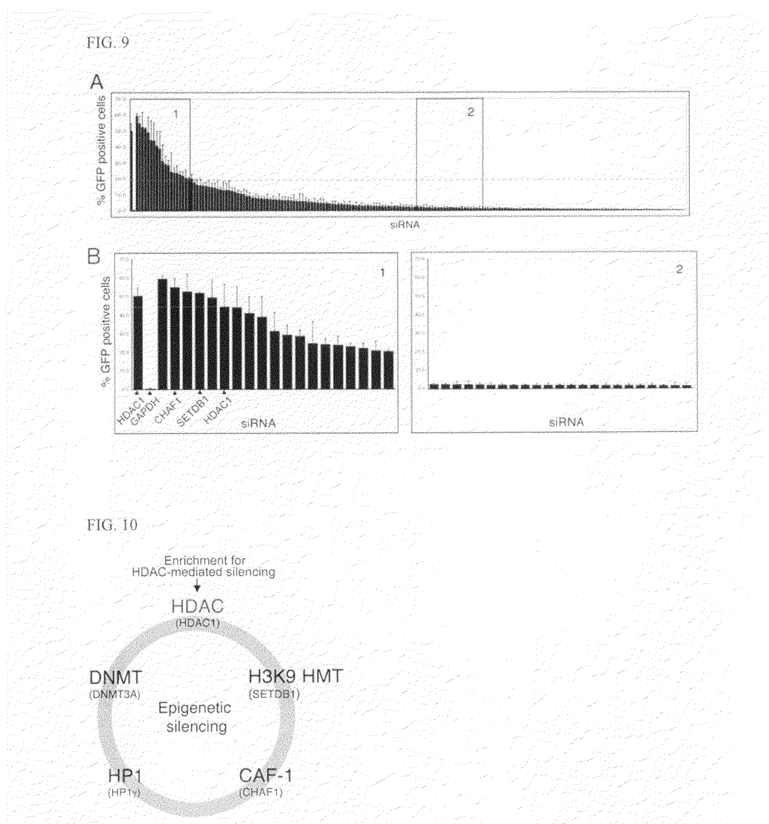

FIG. 9A depicts raw data from one of the two independent siRNAs series comprising the 189 pre-selected epigenetics siRNA set. The screen was carried out in duplicate (error bars are shown) and the results are ranked based on the percent GFP positive cells (scoring reactivation from the silent state). The results with the second siRNA set (denoted “replicates”) are not shown for simplicity, but detailed analysis with up to four independent siRNAs are shown with the next example. In Panels A and B, Group 1 defines siRNAs that produced >20% GFP reactivation. The siRNAs in this group, along those identified in the replicate siRNA set, were considered for further analyses.

Overall, 16 gene hits (16/189) were identified as defined by a three criteria: i) the semi-arbitrary cutoff of GFP reactivation in at least 20% of the cells; ii) reproducible reactivation with at least two independent siRNAs per target; and iii) the mean fluorescence intensity (MFI). In addition to validation tests with a total of 4 siRNAs/target, the two secondary assays were employed to detect false negatives as described above. Unless specified, all hits in the screen could be reproduced with two independent cell populations in which the silent GFP was under control of different promoters. The hits are discussed in detail in Example 4, but first the general profile of the assay, validation and secondary assays is discussed.

As shown in FIG. 9, screening of this pre-selected epigenetic siRNA set did not produce an all-or-none binary readout, but rather, a graded reactivation response was noted among the hits. This profile is expected, as target proteins have different half lives of decay after mRNA knockdown and only one time point was selected for GFP readout (96 hours). Furthermore the diverse biological roles of the targets in silencing may not produce identical effects in the assay. For example, some epigenetic regulators could have cell-cycle specific roles, and as such only a subset of cells may be sensitive within the course of the experiment. Overall, the screen provides a broad snapshot, and is not universally optimized for measuring the effect of each siRNA.

Another parameter of the screen that was considered is the expression profile of target genes in the HeLa reporter cells. For example, a non-hit could reflect the fact that the gene is simply not expressed. For the proof of concept studies (Example 1), it was confirmed that the expression of targets using extensive qRT-PCR and western analysis, both pre- and post-siRNA knockdown. However, to avoid labor intensive and expensive screening of the entire library for evidence of expression of target genes, published or publicly available resources have thus far been used to assess this parameter. For example, the available microarray data and compiled evidence, for protein expression from published or commercial sources was used. As an example, in the proof of concept studies, it was confirmed that HDAC1, 2, 3, and 4 are expressed in HeLa cells (see Example 1). Public microarray data relevant to the remaining HDAC family members (HDAC 5, 6, 7, 8, 9, 10, 11) was not in agreement. However, evidence was found for protein expression of all HDACs in HeLa cells by surveying commercial sources for HDAC antibodies.

The proof-of-concept experiments indeed confirmed the involvement of HDAC1 (see Example 1). However, it seemed possible that additional hits might be limited to factors that that are present in HDAC co-repressor complexes. This was not the case. Instead, the screen has identified additional codependent factors, possibly reflecting what has been described as a “cooperative and self-reinforcing organization of the chromatin and DNA modifying machinery,” as described below.

### Example 4

This example describes the validation of several modulators of epigenetic silencing identified as described in Example 3. Below, eight of the 16 validated hits from the epigenetics siRNA set are discussed.

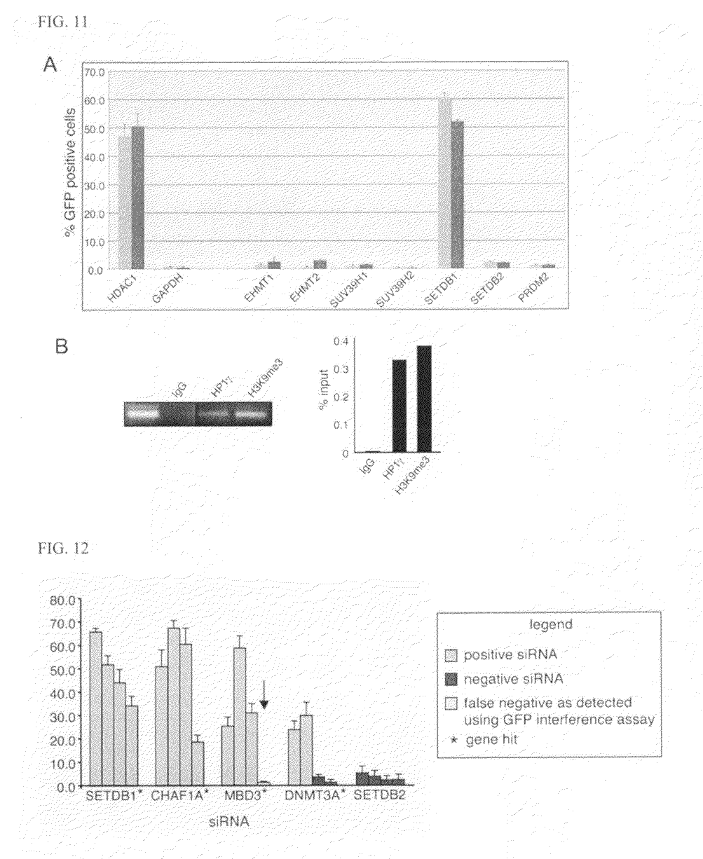

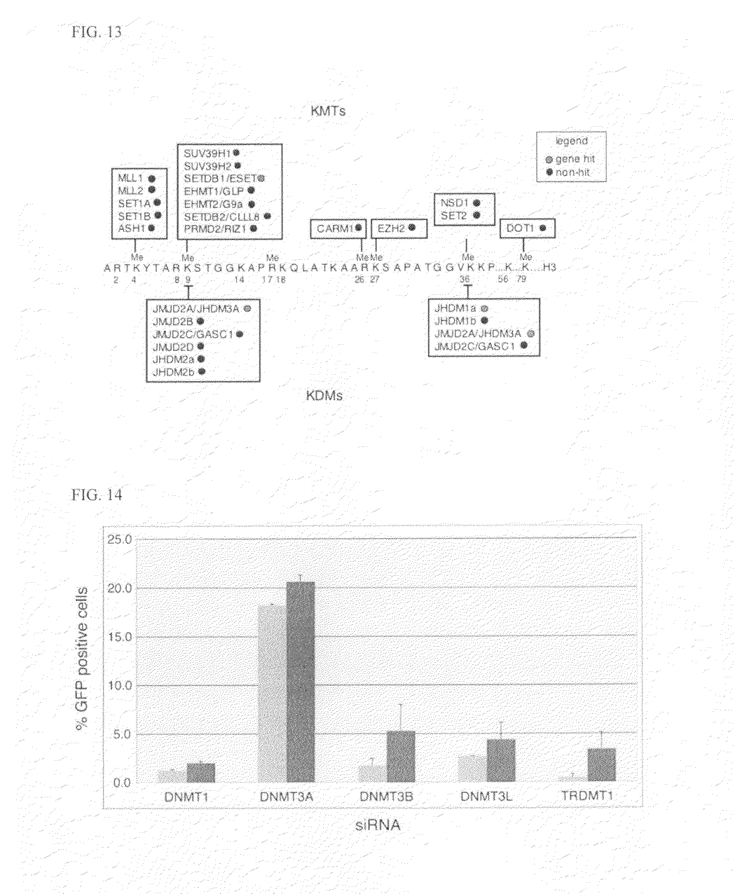

Among the 16 validated hits was SETDB1, a histone methyltransferase (HMT) that mediates H3K9 methylation (FIG. 9, 11A). This finding indicates a role for the repressive H3K9 methylation in silencing of the GFP reporter gene. This gene hit was confirmed with four independent siRNAs (FIG. 12). None of the other known H3K9 HMT family members scored in the assay (FIG. 11A). As a further comparison, it is shown in diagrammatic fashion, all of the siRNAs that were included in the pre-selected siRNA set that target enzymes involved in methylation and demethylation of the H3 N-terminal tail (FIG. 12). As H3K9 is generally associated with transcriptional repression, it was anticipated that potential hits would include H3K9 HMTs, but not H3K9 demethylases, and that is indeed what was observed (FIG. 13). Similarly, as H3K4 methylation is associated with the start sites of active, or “primed” genes. As such, H3K4 HMTs would not be expected to play a role in silencing. The results in FIGS. 11A and 13 thereby highlight the high degree of specificity and functional relevance revealed by this siRNA-based screen. A provisional hit with JHDM1b siRNA (FIG. 13) was also observed. This enzyme is a HDM that acts on H3K36 methyl substrates. The H3K36 methyl modification is associated with the body of active genes and it is therefore possible that the H3K36 demethylase could play a repressive role.

As mentioned in Example 1, a role for HP1γ in silencing (using siRNA and other methods) was identified, supporting a generally accepted model whereby the H3K9 mark provides a binding site for HP1. In this case, the SETDB1 would serve as the “writer,” with HP1 serving as an “effector.” HP1 could drive formation of repressive chromatin or recruit other repressive factors. To evaluate further the significance of these hits, chromatin immunoprecipitation (ChIP) assays were performed to measure HP1 occupancy at the silent GFP promoter. Preliminary results shown in FIG. 11B indicate that the H3K9 trimethyl mark and HP1γ are present at the promoter of the silent GFP gene.

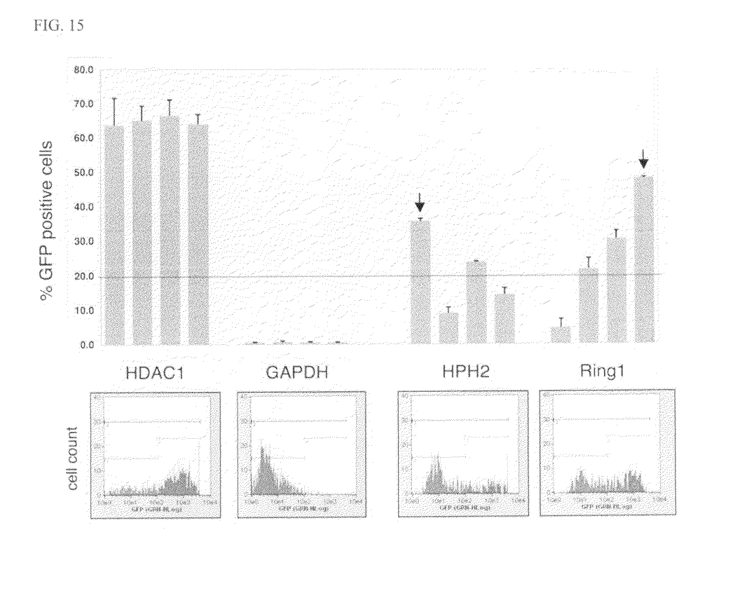

Among the other hits was a DNA methyltransferase, DNMT3A (FIG. 7B). This DNMT has been considered a de novo DNMT rather than a maintenance methylase; however DNMT3A has been implicated in silencing and can be localized to certain silent loci. As mentioned above, the intensity of the GFP response varied, as measured by percentage of cells in which GFP is reactivated. The DNMT3A siRNAs produced a characteristic weaker reactivation (FIG. 12). It is possible that the lower level of reactivation may reflect a requirement for significant dilution of existing DNA methylation pattern via multiple S-phases, subsequent to DNMT knockdown. An alternative explanation for the limited response to DNMT3A siRNA is that this protein plays a role in only a subset of cells in the population. To further evaluate the significance of this hit, the effects of knockdown of other DNMT family members (FIG. 14) was compared. As shown, the two independent DNMT3A siRNA replicates produced a similar level of reactivation (in duplicate tests), while no significant effects were observed with siRNAs targeting other family members.

DNMTs can play both enzymatic and non-enzymatic roles in epigenetic silencing. Non-enzymatic functions include recruitment of repressive factors, such as HDACs. As siRNA knockdown of DNMT3A would affect both enzymatic and non-enzymatic activities of DNMT3A, DNMT inhibitors were used to investigate an enzymatic role. Optimization of inhibitor concentrations and prolonged treatment revealed a similar level of reactivation as was observed with DNMT3A siRNA. This is indicative that DNMT3A enzymatic activity may contribute to silencing in this system. As was the case with HDAC siRNAs and HDAC inhibitors, the DNMT siRNA phenocopies the effect of a DNMT inhibitor. Such parallel chemical and siRNA approaches are generally used to identify drug targets and here have been used for the same principle to reinforce the preliminary interpretations regarding the roles of silencing factors.

A validated hit was also detected for another silencing factor, a DNA methyl binding domain (MBD) protein (FIG. 12), which may play several roles in silencing, including as an adapter for recognition of methylated DNA. In this case, the secondary assay for interference with GFP readout indicated that one of the four MBD siRNAs produced a false negative hit (FIG. 12). Thus, this hit was confirmed with a minimum of three independent siRNAs, with the fourth siRNA being uninformative.

Another strong and validated hit was CHAF1A (FIGS. 9 and 12). This gene encodes the CAF-1 p150 subunit of the CAF-1 histone chaperone. Previously, the p150 subunit was implicated in transfer of the SETDB1-mediated H3K9 methylation during S-phase. Thus, the screen has revealed a role for a non-enzymatic factor whose role is to participate in transfer or “inheritance” of marks. Furthermore CAF-1 p150 is known to functionally interact with another gene hit, SETDBI.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of identifying a gene product that is involved in epigenetic silencing, comprising:
providing a cell line comprising a genome into which is integrated an epigenetically silent reporter gene;
providing a mRNA inhibitor capable of inhibiting expression of a target mRNA, the product of which is suspected of being involved in the epigenetic silencing;
introducing the mRNA inhibitor into the cell line, thereby inhibiting expression of the target RNA; and
detecting an increase in expression of the reporter gene, the increase in expression being indicative that the product of expression of the target mRNA is involved in the epigenetic silencing.

2. The method of claim 1, wherein the cell line is of human origin.

3. The method of claim 1, wherein the cell line is a HeLa cell line.

4. The method of claim 1, wherein the silent reporter gene encodes a green fluorescent protein.

5. The method of claim 1, wherein the silent reporter gene is disposed within a retroviral vector.

6. The method of claim 1, wherein the silent reporter gene is operably linked to a promoter selected from a viral LTR promoter, a hCMV promoter, a EF1α promoter and a RNA Pol II promoter.

7. The method of claim 1, wherein the mRNA inhibitor is an antisense molecule, an siRNA, a miRNA or a ribozyme.

8. The method of claim 1, wherein the target mRNA comprises one or more of a mRNA encoding HDAC1, daxx or HP1γ.

9. The method of claim 1, wherein the target mRNA comprises one or more of a mRNA exemplified by SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 836, 837, 838, 839, 840, 841 and 842.

10. The method of claim 7, wherein the mRNA inhibitor comprises two or more siRNA targeting the same target mRNA.

11. The method of claim 1, adapted to comprise a high-throughput screening system, comprising a plurality of assay chambers in which each assay chamber comprises cells of the cell line into which different mRNA inhibitors are introduced.

12. A kit for identifying a gene product that is involved in epigenetic silencing, comprising a container and instructions, and further comprising one or more of (1) a cell line comprising a genome into which is integrated an epigenetically silent reporter gene; and (2) a mRNA inhibitor capable of inhibiting expression of a target mRNA, the product of which is suspected of being involved in the epigenetic silencing.

13. The kit of claim 12, adapted for practicing the method of claim 1 in plurality, comprising a plurality of assay containers and a plurality of mRNA inhibitors.

14. The kit of claim 13, comprising a multi-well plate, wherein the reporter gene encodes a gene product that is directly or indirectly fluorescently detectable.

15. A gene product that functions in maintaining epigenetic silencing, selected from HDAC1, Daxx, HP1γ, MBD1, MBD2, MBD3, SETDB1 (also known as ESET or KMT1E), DNMT3A, RING1, PHC2 (also known as HPH2), CHAF1A (also known CAF-1 p150), TRIM24 (also known as TIF1alpha), TRIM33 (also known as TIF1gamma), JMJD2A (also known as KDM4A), SUV420H2 (also known as KMT5C), RAD21, FBXL11 (also known as JHDM1a and KDM2A), PBRM1 (also known as BAF180) and ZMYND8.

16. The gene product of claim 15, selected from MBD1, MBD2, MBD3, SETDB1 (also known as ESET or KMT1E) and DNMT3A.

17. The gene product of claim 15, selected from RING1, PHC2 (also known as HPH2) and CHAF1A (also known CAF-1 p150).

18. The gene product of claim 15, selected from TRIM24 (also known as TIF1alpha), TRIM33 (also known as TIF1gamma), JMJD2A (also known as KDM4A) and SUV420H2 (also known as KMT5C).

19. The gene product of claim 15, selected from RAD21 and FBXL11 (also known as JHDM1a and KDM2A).

20. The gene product of claim 15, selected from PBRM1 (also known as BAF180) and ZMYND8.

21. A method of relieving epigenetic silencing in a cell, the method comprising contacting a cell with at least one nucleic acid inhibitor, wherein the nucleic acid inhibitor inhibits production or expression of one or more mRNA molecule in the cell, wherein the mRNA molecule in the cell is selected from the group consisting of SEQ NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 836, 837, 838, 839, 840, 841 and 842, and wherein the inhibition results in relief of epigenetic silencing in the cell.

22. The method of claim 21, wherein the cell is a human cell.

23. The method of claim 21, wherein the nucleic acid inhibitor is at least one of the group consisting of an antisense molecule, an siRNA, a miRNA or a ribozyme.

24. A method of relieving epigenetic silencing in a cell, the method comprising contacting a cell with at least one nucleic acid inhibitor, wherein the nucleic acid inhibitor inhibits production or expression of one or more mRNA molecule in the cell, wherein the mRNA molecule in the cell is selected from the group consisting of HDAC1, Daxx, HP1γ, MBD1, MBD2, MBD3, SETDB1 (also known as ESET or KMT1E), DNMT3A, RING1, PHC2 (also known as HPH2), CHAF1A (also known CAF-1 p150), TRIM24 (also known as TIF1alpha), TRIM33 (also known as TIF1 gamma), JMJD2A (also known as KDM4A), SUV420H2 (also known as KMT5C), RAD21, FBXL11 (also known as JHDM1a and KDM2A), PBRM1 (also known as BAF180) and ZMYND8, and wherein the inhibition results in relief of epigenetic silencing in the cell.

25. The method of claim 24, wherein the cell is a human cell.

26. The method of claim 24, wherein the nucleic acid inhibitor is at least one of the group consisting of an antisense molecule, an siRNA, a miRNA or a ribozyme.
